(19) United States
(12) Patent Application Publication
(10) Pub. No.: US 2006/0052314 A1
(43) Pub. Date: Mar. 9, 2006

(54) Pharmaceutical combination

(76) Inventors: Ann-Charlotte Roth-Rosendahl, Molndal (SE); Elisabeth Svernhage, Molndal (SE)

Correspondence Address: FISH & NEAVE IP GROUP;ROPES & GRAY LLP, ONE INTERNATIONAL PLACE, BOSTON, MA, 02110-2624, US

(21) Appl. No.: 10/516,426

(22) PCT Filed: May 27, 2003
(86) PCT No.: PCT/SE03/00854

§ 371 Date: Jun. 28, 2005

(30) Foreign Application Priority Data
May 31, 2002 (SE) 0201662-4

(51) Int. Cl. A61K 38/04; A61K 31/397

(52) U.S. Cl. 514/19; 514/210.17; 514/210.18

(57) ABSTRACT

There is provided a combination product comprising: (1) a compound of claim 1 in WO 02/44145 or a compound of claim 20 in WO 02/44145 (or derivateive thereof) or a pharmaceutically-acceptable derivative thereof; and (1) a compound as defined in claim 1 or WO 01/28993 or (2) compound of claim 34 or WO 01/28992 or (3) Compound A or B or C or D (or pharmaceutically-acceptable salts therof) for use in treating arrhythmia or a coagulation controlled complicaiton thereof.

### FIELD OF THE INVENTION

This invention relates to a new combination of pharmaceutically-active compounds. In particular the invention relates to a combination of thrombin inhibitor of a particular class or a pharmaceutically-acceptable derivative thereof and certain antiarrhythmic oxabispidines or pharmaceutically acceptable salts thereof.

### BACKGROUND OF THE INVENTION

Atrial fibrillation (AF) is characterised by grossly disorganised atrial electrical activity that is irregular in respect of both rate and rhythm. Patients with AF have no visually discernible timing pattern in atrial electrical activity when measured by surface ECG, or in electrogram sequences recorded by catheter electrodes.

During AF, the regular pumping action of the atria is replaced by irregular, disorganised and quivering spasms of atrial tissue. These spasms may be experienced as irregular heartbeat, palpitations, discomfort, dizziness and/or angina pectoris. Further, the inefficient pumping action of the heart tends to lead to significant morbidity related to reduced blood flow. More seriously, the reduced cardiac output can lead to blood pooling in the left atria and the formation of blood clots. Blood clots, mostly originating in the left atrium, can dislodge and travel through the bloodstream to organs, e.g. the brain, spleen, kidneys etc. If the clot travels to the brain, this may result in cerebral stroke and even death.

In the US alone, AF affects an estimated two million people, with approximately 160,000 new cases being diagnosed each year. It has been estimated that AF is responsible for over 70,000 strokes each year in the US, and that the cost of treating these patients is more than US$3.6 billion annually. The cost of drug treatment for AF alone has been estimated to be in excess of US$400 million world-wide each year.

AF can be classified in two broadly defined groups: “valvular” AF and “non-valvular” AF (NVAF). In valvular AF, the arrhythmia is experienced due to a disorder of one or more of the heart valves (e.g. valvular disease), or the presence of mechanical (prosthetic) heart valves. Conversely, NVAF is AF experienced in the case where there is an absence of significant valvular disease or prosthesis.

The oxabispidine compounds of international patent application WO 01/28992 are indicated as being useful in the treatment of cardiac arrhythmias. WO 01/28992 is incorporated herein by reference. Claim 1 of WO 01/28992 reads: A compound of formula I, 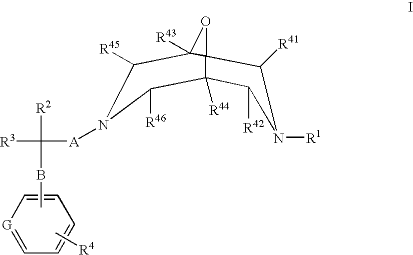
wherein

R¹ represents C₁₋₁₂ alkyl (which alkyl group is optionally substituted and/or terminated by one or more groups selected from halo, cyano, nitro, aryl, Het¹, —C(O)R^{5a}, —OR^{5b}, —N(R⁶)R^{5c}, —C(O)XR⁷, —C(O)N(R⁸)R^{5d}, and

—S(O)₂R⁹), or R¹ represents —C(O)XR⁷, —C(O)N(R⁸)R^{5d} or —S(O)₂R⁹;

R^{5a} to R^{5d} independently represent, at each occurrence, H, C₁₋₆ alkyl (which latter group is optionally substituted and/or terminated by one or more substituents selected from —OH, halo, cyano, nitro, aryl and Het²), aryl or Het³, or R^{5d}, together with R⁸, represents C₃₋₆ alkylene (which alkylene group is optionally interrupted by an O atom and/or is optionally substituted by one or more C₁₋₃ alkyl groups);

R⁶ represents H, C₁₋₆ alkyl (optionally substituted and/or terminated by one or more substituents selected from —OH, halo, cyano, nitro and aryl), aryl, —C(O)R^{10a}, —C(O)OR^{10b} or —C(O)N(H)R^{10e};

R^{10a}, R^{10b} and R^{10c} independently represent C₁₋₆ alkyl (optionally substituted and/or terminated by one or more substituents selected from —OH, halo, cyano, nitro and aryl), aryl, or R^{10a} represents H;

R⁷ represents C₁₋₁₂ alkyl (optionally substituted and/or terminated by one or more substituents selected from —OH, halo, cyano, nitro, aryl, C₁₋₆ alkoxy and Het⁴);

R⁸ represents H, C₁₋₁₂ alkyl, C₁₋₆ alkoxy (which latter two groups are optionally substituted and/or terminated by one or more substituents selected from —OH, halo, cyano, nitro, C₁₋₄ alkyl and C₁₋₄ alkoxy),

-D-aryl, -D-aryloxy, -D-Het⁵, -D-N(H)C(O)R^{11a}, -D-S(O)₂R^{12a},

-D-C(O)R^{11b}, -D-C(O)OR^{12b}, -D-C(O)N(R^{11c})R^{11d}, R⁸, together with R^{5d}, represents C₃₋₆ alkylene (which alkylene group is optionally interrupted by an O atom and/or is optionally substituted by one or more C₁₋₃ alkyl groups);

R^{11a} to R^{11d} independently represent H, C₁₋₆ alkyl (optionally substituted and/or terminated by one or more substituents selected from —OH, halo, cyano, nitro and aryl), aryl, or R^{11c} and R^{11d} together represent

C₃₋₆ alkylene;

R⁹, R^{12a} and R^{12b} independently represent C₁₋₆ alkyl (optionally substituted and/or terminated by one or more substituents selected from —OH, halo, cyano, nitro and aryl) or aryl;

D represents a direct bond or C₁₋₆ alkylene;

X represents O or S;

R² represents H, halo, C₁₋₆ alkyl, —OR¹³, -E-N(R¹⁴)R¹⁵ or, together with R³, represents ═O;

R³ represents H, C₁₋₆ alkyl or, together with R², represents ═O;

R¹³ represents H, C₁₋₆ alkyl, -E-aryl, -E-Het⁶, —C(O)R^{16a}, —C(O)OR^{16b} or —C(O)N(R^{17a})R^{17b};

R¹⁴ represents H, C₁₋₆ alkyl, -E-aryl, -E-Het⁶, —C(O)R^{16a}, —C(O)OR^{16b}, —S(O)₂R^{16c}, —[C(O)]ₚN(R^{17a})R^{17b} or —C(NH)NH₂;

R¹⁵ represents H, C₁₋₆ alkyl, -E-aryl or —C(O)R^{16d};

R¹⁶a to R^{16d} independently represent, at each occurrence when used herein, C₁₋₆ alkyl (optionally substituted and/or terminated by one or more substituents selected from halo, aryl and Het⁷), aryl, Het⁸, or R^{16a} and R^{16d} independently represent H;

R^{17a} and R^{17b} independently represent, at each occurrence when used herein, H or C₁₋₆ alkyl (optionally substituted and/or terminated by one or more substituents selected from halo, aryl and Het⁹), aryl, Het¹⁰, or together represent C₃₋₆ alkylene, optionally interrupted by an O atom;

E represents, at each occurrence when used herein, a direct bond or C₁₋₄ alkylene;

p represents 1 or 2;

Het¹ to Het¹⁰ independently represent five- to twelve-membered heterocyclic groups containing one or more heteroatoms selected from oxygen, nitrogen and/or sulfur, which groups are optionally substituted by one or more substituents selected from —OH, oxo, halo, cyano, nitro,

C₁₋₆ alkyl, C₁₋₆ alkoxy, aryl, aryloxy, —N(R^{18a})R^{18b}, —C(O)R^{18c}, —C(O)OR^{18d}, C(O)N(R^{18e})R^{18f}, —N(R^{18g})C(O)R^{18h} and —N(R¹⁸ⁱ)S(O)₂R^{18j};

R^{18a} to R^{18j} independently represent C₁₋₆ alkyl, aryl or R^{18a} to R¹⁸ⁱ independently represent H;

A represents a direct bond, -J-, -J-N(R¹⁹)- or -J-O— (in which latter two groups, N(R¹⁹)— or O— is attached to the carbon atom bearing R² and R³);

B represents -Z-, -Z-N(R²⁰)—, —N(R²⁰)-Z-, -Z-S(O)ₙ—, -Z-O— (in which latter two groups, Z is attached to the carbon atom bearing R² and R³),

—N(R²⁰)C(O)O-Z-, (in which latter group, —N(R²⁰) is attached to the carbon atom bearing R² and R³) or —C(O)N(R²⁰)— (in which latter group,

—C(O) is attached to the carbon atom bearing R² and R³);

J represents C₁₋₆ alkylene optionally substituted by one or more substituents selected from —OH, halo and amino;

Z represents a direct bond or C₁₋₄ alkylene;

n represents 0, 1 or 2;

R¹⁹ and R²⁰ independently represent H or C₁₋₆ alkyl;

G represents CH or N;

R⁴ represents one or more optional substituents selected from —OH, cyano, halo, nitro, C₁₋₆ alkyl (optionally terminated by —N(H)C(O)OR^{21a}),

C₁₋₆ alkoxy, —N(R^{22a})R^{22b}, —C(O)R^{22c}, —C(O)OR^{22d}, —C(O)N(R^{22e})R^{22f}, —N(R^{22g})C(O)R^{22h}, —N(R²²ⁱ)C(O)N(R^{22j})R^{22k}, —N(R^{22m})S(O)₂R^{21b}, —S(O)₂R^{21c}, and/or—OS(O)₂R^{21d};

R^{21a} to R^{21d} independently represent C₁₋₆ alkyl;

R^{22a} and R^{22b} independently represent H, C₁₋₆ alkyl or together represent C₃₋₆ alkylene, resulting in a four- to seven-membered nitrogen-containing ring;

R^{22c} to R^{22m} independently represent H or C₁₋₆ alkyl; and

R⁴¹ to R⁴⁶ independently represent H or C₁₋₃ alkyl;

wherein each aryl and aryloxy group, unless otherwise specified, is optionally substituted;

provided that

(a) the compound is not:
3,7-dibenzoyl-9-oxa-3,7-diazabicyclo[3.3.1]nonane;

(b) when A represents -J-N(R¹⁹)— or -J-O—, then:
(i) J does not represent C₁ alkylene; and
(ii) B does not represent —N(R²⁰)—, —N(R²⁰)-Z- (in which latter group N(R²⁰) is attached to the carbon atom bearing R² and R³),

—S(O)ₙ—, —O— or —N(R²⁰)C(O)O-Z- when R² and R³ do not together represent ═O; and

(c) when R² represents —OR¹³ or —N(R¹⁴)(R¹⁵), then:
(i) A does not represent -J-N(R¹⁹)— or -J-O—; and
(ii) B does not represent —N(R²⁰)—, —N(R²⁰)-Z- (in which latter group N(R²⁰) is attached to the carbon atom bearing R² and R³),

—S(O)ₙ—, —O— or —N(R²⁰)C(O)O-Z-;

or a pharmaceutically acceptable derivative thereof.

This definition will hereinafter be referred to as a compound as defined in claim 1 of WO 01/28992. The definition of “a pharmaceutically acceptable derivative thereof” is that used in WO 01/28992 which is now repeated. Pharmaceutically acceptable derivatives include salts and solvates. Salts which may be mentioned include acid addition salts. Specific salts that may be mentioned include arylsulfonate salts, such as toluenesulfonate and, especially, benzenesulfonate salts. Solvates that may be mentioned include hydrates, such as monohydrates of the compounds of the invention.

Pharmaceutically acceptable derivatives also include, at the oxabispidine or (when G represents N) pyridyl nitrogens, C₁₋₄ alkyl quaternary ammonium salts and N-oxides, provided that when a N-oxide is present:

no Het (Het¹, Het², Het³, Het⁴, Het⁵, Het⁶, Het⁷, Het⁸, Het⁹ and Het¹⁰ group contains an unoxidised S-atom; and/or

n does not represent 0 when B represents -Z-S(O)ₙ—.

The compounds of the invention may exhibit tautomerism. All tautomeric forms and mixtures thereof are included within the scope of the invention.

Claim 34 of WO 01/28992 provides a list of compounds as follows

A compound which is:
4-[2-[7-(3,3-dimethyl-2-oxobutyl)-9-oxa-3,7-diazabicyclo[3.3.1]non-3-yl]ethyl}benzonitrile;
7-[4-(4-cyanophenyl)-4-(3,4-dimethoxyphenoxy)butyl]-N-ethyl-9-oxa-3,7-diazabicyclo[3.3.1]nonane-3-carboxamide;
4-({3-[7-(3,3-dimethyl-2-oxobutyl)-9-oxa-3,7-diazabicyclo[3.3.1]non-3-yl]propyl}amino)benzonitrile;
4-{3-[7-(4-fluorobenzyl)-9-oxa-3,7-diazabicyclo[3.3.1]non-3-yl]-2-hydroxypropoxy}benzonitrile;
4-(2-{7-[2-(4-methoxyphenyl)-2-oxoethyl]-9-oxa-3,7-diazabicyclo[3.3.1]-non-3-yl}ethoxy)benzonitrile;
4-[((2S)-2-amino-3-{7-[2-(1H-pyrrol-1-yl)ethyl]-9-oxa-3,7-diazabicyclo-[3.3.1]non-3-yl}propyl)oxy]benzonitrile;
tert-butyl 2-{7-[3-(4-cyanoanilino)propyl]-9-oxa-3,7-diazabicyclo[3.3.1]-non-3-yl}ethylcarbamate;
tert-butyl 2-{7-[4-(4-cyanophenyl)butyl]-9-oxa-3,7-diazabicyclo[3.3.1]-non-3-yl}ethylcarbamate;
tert-butyl 2-[7-[(2S)-3-(4-cyanophenoxy)-2-hydroxypropyl]-9-oxa-3,7-di-azabicyclo[3.3.1]non-3-yl}ethylcarbamate;
4-(2-{7-[4-(4-pyridinyl)butyl]-9-oxa-3,7-diazabicyclo[3.3.1]non-3-yl}-ethoxy)benzonitrile tert-butyl 2-{7-[4-(4-pyridinyl)butyl]-9-oxa-3,7-diazabicyclo[3.3.1]non-3-yl}ethylcarbamate;
4-{3-[7-(3,3-dimethyl-2-oxobutyl)-9-oxa-3,7-diazabicyclo[3.3.1]non-3-yl]-2-hydroxypropoxy}benzonitrile;
4-{3-[7-(3,4-dimethoxyphenethyl)-9-oxa-3,7-diazabicyclo[3.3.1]non-3-yl]-2-hydroxypropoxy}benzonitrile;
4-(2-[7-(3,3-dimethyl-2-oxobutyl)-9-oxa-3,7-diazabicyclo[3.3.1]non-3-yl]-ethoxy}benzonitrile;
4-({3-[7-(butylsulfonyl)-9-oxa-3,7-diazabicyclo[3.3.1]non-3-yl]propyl}-amino)benzonitrile;
4-({3-[7-(3,4-dimethoxyphenethyl)-9-oxa-3,7-diazabicyclo[3.3.1]non-3-yl]propyl}amino)benzonitrile;
4-[4-[7-(butylsulfonyl)-9-oxa-3,7-diazabicyclo[3.3.1]non-3-yl]-1-(3,4-dimethoxyphenoxy)butyl]benzonitrile;
4-{1-(3,4-dimethoxyphenoxy)-4-[7-(3,3-dimethyl-2-oxobutyl)-9-oxa-3,7-diazabicyclo[3.3.1]non-3-yl]butyl}benzonitrile;
4-[4-[7-(3,4-dimethoxyphenethyl)-9-oxa-3,7-diazabicyclo[3.3.1]non-3-yl]-1-(3,4-dimethoxyphenoxy)butyl]benzonitrile;
2-(4-acetyl-1-piperazinyl)ethyl 7-[3-(4-cyanophenoxy)-2-hydroxypropyl]-9-oxa-3,7-diazabicyclo[3.3.1]nonane-3-carboxylate;
7-[3-(4-cyanophenoxy)-2-hydroxypropyl]-N-ethyl-9-oxa-3,7-diazabicyclo-[3.3.1]nonane-3-carboxamide;
4-{3-[7-(butylsulfonyl)-9-oxa-3,7-diazabicyclo[3.3.1]non-3-yl]-2-hydroxy-propoxy}benzonitrile;
2-(4-acetyl-1-piperazinyl)ethyl 7-[2-(4-cyanophenoxy)ethyl]-9-oxa-3,7-diazabicyclo[3.3.1]nonane-3-carboxylate;
7-[2-(4-cyanophenoxy)ethyl]-N-ethyl-9-oxa-3,7-diazabicyclo[3.3.1]-nonane-3-carboxamide;
4-{2-[7-(butylsulfonyl)-9-oxa-3,7-diazabicyclo[3.3.1]non-3-yl]ethoxy}-benzonitrile;
4-{2-[7-(3,4-dimethoxyphenethyl)-9-oxa-3,7-diazabicyclo[3.3.2]non-3-yl]ethoxy}benzonitrile;
2-(4-acetyl-1-piperazinyl)ethyl 7-[3-(4-cyanoanilino)propyl]-9-oxa-3,7-diazabicyclo[3.3.1]nonane-3-carboxylate;
7-[3-(4-cyanoanilino)propyl]-N-ethyl-9-oxa-3,7-diazabicyclo[3.3.1]-nonane-3-carboxamide;
2-(4-acetyl-1-piperazinyl)ethyl 7-[4-(4-cyanophenyl)-4-(3,4-dimethoxy-phenoxy)butyl]-9-oxa-3,7-diazabicyclo[3.3.1]nonane-3-carboxylate;
4-{3-[7-(cyclopropylmethyl)-9-oxa-3,7-diazabicyclo[3.3.1]non-3-yl]-2-hydroxypropoxy}benzonitrile;
4-(3-{7-[2-(2,3-dihydro-1,4-benzodioxin-6-yl)-2-oxoethyl]-9-oxa-3,7-diazabicyclo[3.3.1]non-3-yl}-2-hydroxypropoxy)benzonitrile;
4-(3-{7-[3-(4-acetyl-1-piperazinyl)propyl]-9-oxa-3,7-diazabicyclo[3.3.1]-non-3-yl}-2-hydroxypropoxy)benzonitrile;
2-{7-[3-(4-cyanophenoxy)-2-hydroxypropyl]-9-oxa-3,7-diazabicyclo-[3.3.1]non-3-yl}-N-isopropylacetamide;
4-(3-{7-[3-(ethylsulfonyl)propyl]-9-oxa-3,7-diazabicyclo[3.3.1]non-3-yl}-2-hydroxypropoxy)benzonitrile;
4-(2-hydroxy-3-{7-[2-(2-methoxyethoxy)ethyl]-9-oxa-3,7-diazabicyclo-[3.3.1]non-3-yl}propoxy)benzonitrile;
4-(2-hydroxy-3-{7-[2-(4-methoxyphenyl)-2-oxoethyl]-9-oxa-3,7-diaza-bicyclo[3.3.1]non-3-yl}propoxy)benzonitrile;
4-({3-[7-(cyclopropylmethyl)-9-oxa-3,7-diazabicyclo[3.3.1]non-3-yl]-propyl}amino)benzonitrile;
4-[(3-{7-[2-(2,3-dihydro-1,4-benzodioxin-6-yl)-2-oxoethyl]-9-oxa-3,7-diazabicyclo[3.3.1]non-3-yl}propyl)amino]benzonitrile;
4-[(3-{7-[2-(4-methyl-1,3-thiazol-5-yl)ethyl]-9-oxa-3,7-diazabicyclo-[3.3.1]non-3-yl}propyl)amino]benzonitrile;
4-[(3-(7-[3-(4-acetyl-1-piperazinyl)propyl]-9-oxa-3,7-diazabicyclo[3.3.1]-non-3-yl)propyl)amino]benzonitrile;
2-{7-[3-(4-cyanoanilino)propyl]-9-oxa-3,7-diazabicyclo[3.3.1]non-3-yl}-N-isopropylacetamide;
4-[(3-{7-[3-(ethylsulfonyl)propyl]-9-oxa-3,7-diazabicyclo[3.3.1]non-3-yl}-propyl)amino]benzonitrile;
4-[(3-(7-[2-(2-methoxyethoxy)ethyl]-9-oxa-3,7-diazabicyclo[3.3.1]non-3-yl}propyl)amino]benzonitrile;
4-({3-[7-(4-fluorobenzyl)-9-oxa-3,7-diazabicyclo[3.3.1]non-3-yl]propyl}-amino)benzonitrile;
4-[(3-{7-[2-(4-methoxyphenyl)-2-oxoethyl]-9-oxa-3,7-diazabicyclo[3.3.1]-non-3-yl}propyl)amino]benzonitrile;
4-{2-[7-(cyclopropylmethyl)-9-oxa-3,7-diazabicyclo[3.3.1]non-3-yl]-ethoxy}benzonitrile;
4-(2-{7-[2-(2,3-dihydro-1,4-benzodioxin-6-yl)-2-oxoethyl]-9-oxa-3,7-diazabicyclo[3.3.1]non-3-yl}ethoxy)benzonitrile;
4-(2-{7-[2-(4-methyl-1,3-thiazol-5-yl)ethyl]-9-oxa-3,7-diazabicyclo[3.3.1]-non-3-yl}ethoxy)benzonitrile;
4-(2-{7-[3-(4-acetyl-1-piperazinyl)propyl]-9-oxa-3,7-diazabicyclo[3.3.1]-non-3-yl}ethoxy)benzonitrile;
2-{7-[2-(4-cyanophenoxy)ethyl]-9-oxa-3,7-diazabicyclo[3.3.1]non-3-yl}-N-isopropylacetamide;
4-(2-{7-[3-(ethylsulfonyl)propyl]-9-oxa-3,7-diazabicyclo[3.3.1]non-3-yl}-ethoxy)benzonitrile;
4-(2-{7-[2-(2-methoxyethoxy)ethyl]-9-oxa-3,7-diazabicyclo[3.3.1]non-3-yl}ethoxy)benzonitrile;
4-{2-[7-(4-fluorobenzyl)-9-oxa-3,7-diazabicyclo[3.3.1]non-3-yl]ethoxy}-benzonitrile;
4-({3-[7-(3,3-dimethyl-2-oxobutyl)-9-oxa-3,7-diazabicyclo[3.3.1]non-3-yl]propyl}sulfonyl)benzonitrile;
4-({3-[7-(cyclopropylmethyl)-9-oxa-3,7-diazabicyclo[3.3.1]non-3-yl]-propyl}sulfonyl)benzonitrile;
4-[(3-(7-[2-(2,3-dihydro-1,4-benzodioxin-6-yl)-2-oxoethyl]-9-oxa-3,7-diazabicyclo[3.3.1]non-3-yl}propyl)sulfonyl]benzonitrile;
4-[(3-{7-[2-(4-methyl-1,3-thiazol-5-yl)ethyl]-9-oxa-3,7-diazabicyclo-[3.3.1]non-3-yl}propyl)sulfonyl]benzonitrile;
4-[(3-(7-[3-(4-acetyl-1-piperazinyl)propyl]-9-oxa-3,7-diazabicyclo[3.3.1]-non-3-yl}propyl)sulfonyl]benzonitrile;
2-(7-{3-[(4-cyanophenyl)sulfonyl]propyl}-9-oxa-3,7-diazabicyclo[3.3.1]-non-3-yl)-N-isopropylacetamide;
4-[(3-{7-[3-(ethylsulfonyl)propyl]-9-oxa-3,7-diazabicyclo[3.3.1]non-3-yl}-propyl)sulfonyl]benzonitrile;
4-[(3-{7-[2-(2-methoxyethoxy)ethyl]-9-oxa-3,7-diazabicyclo[3.3.1]non-3-yl}propyl)sulfonyl]benzonitrile;
4-({3-[7-(4-fluorobenzyl)-9-oxa-3,7-diazabicyclo[3.3.1]non-3-yl]propyl}-sulfonyl)benzonitrile;
4-[(3-{7-[2-(4-methoxyphenyl)-2-oxoethyl]-9-oxa-3,7-diazabicyclo[3.3.1]-non-3-yl}propyl)sulfonyl]benzonitrile;
4-[(3-{7-[2-(4-fluorophenyl)-2-oxoethyl]-9-oxa-3,7-diazabicyclo[3.3.1-non-3-yl}propyl)amino]benzonitrile;
4-(2-{7-[2-(4-fluorophenyl)-2-oxoethyl]-9-oxa-3,7-diazabicyclo[3.3.1]non-3-yl}ethoxy)benzonitrile;
4-{2-[7-(tetrahydro-2H-pyran-2-ylmethyl)-9-oxa-3,7-diazabicyclo[3.3.1]-non-3-yl]ethoxy}benzonitrile;
4-(3-{7-[2-(4-fluorophenyl)-2-oxoethyl]-9-oxa-3,7-diazabicyclo[3.3.1]non-3-yl}-2-hydroxypropoxy)benzonitrile;
4-{2-hydroxy-3-[7-(tetrahydro-2H-pyran-2-ylmethyl)-9-oxa-3,7-diaza-bicyclo[3.3.1]non-3-yl]propoxy}benzonitrile;
4-({3-[7-(2-fluoro-3,3-dimethylbutyl)-9-oxa-3,7-diazabicyclo[3.3.1]non-3-yl]propyl}amino)benzonitrile;
4-({3-[7-(2-hydroxy-3,3-dimethylbutyl)-9-oxa-3,7-diazabicyclo[3.3.1]non-3-yl]propyl}amino)benzonitrile;
4-({3-[7-(3,3-dimethylbutyl)-9-oxa-3,7-diazabicyclo[3.3.1]non-3-yl]-propyl}amino)benzonitrile;
4-({3-[7-(2-oxopropyl)-9-oxa-3,7-diazabicyclo[3.3.1]non-3-yl]propyl}-amino)benzonitrile;
4-(2-{7-[3-(4-cyanoanilino)propyl]-9-oxa-3,7-diazabicyclo[3.3.1]non-3-yl}ethoxy)benzonitrile;
4-(2-{7-[2-(4-cyanophenoxy)ethyl]-9-oxa-3,7-diazabicyclo[3.3.1]non-3-yl}ethoxy)benzonitrile;
4-(2-{7-[2-(4-cyanophenoxy)ethyl]-9-oxa-3,7-diazabicyclo[3.3.1]non-3-yl}ethyl)benzonitrile;
4-{4-[7-(3,3-dimethyl-2-oxobutyl)-9-oxa-3,7-diazabicyclo[3.3.1]non-3-yl]-butyl}benzonitrile;
4-{2-[7-(2-phenoxyethyl)-9-oxa-3,7-diazabicyclo[3.3.1]non-3-yl]ethoxy}-benzonitrile;
2-{7-[2-(4-cyanophenoxy)ethyl]-9-oxa-3,7-diazabicyclo[3.3.1]non-3-yl}-N,N-diethylacetamide;
4-[(3-{7-[4-(4-fluorophenyl)-4-oxobutyl]-9-oxa-3,7-diazabicyclo[3.3.1]-non-3-yl}propyl)amino]benzonitrile;
4-({7-[3-(4-cyanoanilino)propyl]-9-oxa-3,7-diazabicyclo[3.3.1]non-3-yl}-methyl)benzonitrile;
4-{2-[7-(2,4-difluorobenzyl)-9-oxa-3,7-diazabicyclo[3.3.1]non-3-yl]-ethoxy}benzonitrile;
4-[(3-{7-[4-(difluoromethoxy)benzyl]-9-oxa-3,7-diazabicyclo[3.3.1]non-3-yl}propyl)amino]benzonitrile;
4-[(3-{7-[2-(1H-pyrrol-1-yl)ethyl]-9-oxa-3,7-diazabicyclo[3.3.1]non-3-yl}-propyl)amino]benzonitrile;
4-[(3-{7-[3-(4-bromophenyl)-3-oxopropyl]-9-oxa-3,7-diazabicyclo[3.3.1]-non-3-yl}propyl)amino]benzonitrile;
4-{2-[7-(2,2-difluoroethyl)-9-oxa-3,7-diazabicyclo[3.3.1]non-3-yl]ethoxy}-benzonitrile;
4-({3-[7-(2-phenoxyethyl)-9-oxa-3,7-diazabicyclo[3.3.1]non-3-yl]propyl}-amino)benzonitrile;
4-(2-{7-[2-(1H-pyrrol-1-yl)ethyl]-9-oxa-3,7-diazabicyclo[3.3.1]non-3-yl}-ethoxy)benzonitrile;
4-[((2S)-3-{7-[(2S)-3-(4-cyanophenoxy)-2-hydroxypropyl]-9-oxa-3,7-diazabicyclo[3.3.1]non-3-yl}-2-hydroxypropyl)oxy]benzonitrile;
4-[((2S)-2-hydroxy-3-{7-[2-(1H-pyrrol-1-yl)ethyl]-9-oxa-3,7-diazabicyclo-[3.3.1]non-3-yl}propyl)oxy]benzonitrile;
4-{2-[7-(3,3-dimethyl-2-oxobutyl)-9-oxa-3,7-diazabicyclo[3.3.1]non-3-yl]-ethoxy}isophthalonitrile;
4-(2-{7-[2-(4-methoxyphenyl)-2-oxoethyl]-9-oxa-3,7-diazabicyclo[3.3.1]-non-3-yl}ethoxy)isophthalonitrile;
4-(2-{7-[2-(1H-pyrrol-1-yl)ethyl]-9-oxa-3,7-diazabicyclo[3.3.1]non-3-yl}-ethoxy)isophthalonitrile;
tert-butyl 2-{7-[2-(2,4-dicyanophenoxy)ethyl]-9-oxa-3,7-diazabicyclo-[3.3.1]non-3-yl}ethylcarbamate;
4-({(2S)-2-amino-3-[7-(3,3-dimethyl-2-oxobutyl)-9-oxa-3,7-diazabicyclo-[3.3.1non-3-yl]propyl}oxy)benzonitrile;
4-[((2S)-2-amino-3-{7-[2-(4-methoxyphenyl)-2-oxoethyl]-9-oxa-3,7-diaza-bicyclo[3.3.1]non-3-yl}propyl)oxy]benzonitrile;
4-{3-[7-(3,3-dimethyl-2-oxobutyl)-9-oxa-3,7-diazabicyclo[3.3.1]non-3-yl]-propoxy}benzonitrile;
4-(3-{7-[2-(4-fluorophenyl)-2-oxoethyl]-9-oxa-3,7-diazabicyclo[3.3.1]non-3-yl}propoxy)benzonitrile;
4-(3-{7-[2-(1H-pyrrol-1-yl)ethyl]-9-oxa-3,7-diazabicyclo[3.3.1]non-3-yl}-propoxy)benzonitrile;
4-(4-{7-[2-(1H-pyrrol-1-yl)ethyl]-9-oxa-3,7-diazabicyclo[3.3.1]non-3-yl}-butyl)benzonitrile;
4-{[(2S)-3-(7-{2-[4-(tert-butoxy)phenoxy]ethyl}-9-oxa-3,7-diazabicyclo-[3.3.1]non-3-yl)-2-hydroxypropyl]oxy}benzonitrile;
4-[((2S)-3-{7-[2-(3,5-dimethyl-1H-pyrazol-1-yl)ethyl]-9-oxa-3,7-diaza-bicyclo[3.3.1]non-3-yl}-2-hydroxypropyl)oxy]benzonitrile;
4-{3-[7-(imidazo[1,2-a]pyridin-2-ylmethyl)-9-oxa-3,7-diazabicyclo[3.3.1]-non-3-yl]propoxy}benzonitrile;
4-{3-[7-(2-phenoxyethyl)-9-oxa-3,7-diazabicyclo[3.3.1]non-3-yl]propoxy}-benzonitrile;
4-(3-{7-[2-(3,5-dimethyl-1H-pyrazol-1-yl)ethyl]-9-oxa-3,7-diazabicyclo-[3.3.1]non-3-yl}propoxy)benzonitrile;
4-({3-[7-(imidazo[1,2-a]pyridin-2-ylmethyl)-9-oxa-3,7-diazabicyclo-[3.3.1]non-3-yl]propyl}amino)benzonitrile;
4-({3-[7-(2,4-difluorobenzyl)-9-oxa-3,7-diazabicyclo[3.3.1]non-3-yl]-propyl}amino)benzonitrile;
4-{[3-(7-{2-[4-(tert-butoxy)phenoxy]ethyl}-9-oxa-3,7-diazabicyclo[3.3.1]-non-3-yl)propyl]amino}benzonitrile;
4-{2-[7-(imidazo[1,2-a]pyridin-2-ylmethyl)-9-oxa-3,7-diazabicyclo[3.3.1]-non-3-yl]ethoxy}benzonitrile;
tert-butyl 2-{7-[2-(4-cyanophenoxy)ethyl]-9-oxa-3,7-diazabicyclo[3.3.1]-non-3-yl}ethylcarbamate;
4-{[3-(7-{2-[4-(tert-butoxy)phenoxy]ethyl)-9-oxa-3,7-diazabicyclo[3.3.1]-non-3-yl)propyl]sulfonyl}benzonitrile;
4-[(3-{7-[2-(3,5-dimethyl-1H-pyrazol-1-yl)ethyl]-9-oxa-3,7-diazabicyclo-[3.3.1]non-3-yl}propyl)sulfonyl]benzonitrile;
4-({3-[7-(2,4-difluorobenzyl)-9-oxa-3,7-diazabicyclo[3.3.1]non-3-yl]-propyl}sulfonyl)benzonitrile;
4-{2-[7-(imidazo[1,2-a]pyridin-2-ylmethyl)-9-oxa-3,7-diazabicyclo[3.3.1]-non-3-yl]ethoxy}isophthalonitrile;
4-[2-(7-{2-[4-(tert-butoxy)phenoxy]ethyl}-9-oxa-3,7-diazabicyclo[3.3.1]-non-3-yl)ethoxy]isophthalonitrile;
4-(2-{7-[2-(3,5-dimethyl-1H-pyrazol-1-yl)ethyl]-9-oxa-3,7-diazabicyclo-[3.3.1]non-3-yl}ethoxy)isophthalonitrile;
4-(4-{7-[2-(1H-imidazol-4-yl)ethyl]-9-oxa-3,7-diazabicyclo[3.3.1]non-3-yl}butyl)benzonitrile;
4-{4-[7-(imidazo[1,2-a]pyridin-2-ylmethyl)-9-oxa-3,7-diazabicyclo[3.3.1]-non-3-yl]butyl}benzonitrile;
4-{4-[7-(2-phenoxyethyl)-9-oxa-3,7-diazabicyclo[3.3.1]non-3-yl]butyl}-benzonitrile;
4-(4-{7-[2-(3,5-dimethyl-1H-pyrazol-1-yl)ethyl]-9-oxa-3,7-diazabicyclo-[3.3.1]non-3-yl}butyl)benzonitrile;
4-[3-(7-{2-oxo-2-[4-(1-pyrrolidinyl)phenyl]ethyl}-9-oxa-3,7-diazabicyclo-[3.3.1πnon-3-yl)propoxy]benzonitrile;
4-(3-{7-[2-(4-hydroxyphenyl)-2-oxoethyl]-9-oxa-3,7-diazabicyclo[3.3.1]-non-3-yl}propoxy)benzonitrile;
4-(3-{7-[2-(4-methylphenyl)-2-oxoethyl]-9-oxa-3,7-diazabicyclo[3.3.1]-non-3-yl}propoxy)benzonitrile;
4-(3-{7-[2-(4-methoxyphenyl)-2-oxoethyl]-9-oxa-3,7-diazabicyclo[3.3.1]-non-3-yl}propoxy)benzonitrile;
4-(3-{7-[2-(2,3-dihydro-1,4-benzodioxin-6-yl)-2-oxoethyl]-9-oxa-3,7-di-azabicyclo[3.3.1]non-3-yl}propoxy)benzonitrile;
4-(2-{7-[2-(2,6-dimethylphenoxy)-1-methylethyl]-9-oxa-3,7-diazabicyclo-[3.3.1]non-3-yl}ethoxy)benzonitrile;
4-(3-{7-[2-oxo-2-(3-oxo-3,4-dihydro-2H-1,4-benzoxazin-6-yl)ethyl]-9-3,7-diazabicyclo[3.3.1]non-3-yl}propoxy)benzonitrile;
tert-butyl 2-{7-[3-(4-cyanophenoxy)propyl]-9-oxa-3,7-diazabicyclo[3.3.1]-non-3-yl}ethylcarbamate;
N-(tert-butyl)-N′-(2-{7-[3-(4-cyanophenoxy)propyl]-9-oxa-3,7-diazabi-cyclo[3.3.1]non-3-yl}ethyl)urea;
tert-butyl 2-({7-[2-(4-cyanophenoxy)ethyl]-9-oxa-3,7-diazabicyclo[3.3.1]-non-3-yl}methyl)-1-pyrrolidinecarboxylate;
4-{[3-(7-benzyl-9-oxa-3,7-diazabicyclo[3.3.1]non-3-yl)propyl]amino}-benzonitrile;
4-[(3-{7-[3-(4-cyanoanilino)propyl]-9-oxa-3,7-diazabicyclo[3.3.1]non-3-yl}propyl)amino]benzonitrile;
tert-butyl 2-{7-[2-(4-nitrophenoxy)ethyl]-9-oxa-3,7-diazabicyclo[3.3.1]-non-3-yl}ethylcarbamate (m/z=437);
tert-butyl 2-[7-(2-{4-[(methylsulfonyl)amino]phenoxy}ethyl)-9-oxa-3,7-diazabicyclo[3.3.1]non-3-yl]ethylcarbamate;
tert-butyl 2-{7-[2-(4-aminophenoxy)ethyl]-9-oxa-3,7-diazabicyclo[3.3.1]-non-3-yl}ethylcarbamate;
4-({3-[7-(phenylsulfonyl)-9-oxa-3,7-diazabicyclo[3.3.1]non-3-yl]propyl}-amino)benzonitrile; or
4-({3-[7-(3,3-dimethyl-2-oxobutyl)-9-oxa-3,7-diazabicyclo[3.3.1]non-3-yl]propyl}amino)benzamide.

This list of compounds and including pharmaceutically acceptable derivatives of the compounds as defined in WO 01/28992 will hereinafter be referred to as a compound of claim 34 of WO 01/28992.

PCT/SE02/00724 discloses modified release formulations of the following compounds which are described in WO 01/28992:

(a) 4-({3-[7-(3,3-dimethyl-2-oxobutyl)-9-oxa-3,7-diazabicyclo[3.3.1]non-3-yl]propyl}amino)benzonitrile: 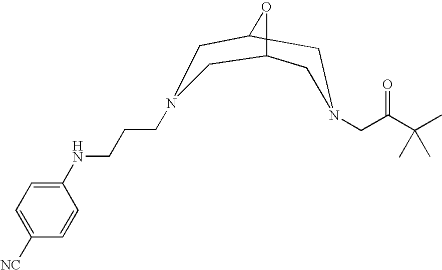
which compound is referred to hereinafter as Compound A. Compound A is specifically disclosed in WO 01/28992 both in the form of the free base and in the form of a benzenesulphonate salt;

(b) tert-butyl 2-{7-[3-(4-cyanoanilino)propyl]-9-oxa-3,7-diazabicyclo-[3.3.1]non-3-yl}ethylcarbamate: 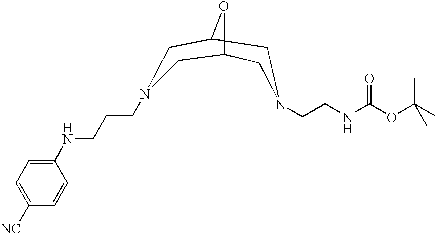
in the form of the free base, which compound is referred to hereinafter as Compound B;

(c) tert-butyl 2-{7-[4-(4-cyanophenyl)butyl]-9-oxa-3,7-diazabicyclo-[3.3.1]non-3-yl}ethylcarbamate: 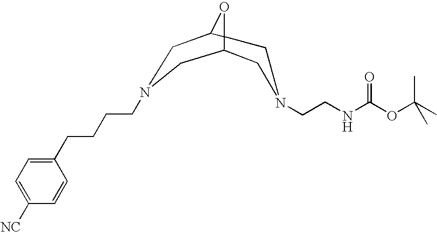
in the form of the free base, which compound is referred to hereinafter as Compound C; and

(d) tert-butyl 2-{7-[(2S)-3-(4-cyanophenoxy)-2-hydroxypropyl]-9-oxa-3,7-diazabicyclo[3.3.1]non-3-yl}ethylcarbamate: 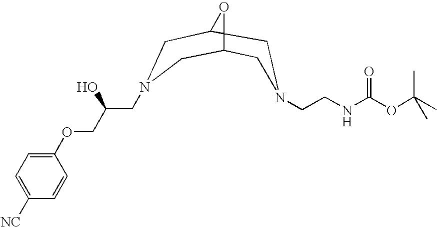
in the form of the free base, which compound is referred to hereinafter as Compound D.

Current drug therapies for AF include antiarryhthmic drugs, administered with a view to re-establishing and maintaining a normal heartbeat or to controlling heart rate, and anticoagulant and/or thrombolytic drugs, administered with a view to preventing thromboembolism and/or cerebral stroke.

Coagulation is the result of a complex series of enzymatic reactions. One of the ultimate steps in this series of reactions is the conversion of the proenzyme prothrombin to the active enzyme thrombin.

Thrombin is known to play a central role in coagulation. It activates platelets, leading to platelet aggregation, converts fibrinogen into fibrin monomers, which polymerise spontaneously into fibrin polymers, and activates factor XIII, which in turn crosslinks the polymers to form insoluble fibrin. Furthermore, thrombin activates factor V and factor VIII leading to a “positive feedback” generation of thrombin from prothrombin.

International patent application WO 02/44145 discloses thrombin-inhibiting compounds of formula 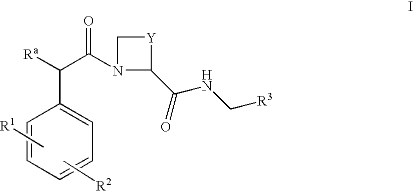
wherein

R^{a} represents —OH or —CH₂OH;

R¹ represents at least one optional halo substituent;

R² represents one or two C₁₋₃ alkoxy substituents, the alkyl parts of which substituents are themselves substituted with one or more fluoro substituents (i.e. R² represents one or two fluoroalkoxy(C₁₋₃) groups);

Y represents —CH₂— or —(CH₂)₂—; and

R³ represents a structural fragment of formula I(i) or I(ii): 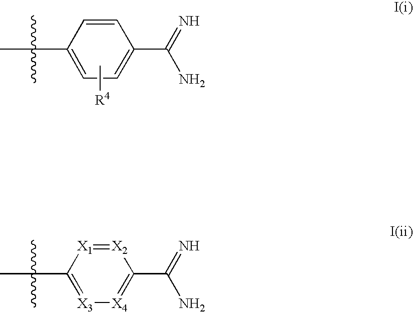
wherein

R⁴ represents H or one or more fluoro substituents; and one or two of X₁, X₂, X₃ and X₄ represent —N— and the others represent —CH—,

or a pharmaceutically-acceptable derivative thereof in claim 1. Such compounds are hereinafter referred to as a compound of claim 1 in WO 02/44145.

Claim 20 of WO 02/44145 discloses the following compounds:
Ph(3-Cl)(5-OCHF₂)—(R)CH(OH)C(O)-Aze-Pab;
Ph(3-Cl)(5-OCF₃)—(R)CH(OH)C(O)-Aze-Pab;
Ph(3-Cl)(5-OCHF₂)—(S)CH(CH₂OH)C(O)-Aze-Pab;
Ph(3-Cl)(5-OCF₃)—(S)CH(CH₂OH)C(O)-Aze-Pab;
Ph(3-OCHF₂)—(R)CH(OH)—CO-Aze-Pab;
Ph(3-OCF₃)—(R)CH(OH)—CO-Aze-Pab;
Ph(3-Cl)(5-OCH₂CF₃)—(R)CH(OH)C(O)-Aze-Pab;
Ph(3-Cl)(5-OCH₂CHF₂)—(R)CH(OH)C(O)-Aze-Pab;
Ph(3-Cl)(5-OCH₂F)—(R)CH(OH)C(O)-Aze-Pab;
Ph(3-Cl)(5-OCH₂CH₂F)—(R)CH(OH)C(O)-Aze-Pab;
Ph(3-Cl)(5-OCH(CH₂F)₂)—(R)CH(OH)C(O)-Aze-Pab;
Ph(3-F)(5-OCHF₂)—(R)CH(OH)C(O)-Aze-Pab;
Ph(3-Br)(5-OCH₂F)—(R)CH(OH)C(O)-Aze-Pab;
Ph(3-Br)(5-OCHF₂)—(R)CH(OH)C(O)-Aze-Pab;
Ph(3-Cl, 5-OCHF₂)—(R)CH(OH)C(O)-Pro-Pab;
Ph(3-Cl, 5-OCHF₂)—(R)CH(OH)C(O)-Aze-NH—CH₂-((2-amidino)-5-pyridinyl);
Ph(3-Cl, 5-OCHF₂)—(R)CH(OH)C(O)-Aze-NH—CH₂-((5-amidino)-2-pyrimidinyl);
Ph(3-Cl, 5-OCHF₂)—(R)CH(OH)C(O)-Aze-Pab(3-F);
Ph(3-Cl, 5-OCHF₂)—(R)CH(OH)C(O)-Aze-Pab(2,6-diF);
Ph(3-Cl, 5-OCHF₂)—(R)CH(OH)C(O)-Aze-Pab(2,5-diF).
Ph(3-Cl)(5-OCHF₂)—(R)CH(OH)C(O)-Aze-Pab(OMe);
Ph(3-Cl)(5-OCHF₂)—(R)CH(OH)C(O)-Aze-Pab(OEt);
Ph(3-Cl)(5-OCHF₂)—(R)CH(OH)C(O)-Aze-Pab(OnPr);
Ph(3-Cl)(5-OCHF₂)—(R)CH(OH)C(O)-Aze-Pab(OiPr);
Ph(3-Cl)(5-OCHF₂)—(R)CH(OH)C(O)-Aze-Pab(OcBu);
Ph(3-Cl)(5-OCHF₂)—(R)CH(OH)C(O)-Aze-Pab(OH);
Ph(3-Cl)(5-OCHF₂)—(R)CH(OH)C(O)-Aze-Pab(COOcPentyl);
Ph(3-Cl)(5-OCHF₂)—(R)CH(OH)C(O)-Aze-Pab(Z);
Ph(3-Cl)(5-OCF₃)—(R)CH(OH)C(O)-Aze-Pab(OMe);
Ph(3-Cl)(5-OCF₃)—(R)CH(OH)C(O)-Aze-Pab(OCH₂-3-(5-Me-isoxazole));
Ph(3-Cl)(5-OCF₃)—(R)CH(OH)C(O)-Aze-Pab(OCH₂-3-pyridine);
Ph(3-Cl)(5-OCF₃)—(R)CH(OH)C(O)-Aze-Pab(OiBu);
Ph(3-Cl)(5-OCF₃)—(R)CH(OH)C(O)-Aze-Pab(OEt);
Ph(3-Cl)(5-OCF₃)—(R)CH(OH)C(O)-Aze-Pab(OBn);
Ph(3-Cl)(5-OCF₃)—(R)CH(OH)C(O)-Aze-Pab(OcHexyl);
Ph(3-Cl)(5-OCF₃)—(R)CH(OH)C(O)-Aze-Pab(OcBu);
Ph(3-Cl)(5-OCF₃)—(R)CH(OH)C(O)-Aze-Pab(OCH₂CH₂OPh(3-CF₃));
Ph(3-Cl)(5-OCF₃)—(R)CH(OH)C(O)-Aze-Pab(OBn(4-Cl));
Ph(3-Cl)(5-OCF₃)—(R)CH(OH)C(O)-Aze-Pab(OBn(3-MeO));
Ph(3-Cl)(5-OCF₃)—(R)CH(OH)C(O)-Aze-Pab(OBn(2-Br));
Ph(3-Cl)(5-OCF₃)—(R)CH(OH)C(O)-Aze-Pab(OBn(4-Me));
Ph(3-Cl)(5-OCF₃)—(R)CH(OH)C(O)-Aze-Pab(O-4-heptyl);
Ph(3-Cl)(5-OCF₃)—(S)CH(CH₂OH)C(O)-Aze-Pab(OMe);
Ph(3-Cl)(5-OCH₂CF₃)—(R)CH(OH)C(O)-Aze-Pab(OMe);
Ph(3-Cl)(5-OCH₂CHF₂)—(R)CH(OH)C(O)-Aze-Pab(OMe);
Ph(3-Cl)(5-OCH₂F)—(R)CH(OH)C(O)-Aze-Pab(OMe);
Ph(3-Cl)(5-OCH₂CH₂F)—(R)CH(OH)C(O)-Aze-Pab(OMe);
Ph(3-Cl)(5-OCH(CH₂F)₂)—(R)CH(OH)C(O)-Aze-Pab(OMe);
Ph(3-F)(5-OCHF₂)—(R)CH(OH)C(O)-Aze-Pab(OMe);
Ph(3-Br)(5-OCHF₂)—(R)CH(OH)C(O)-Aze-Pab(OMe);
Ph(3-Cl, 5-OCH₂CHF₂)—(R)CH(OH)C(O)-Aze-Pab(OH);
Ph(3-Cl, 5-OCH₂CH₂F)—(R)CH(OH)C(O)-Aze-Pab(OH);
Ph(3-Cl, 5-OCHF₂)—(R)CH(OH)C(O)-Pro-Pab(OMe);
Ph(3-Cl, 5-OCHF₂)—(R)CH(OH)C(O)-Aze-NH—CH₂-((2-methoxy-amidino)-5-pyridinyl);
Ph(3-Cl, 5-OCHF₂)—(R)CH(OH)C(O)-Aze-NH—CH₂-((5-methoxy-amidino)-2-pyrimidinyl);
Ph(3-Cl, 5-OCHF₂)—(R)CH(OH)C(O)-Aze-Pab(2,6-diF)(OMe); or
Ph(3-Cl, 5-OCHF₂)—(R)CH(OH)C(O)-Aze-Pab(2,5-diF)(OMe).

Such compounds and pharmaceutically-acceptable derivatives of these compounds are hereinafter referred to as a compound of claim 20 in WO 02/44145.

The following compounds represent sub-set I of the compounds of claim 20 of WO 02/44145:
Ph(3-Cl)(5-OCHF₂)—(R)CH(OH)C(O)—(S)Aze-Pab(OMe);
Ph(3-Cl)(5-OCHF₂)—(R)CH(OH)C(O)—(S)Aze-Pab(2,6-diF)(OMe);
Ph(3-Cl)(5-OCH₂CH₂F)—(R)CH(OH)C(O)—(S)Aze-Pab(OMe);
Ph(3-Cl)(5-OCHF₂)—(R)CH(OH)C(O)—(S)Aze-Pab;
Ph(3-Cl)(5-OCHF₂)—(R)CH(OH)C(O)—(S)Aze-Pab(OH);
Ph(3-Cl)(5-OCHF₂)—(R)CH(OH)C(O)—(S)Aze-Pab(2,6-diF);
Ph(3-Cl)(5-OCH₂CH₂F)—(R)CH(OH)C(O)—(S)Aze-Pab; or,
Ph(3-Cl)(5-OCH₂CH₂F)—(R)CH(OH)C(O)—(S)Aze-Pab(OH).

The following compounds represent sub-set 2 of the compounds of claim 20 of WO 02/44145:
Ph(3-Cl)(5-OCHF₂)—(R)CH(OH)C(O)—(S)Aze-Pab(2,6-diF)(OMe);
Ph(3-Cl)(5-OCH₂CH₂F)—(R)CH(OH)C(O)—(S)Aze-Pab(OMe); or
Ph(3-Cl)(5-OCHF₂)—(R)CH(OH)C(O)—(S)Aze-Pab.

The following compound represent sub-set 3 of the compounds of claim 20 of WO 02/44145:
Ph(3-Cl)(5-OCHF₂)—(R)CH(OH)C(O)—(S)Aze-Pab(2,6-diF)(OMe).

Combinations of a compound from any one of sub-sets 1, 2 and 3 and a compound A, B, C or D are particular combinations of the present invention.

The term “pharmaceutically-acceptable derivatives” in WO 02/44145 includes pharmaceutically-acceptable salts (e.g. acid addition salts).

In WO 02/44145 pharmaceutically acceptable derivatives of compounds of formula I also include “protected” derivatives, and/or compounds that act as prodrugs, of compounds of formula I.

Compounds that may act as prodrugs of compounds of formula I that may be mentioned include compounds of formula Ia, 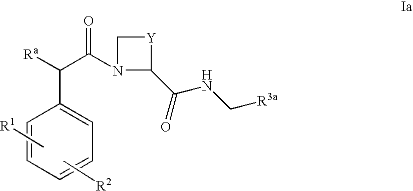
wherein R^{3a} represents a structural fragment of formula I(iii) or I(iv): 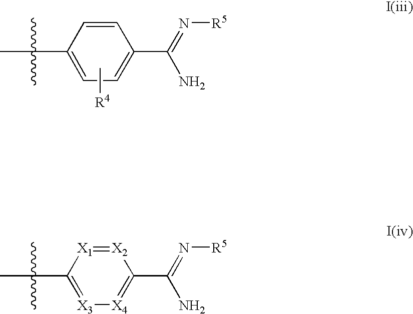
wherein R⁵ represents OR⁶ or C(O)OR⁷;

R⁶ represents H, C₁₋₁₀ alkyl, C₁₋₃ alkylaryl or C₁₋₃ alkyloxyaryl (the alkyl parts of which latter two groups are optionally interrupted by one or more oxygen atoms, and the aryl parts of which latter two groups are optionally substituted by one or more substituents selected from halo, phenyl, methyl or methoxy, which latter three groups are also optionally substituted by one or more halo substituents);

R⁷ represents C₁₋₁₀ alkyl (which latter group is optionally interrupted by one or more oxygen atoms), or C₁₋₃ alkylaryl or C₁₋₃ alkyloxyaryl (the alkyl parts of which latter two groups are optionally interrupted by one or more oxygen atoms, and the aryl parts of which latter two groups are optionally substituted by one or more substituents selected from halo, phenyl, methyl or methoxy, which latter three groups are also optionally substituted by one or more halo substituents); and

R^{a}, R¹, R², Y, R⁴, X₁, X₂, X₃ and X₄ are as hereinbefore defined, and pharmaceutically-acceptable derivatives thereof.

The term “pharmaceutically-acceptable derivatives” of compounds of formula Ia includes pharmaceutically-acceptable salts (e.g. acid addition salts).

The wavy lines on the bonds in the fragments of formulae I(iii) and I(iv) signify the bond positions of the fragments.

In particular, compounds of the invention are potent inhibitors of thrombin either as such and/or (e.g. in the case of prodrugs), are metabolised following administration to form potent inhibitors of thrombin, for example as may be demonstrated in the tests described below.

By “prodrug of a thrombin inhibitor”, we include compounds that form a thrombin inhibitor, in an experimentally-detectable amount, and within a predetermined time (e.g. about 1 hour), following oral or parenteral administration (see, for example, Test E below) or, alternatively, following incubation in the presence of liver microsomes (see, for example, Test G below).

However, it is estimated that only 40% of patients with AF who should benefit from anticoagulant therapy do so, owing to the risks associated with existing treatments. This also includes patients whose anticoagulant therapy is in combination with cardioversion (electrical or chemical). In particular, of the currently-available oral anticoagulants, warfarin (a vitamin K antagonist) carries the risk of bleeding, and the need for frequent laboratory control. Vitamin K antagonists also demonstrate a notable risk of interaction with other drugs and certain foods, e.g. those that are rich in Vitamin K, and their use requires monitoring of the patient's blood coagulation status. Medication containing acetylsalicylic acid (an antiplatelet agent) also carries the risk of bleeding. Blood coagulation is the key process involved in both haemostasis (i.e. the prevention of blood loss from a damaged vessel) and thrombosis (i.e. the formation of a blood clot in a blood vessel, sometimes leading to vessel obstruction).

There remains a need for a combination of an antiarrhythmic drug and an anti-coagulant drug that has fewer side-effects than existing therapies and will encourage the use of such a combination in a higher percentage of AF patients, thus reducing morbidity and mortality in this patient group.

None of the above-mentioned documents disclose or suggest the administration of a compound of claim 1 in WO 02/44145 in conjunction with a compound as defined in claim 1 of WO 01/28992. Surprisingly, the administration of just such a combination gives rise to unexpected, beneficial effects.

### DISCLOSURE OF THE INVENTION

According to a first aspect of the invention there is provided a combination product comprising:

(1) a compound of claim 1 in WO 02/44145; and

(2) a compound as defined in claim 1 of WO 01/28992.

According to a second aspect of the invention there is provided a combination product comprising:

(1) a compound of claim 20 in WO 02/44145; and

(2) a compound of claim 34 of WO 01/28992.

According to a third aspect of the invention there is provided a combination product comprising:

(1) a compound of claim 20 in WO 02/44145; and

(2) (a) 4-({3-[7-(3,3-dimethyl-2-oxobutyl)-9-oxa-3,7-diazabicyclo[3.3.1]non-3-yl]propyl}amino)benzonitrile: 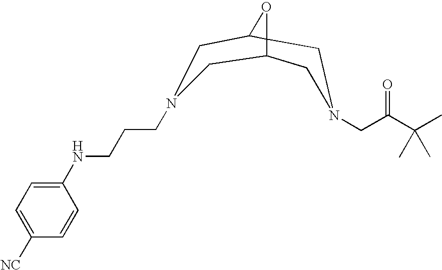
which compound is referred to hereinafter as Compound A or a pharmaceutically-acceptable salt thereof; or

(b) tert-butyl 2-{7-[3-(4-cyanoanilino)propyl]-9-oxa-3,7-diazabicyclo-[3.3.1]non-3-yl}ethylcarbamate: 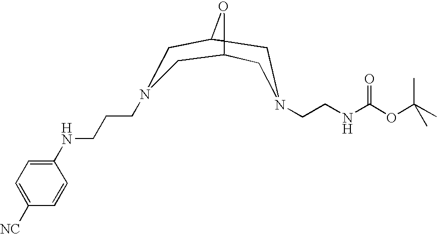
in the form of the free base, which compound is referred to hereinafter as Compound B or a pharmaceutically-acceptable salt thereof; or

(c) tert-butyl 2-{7-[4-(4-cyanophenyl)butyl]-9-oxa-3,7-diazabicyclo-[3.3.1]non-3-yl}ethylcarbamate: 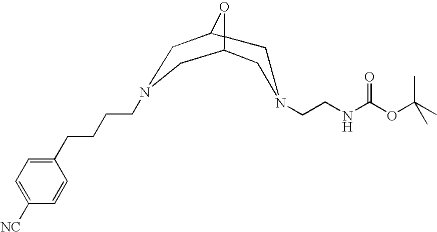
in the form of the free base, which compound is referred to hereinafter as Compound C or a pharmaceutically-acceptable salt thereof; or

(d) tert-butyl 2-{7-[(2S)-3-(4-cyanophenoxy)-2-hydroxypropyl]-9-oxa-3,7-diazabicyclo[3.3.1]non-3-yl}ethylcarbamate: 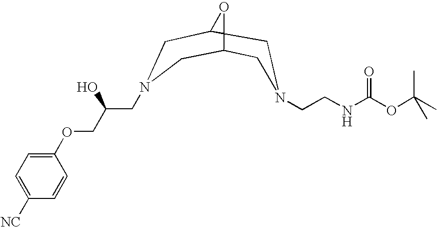
in the form of the free base, which compound is referred to hereinafter as Compound D or a pharmaceutically-acceptable salt thereof;

wherein each of components (1) and (2) is formulated in admixture with a pharmaceutically-acceptable adjuvant, diluent or carrier.

The combination product according to the invention provides for the administration of a compound of claim 1 in WO 02/44145 or a compound of claim 20 in WO 02/44145 in conjunction with (1) a compound as defined in claim 1 of WO 01/28992 or (2) a compound of claim 34 of WO 01/28992 or (3) Compound A or B or C or D (or pharmaceutically-acceptable salts thereof), and may thus be presented either as separate formulations, wherein at least one of those formulations comprises a compound of claim 1 in WO 02/44145 or a compound of claim 20 in WO 02/44145 and at least one comprises (1) a compound as defined in claim 1 of WO 01/28992 or (2) a compound of claim 34 of WO 01/28992 or

Compound A or B or C or D (or pharmaceutically-acceptable salts thereof), or may be presented (i.e. formulated) as a combined preparation (i.e. presented as a single formulation including a compound of claim 1 in WO 02/44145 or a compound of claim 20 in WO 02/44145 and (1) a compound as defined in claim 1 of WO 01/28992 or (2) a compound of claim 34 of WO 01/28992 or

(3) Compound A or B or C or D (or pharmaceutically-acceptable salts thereof)).

Thus, there is further provided:

(1) a pharmaceutical formulation including, a compound of claim 1 in WO 02/44145 or a compound of claim 20 in WO 02/44145 or a pharmaceutically-acceptable derivative thereof, and (1) a compound as defined in claim 1 of WO 01/28992 or (2) a compound of claim 34 of WO 01/28992 or (3) Compound A or B or C or D (or pharmaceutically-acceptable salts thereof), in admixture with a pharmaceutically-acceptable adjuvant, diluent or carrier (which formulation is hereinafter referred to as a “combined preparation”); and

(2) a kit of parts comprising components:

(a) a pharmaceutical formulation including a compound of claim 1 in WO 02/44145 or a compound of claim 20 in WO 02/44145 or a pharmaceutically-acceptable derivative thereof, in admixture with a pharmaceutically-acceptable adjuvant, diluent or carrier; and (b) a pharmaceutical formulation including (1) a compound as defined in claim 1 of WO 01/28992 or (2) a compound of claim 34 of WO 01/28992 or (3) Compound A or B or C or D (or pharmaceutically-acceptable salts thereof) in admixture with a pharmaceutically-acceptable adjuvant, diluent or carrier,

which components (a) and (b) are each provided in a form that is suitable for administration in conjunction with the other.

According to a further aspect of the invention, there is provided a method of making a kit of parts as defined above, which method comprises bringing a component (a), as defined above, into association with a component (b), as defined above, thus rendering the two components suitable for administration in conjunction with each other.

By bringing the two components “into association with” each other, we include that components (a) and (b) of the kit of parts may be:

(i) provided as separate formulations (i.e. independently of one another), which are subsequently brought together for use in conjunction with each other in combination therapy; or

(ii) packaged and presented together as separate components of a “combination pack” for use in conjunction with each other in combination therapy.

Thus, there is further provided a kit of parts comprising:

(I) one of components (a) and (b) as defined herein; together with

(II) instructions to use that component in conjunction with the other of the two components.

The kits of parts described herein may comprise more than one formulation including an appropriate quantity/dose of a compound of claim 1 in WO 02/44145 or a compound of claim 20 in WO 02/44145 or derivative thereof, and/or more than one formulation including an appropriate quantity/dose of (1) a compound as defined in claim 1 of WO 01/28992 or (2) a compound of claim 34 of WO 01/28992 or

(3) Compound A or B or C or D (or pharmaceutically-acceptable salts thereof), in order to provide for repeat dosing. If more than one formulation (comprising either active compound) is present, such formulations may be the same, or may be different in terms of the dose of a compound of claim 1 in WO 02/44145 or a compound of claim 20 in WO 02/44145 (or derivative) or (1) a compound as defined in claim 1 of WO 01/28992 or (2) a compound of claim 34 of WO 01/28992 or (3) Compound A or B or C or D (or pharmaceutically-acceptable salts thereof), chemical composition and/or physical form.

A further aspect of the invention provides a method of treatment of a condition where anticoagulant therapy is indicated, which comprises administration of a pharmaceutical formulation including a compound of claim 1 in WO 02/44145 or a compound of claim 20 in WO 02/44145 (or a pharmaceutically-acceptable derivative thereof), and (1) a compound as defined in claim 1 of WO 01/28992 or (2) a compound of claim 34 of WO 01/28992 or (3) Compound A or B or C or D (or pharmaceutically-acceptable salts thereof), in admixture with a pharmaceutically-acceptable adjuvant, diluent or carrier.

A further aspect of the invention provides a method of treatment of a condition where anticoagulant therapy is indicated (by which we mean where anticoagulation is required), which comprises administration of:

(a) a pharmaceutical formulation including a compound of claim 1 in WO 02/44145 or a compound of claim 20 in WO 02/44145 or a pharmaceutically-acceptable derivative thereof, in admixture with a pharmaceutically-acceptable adjuvant, diluent or carrier; in conjunction with

(b) a pharmaceutical formulation including (1) a compound as defined in claim 1 of WO 01/28992 or (2) a compound of claim 34 of WO 01/28992 or (3) Compound A or B or C or D (or pharmaceutically-acceptable salts thereof), in admixture with a pharmaceutically-acceptable adjuvant, diluent or carrier,

to a patient suffering from, or susceptible to, such a condition.

For the avoidance of doubt, as used herein, the term “treatment” includes therapeutic and/or prophylactic treatment.

With respect to the kits of parts as described herein, by “administration in conjunction with”, we include that respective formulations comprising a compound of claim 1 in WO 02/44145 or a compound of claim 20 in WO 02/44145 (or derivative thereof) and (1) a compound as defined in claim 1 of WO 01/28992 or (2) a compound of claim 34 of WO 01/28992 or (3) Compound A or B or C or D (or pharmaceutically-acceptable salts thereof), are administered, sequentially, separately and/or simultaneously, over the course of treatment of the relevant condition, which condition may be acute or chronic.

Thus, in respect of the combination product according to the invention, the term “administration in conjunction with” includes that the two components of the combination product (a compound of claim 1 in WO 02/44145 or a compound of claim 20 in WO 02/44145 and (1) a compound as defined in claim 1 of WO 01128992 or (2) a compound of claim 34 of WO 01/28992 or (3) Compound A or B or C or D (or pharmaceutically-acceptable salts thereof)) are administered (optionally repeatedly), either (in the case of a combined preparation) together, or (in the case of a kit of parts) sufficiently closely in time, to enable a beneficial effect for the patient, that is greater, over the course of the treatment of the relevant condition, than if either a formulation comprising a compound of claim 1 in WO 02144145 or a compound of claim 20 in WO 02/44145, or a formulation comprising (1) a compound as defined in claim 1 of WO 01/28992 or (2) a compound of claim 34 of WO 01/28992 or

(3) Compound A or B or C or D (or pharmaceutically-acceptable salts thereof), are administered (optionally repeatedly) alone, in the absence of the other component, over the same course of treatment. Determination of whether a combination provides a greater beneficial effect in respect of, and over the course of treatment of, a particular condition, will depend upon the condition to be treated or prevented, but may be achieved routinely by the skilled person.

Further, in the context of a kit of parts according to the invention, the term “in conjunction with” includes that one or other of the two formulations may be administered (optionally repeatedly) prior to, after, and/or at the same time as, administration with the other component. When used in this context, the terms “administered simultaneously” and “administered at the same time as” include that individual doses of a compound of claim 1 in WO 02/44145 or a compound of claim 20 in WO 02/44145 (or derivative thereof) and (1) a compound as defined in claim 1 of WO 01/28992 or (2) a compound of claim 34 of WO 01/28992 or (3) Compound A or B or C or D (or pharmaceutically-acceptable salts thereof), are administered within 48 hours (e.g. 24 hours) of each other.

Suitable daily doses of the compounds of a compound of claim 1 in WO 02/44145 or a compound of claim 20 in WO 02/44145 (or derivative thereof) in therapeutic treatment of humans are about 0.001-100 mg/kg body weight at peroral administration and 0.001-50 mg/kg body weight at parenteral administration.

Suitable doses of (1) a compound as defined in claim 1 of WO 01/28992 or (2) a compound of claim 34 of WO 01/28992 or (3) Compound A or B or C or D (or pharmaceutically-acceptable salts thereof), in the therapeutic and/or prophylactic treatment of mammalian, especially human, patients may be determined routinely by the medical practitioner or other skilled person, and include the respective doses discussed in WO 01/28992 which is hereby incorporated by reference.

In the case of antiarrhythmic oxabispidines typical daily doses of (1) a compound as defined in claim 1 of WO 01/28992 or (2) a compound of claim 34 of WO 01/28992 or (3) Compound A or B or C or D (or pharmaceutically-acceptable salts thereof), are in the range 10 to 2000 mg, e.g. 25, such as 30, to 1200 mg of free base (i.e., in the case of a salt, excluding any weight resulting from the presence of a counter ion), irrespective of the number of compositions (e.g. tablets) that are administered during the course of that day. Preferred daily doses are in the range 50 to 1000 mg, such as 100 to 500 mg, for example 150 mg, 200 mg, 250 mg, 300 mg, 350 mg, 400 mg or 450 mg. Typical doses in individual compositions of the invention (e.g. tablets) are thus in the range 15 to 500 mg, for example 40 to 400 mg eg for example 150 mg, 200 mg, 250 mg, 300 mg, 350 mg or 400 mg.

Specifically claimed herein are specific fixed dose combinations where any dose stated for a compound of claim 1 in WO 02/44145 or a compound of claim 20 in WO 02/44145 (or derivative thereof) is combined with any dose stated for the antiarrhythmic oxabispidine, including the doses stated as limits for the ranges described.

In any event, the physician, or the skilled person, will be able to determine the actual dosage which will be most suitable for an individual patient, which is likely to vary with the condition that is to be treated, as well as the age, weight, sex and response of the particular patient to be treated. The above-mentioned dosages are exemplary of the average case; there can, of course, be individual instances where higher or lower dosage ranges are merited, and such are within the scope of this invention.

When separate formulations are administered, the sequence in which the formulations comprising a compound of claim 1 in WO 02/44145 or a compound of claim 20 in WO 02/44145 (or derivative thereof), and the antiarrhythmic oxabispidine (or derivative thereof), may be administered (i.e. whether, and at what point, sequential, separate and/or simultaneous administration takes place) may be determined by the physician or skilled person. For example, the sequence may depend upon many factors that will be evident to the skilled person, such as whether, at any time during the course or period of treatment, one or other of the formulations cannot be administered to the patient for practical reasons (e.g. the patient is unconscious and thus unable to take an oral formulation comprising either a compound of claim 1 in WO 02/44145 or a compound of claim 20 in WO 02/44145 (or derivative thereof) or the antiarrhythmic oxabispidine).

The method described herein may have the advantage that, in the treatment of conditions where anticoagulant therapy is indicated, it may be more convenient for the physician and/or patient than, be more efficacious than, be less toxic than, have a broader range of activity than, be more potent than, produce fewer side effects than, or that it may have other useful pharmacological properties over, similar methods known in the prior art for the treatment of such conditions.

A compound of claim 1 in WO 02/44145 or a compound of claim 20 in WO 02/44145 (or derivative thereof) and derivatives thereof, may be administered for systemic delivery using appropriate means of administration that are known to the skilled person.

Thus, in accordance with the invention, a compound of claim 1 in WO 02/44145 or a compound of claim 20 in WO 02/44145 (or derivative thereof) and derivatives thereof, may be administered orally, intravenously, subcutaneously, buccally, rectally, dermally, nasally, tracheally, bronchially, topically, by any other parenteral route, or via inhalation, in the form of a pharmaceutical preparation comprising the active ingredient in a pharmaceutically-acceptable dosage form. Depending on the disorder, and the patient, to be treated, as well as the route of administration, the compositions may be administered at varying doses.

Preferred modes of delivery are systemic. For a compound of claim 1 in WO 02/44145 or a compound of claim 20 in WO 02/44145 (or derivative thereof), preferred modes of lo administration are oral, parenteral, more preferably intravenous, and especially subcutaneous. Preferred modes of administration are oral.

In the therapeutic treatment of mammals, and especially humans, a compound of claim 1 in WO 02/44145 or a compound of claim 20 in WO 02/44145 (or derivative thereof) may be administered alone, but will generally be administered as a pharmaceutical formulation in admixture with a pharmaceutically-acceptable adjuvant, diluent or carrier, which may be selected with due regard to the intended route of administration and standard pharmaceutical practice.

The preparation of suitable formulations may be achieved non-inventively by the skilled person using routine techniques.

The combinations of the present invention are useful in both the prophylaxis and the treatment of cardiac arrhythmias, in particular atrial and ventricular arrhythmias (such as atrial fibrillation (e.g. atrial flutter)) and NVAF.

The combinations of the invention are thus indicated in the treatment or prophylaxis of cardiac diseases, or in indications related to cardiac diseases, in which arrhythmias are believed to play a major role, including ischemic heart disorders, sudden heart attack, myocardial infarction, heart failure, cardiac surgery and thromboembolic events.

The term “ischemic disorders” will be understood by those skilled in the art to include any condition, the results of which include a restriction in blood flow in a part of the body. In this context, the term will also be understood to include thrombosis and hypercoagulability in blood and/or organs, tissues, etc.

The term “thrombosis” will be understood by those skilled in the art to include the formation, development or presence of a thrombus in animals including man, and which may result in embolism and/or ischemia. The term may thus include conditions such as atrophic thrombosis, arterial thrombosis, cardiac thrombosis, coronary thrombosis, creeping thrombosis, infective thrombosis, mesenteric thrombosis, placental thrombosis, propagating thrombosis, traumatic thrombosis and venous thrombosis.

The term “hypercoagulability” includes any state in which the blood is more readily coagulated than usual.

The term “NVAF” may be understood by those skilled in the art to mean grossly disorganised atrial electrical activity, which is irregular in respect of both rate and rhythm, leading to a hypercoagulable state and an increased risk of thrombosis originating from the left heart chambers, and particularly the left atrium. The term may thus also be understood to include AF (chronic, persistent, permanent and/or intermittent (paroxysmal)) in the absence of heart valvular disease (mostly rheumatic heart valvular disease e.g. mitral stenosis), or prosthesis, and to exclude patients with rheumatic mitral stenosis.

Particular disease states that may be mentioned include the prevention/treatment of ischemic heart disease, myocardial infarction, systemic embolic events in e.g. the kidneys, spleen etc, and, more particularly, of cerebral ischemia, including cerebral thrombosis, cerebral embolism and/or cerebral ischemia associated with non-cerebral thrombosis or embolism (in other words, the treatment/prophylaxis of thrombotic, or ischemic, stroke and of transient ischemic attack (TIA)) in patients with, or at risk of, NVAF. The skilled person will appreciate that patients with NVAF who are at risk of stroke include elderly patients generally (e.g. those with an age of greater than 75 years); patients with complicating health factors, such as hypertension, left ventricular dysfunction (e.g. left ventricular ejection fraction (LVEF) of less than 40%), symptomatic congestive heart failure, diabetes mellitus (especially in those patients of 65 years of age or greater) and/or coronary heart or artery disease (especially in those patients of 65 years of age or greater); and/or patients with a history of stroke, TIA and/or systemic embolism, all of which factors may predispose such patients to stroke and/or thromboembolic events.

According to a further aspect of the invention, there is provided a method of treatment of an arrhythmia which method comprises administration of a combination of the invention to a person suffering from, or susceptible to, such a condition.

According to a further aspect of the invention, there is provided a method of treatment of atrial fibrillation which method comprises administration of a combination of the invention to a person suffering from, or susceptible to, such a condition.

According to a further aspect of the invention, there is provided a method of treatment of atrial flutter which method comprises administration of a combination of the invention to a person suffering from, or susceptible to, such a condition.

For the avoidance of doubt, by “treatment” we include the therapeutic treatment, as well as the prophylaxis, of a condition.

It is expected that the combinations of the present invention may provide one or more of the following advantages. Synergy between the components in terms of:
response rate
patient survival rate
time to disease progression
dose/response effects leading to lower doses with same efficacy.

Alternatively, it is expected that the combinations of the present invention may provide one or more of the following advantages:

lower toxicity/reduced side effects with similar/improved efficacy;

improved physical properties, e.g. storage stability, flow properties etc.;

ease of formulation for example, reduced drug/drug incompatibility problems;

reduced drug/ drug interaction problems on administration, for example possible changes in metabolism of one drug caused by the effect of the other drug;

improved patient compliance;

improved quality of life;

convenient dosing regimes; or

lack of diminishing effects of one drug caused by the presence of the other drug.

It is expected that the combination of the present invention will lead to a reduced incidence of strokes in patients suspectible to strokes by the treatment and prevention of atrial fibrillation.

Improved patient compliance may be demonstrated by methods known to those skilled in the art, for example by supplying patients with blister packs containing the combination of the present invention wherein the date and time of the removal of a drug from the blister pack is recorded.

In a further aspect the present invention provides a process for the preparation of a combination product as described earlier comprising formulating (1) a compound of claim 1 in WO 02/44145 or a compound of claim 20 in WO 02/44145 (or derivative thereof) with a pharmaceutically acceptable diluent or carrier; and then formulating (1) a compound as defined in claim 1 of a compound of claim 1 in WO 02/44145 or a compound of claim 20 in WO 02/44145 (or derivative thereof) or (2) a compound of claim 34 of WO 01/28992 or (3) Compound A or B or C or D (or pharmaceutically-acceptable salts thereof) in a dose as previously described herein with a pharmaceutically acceptable diluent or carrier; and then combining these formulations to provide a combination product as previously described herein.

The combination product of the present invention can be used both in conversion of AF into normal sinus rhytm and maintenance of said sinus rhytm.

The combination product of the present invention can be used to treat both symptomatic and asymptomatic atrial fibrillation.

The combination product of the present invention can be used to treat paroxysmal AF, persistent AF and permanent AF.

The ratios of the active compound in the combination product of the present invention can be in the range of 100:1, 50:1, 20:1, 10:1, 5:1, 2:1, 1:1, 1:2, 1:5, 1:10, 1:50 or 1:100.

The present invention therefore provides the additional advantage that it allows tailoring of treatment to the needs of a particular patient population. Examples of such particular patient population are; 1) elderly patient, especially over the age of 60, preferably over the age of 70, more preferably over the age of 80; 2) female patients; 3) patients suffering from any of the following conditions; hypertension, heart failure, and diabetes.

The combination product of the present invention, is either additive or synergistic in effect in the treatment of AF, in particular paroxysmal AF, persistent AF and permanent AF of a particular patient population. Examples of such particular patient population are; 1) elderly patient, especially over the age of 60, preferably over the age of 70, more preferably over the age of 80; 2) female patients; 3) patients suffering from any of the following conditions; hypertension, heart failure, and diabetes.

Compounds of WO 01/28992 may be prepared as described therein.

Compounds of WO 02144145 may be prepared as described below and analogous methods thereof.

General Experimental Details

TLC was performed on silica gel. Chiral HPLC analysis was performed using a 46 mm×250 mm Chiralcel OD column with a 5 cm guard column. The column temperature was maintained at 35° C. A flow rate of 1.0 mL/min was used. A Gilson 115 UV detector at 228 nm was used. The mobile phase consisted of hexanes, ethanol and trifluroacetic acid and the appropriate ratios are listed for each compound. Typically, the product was dissolved in a minimal amount of ethanol and this was diluted with the mobile phase.

LC-MS/MS was performed using a HP-1100 instrument equipped with a CTC-PAL injector and a 5 μm, 4×100 mm ThermoQuest, Hypersil BDS-C18 column. An API-3000 (Sciex) MS detector was used. The flow rate was 1.2 mL/min and the mobile phase (gradient) consisted of 10-90% acetonitrile with 90-10% of 4 mM aq. ammonium acetate, both containing 0.2% formic acid.

¹H NMR spectra were recorded using tetramethylsilane as the internal standard. ¹³C NMR spectra were recorded using the listed deuterated solvents as the internal standard.

### EXAMPLE 1

### Ph(3-Cl)(5-OCHF₂)—(R)CH(OH)C(O)-Aze-Pab(OcBu)

### (i) 3-Chloro-5-methoxybenzaldehyde

3,5-Dichloroanisole (74.0 g, 419 mmol) in THF (200 mL) was added dropwise to magnesium metal (14.2 g, 585 mmol, pre-washed with 0.5 N HCl) in THF (100 mL) at 25° C. After the addition, 1,2-dibromoethane (3.9 g, 20.8 mmol) was added dropwise. The resultant dark brown mixture was heated at reflux for 3 h. The mixture was cooled to 0° C., and N,N-dimethylformamide (60 mL) was added in one portion. The mixture was partitioned with diethyl ether (3×400 mL) and 6N HCl (500 mL). The combined organic extracts were washed with brine (300 mL), dried (Na₂SO₄), filtered and concentrated in vacuo to give an oil. Flash chromatography (2×) on silica gel eluting with Hex:EtOAc (4:1) afforded the sub-title compound (38.9 g, 54%) as a yellow oil.

¹H NMR (300 MHz, CDCl₃) Λ 9.90 (s, 1H), 7.53 (s, 1H), 7.38 (s, 1H), 7.15 (s, 1H), 3.87 (s, 3H).

### (ii) 3-Chloro-5-hydroxybenzaldehyde

A solution of 3-chloro-5-methoxybenzaldehyde (22.8 g, 134 mmol; see step (i) above) in CH₂Cl₂ (250 mL) was cooled to 0° C. Boron tribromide (15.8 mL, 167 mmol) was added dropwise over 15 min. After stirring, the reaction mixture for 2 h, H₂O (50 mL) was added slowly. The solution was then extracted with Et₂O (2×100 mL). The organic layers were combined, dried (Na2SO₄), filtered and concentrated in vacuo. Flash chromatography on silica gel eluting with Hex:EtOAc (4:1) afforded the sub-title compound (5.2 g, 25%).

¹H NMR (300 MHz, CDCl₃) δ 9.85 (s, 1H), 7.35 (s, 1H), 7.20 (s, 1H), 7.10 (s,1H), 3.68 (s, 1H)

### (iii) 3-Chloro-5-difluoromethoxybenzaldehyde

A solution of 3-chloro-5-hydroxybenzaldehyde (7.5 g, 48 mmol; see step (ii) above) in 2-propanol (250 mL) and 30% KOH (100 mL) was heated to reflux. While stirring, CHClF₂ was bubbled into the reaction mixture for 2 h. The reaction mixture was cooled, acidified with 1N HCl and extracted with EtOAc (2×100 mL). The organics were washed with brine (100 mL), dried (Na₂SO₄), filtered and concentrated in vacuo. Flash chromatography on silica gel eluting with Hex:EtOAc (4:1) afforded the sub-title compound (4.6 g, 46%).

¹H NMR (300 MHz, CDCl₃) δ 9.95 (s, 1H), 7.72 (s, 1H), 7.52 (s, 1H), 7.40 (s, 1H), 6.60 (t, J_{H-F}=71.1 Hz, 1H)

### (iv) Ph(3-Cl)(5-OCHF₂)—(R,S)CH(OTMS)CN

A solution of 3-chloro-5-difluoromethoxybenzaldehyde (4.6 g, 22.3 mmol; see step (iii) above) in CH₂Cl₂ (200 mL) was cooled to 0° C. ZnI₂ (1.8 g, 5.6 mmol) and trimethylsilyl cyanide (2.8 g, 27.9 mmol) were added and the reaction mixture was allowed to warm to room temperature and stirred for 15 h. The mixture was partially concentrated in vacuo yielding the sub-title compound as a liquid, which was used directly in step (v) below without further purification or characterization.

### (v) Ph(3-Cl)(5-OCHF₂)—(R,S)CH(OH)C(NH)OEt

Ph(3-Cl)(5-OCHF₂)—(R,S)CH(OTMS)CN (6.82 g, assume 22.3 mmol; see step (iv) above) was added dropwise to HCl/EtOH (500 mL). The reaction mixture was stirred 15 h, then partially concentrated in vacuo yielding the sub-title compound as a liquid, which was used in step (vi) without further purification or characterization.

### (vi) Ph(3-Cl)(5-OCHF₂)—(R,S)CH(OH)C(O)OEt

Ph(3-Cl)(5-OCHF₂)-(R,S)CH(OH)C(NH)OEt (6.24 g, assume 22.3 mmol; see step (v) above) was dissolved in THF (250 ML), 0.5M H₂SO₄ (400 mL) was added and the reaction was stirred at 40° C. for 65 h, cooled and then partially concentrated in vacuo to remove most of the THF. The reaction mixture was then extracted with Et₂O (3×100 mL), dried (Na₂SO₄), filtered and concentrated in vacuo to afford the sub-title compound as a solid, which was used in step (vii) without further purification or characterization.

### (vii) Ph(3-Cl)(5-OCHF₂)—(R,S)CH(OH)C(O)OH

A solution of Ph(3-Cl)(5-OCHF₂)—(R,S)CH(OH)C(O)OEt (6.25 g, assume 22.3 mmol; see step (vi) above) in 2-propanol (175 mL) and 20% KOH (350 mL) was stirred at room temperature 15 h. The reaction was then partially concentrated in vacuo to remove most of the 2-propanol. The remaining mixture was acidified with 1M H₂SO₄, extracted with Et₂O (3×100 mL), dried (Na₂SO₄) and concentrated in vacuo to give a solid. Flash chromatography on silica gel eluting with CHCl₃:MeOH:concentrated NH₄OH (6:3:1) afforded the ammonium salt of the sub-title compound. The ammonium salt was then dissolved in a mixture of EtOAc (75 mL) and H₂O (75 mL) and acidified with 2N HCl.

The organic layer was separated and washed with brine (50 mL), dried (Na₂SO₄) and concentrated in vacuo to afford the sub-title compound (3.2 g, 57% from steps (iv) to (vii)).

¹H NMR (300 MHz, CD₃OD) δ 7.38 (s, 1H), 7.22 (s, 1H), 7.15 (s, 1H), 6.89 (t, J_{H-F}=71.1 Hz, 1H), 5.16 (s, 1H)

### (viii) Ph(3-Cl)(5-OCHF₂)—(R)CH(OH)C(O)OH (a) and Ph(3-Cl)(5-OCHF₂)-(S)CH(OAc)C(O)OH (b)

A mixture of Ph(3-Cl)(5-OCHF₂)—(R,S)CH(OH)C(O)OH (3.2 g, 12.7 mmol; see step (vii) above) and Lipase PS “Amano” (˜2.0 g) in vinyl acetate (125 mL) and MTBE (125 mL) was heated at reflux for 48 h. The reaction mixture was cooled, filtered through Celite® and the filter cake washed with EtOAc. The filtrate was concentrated in vacuo and subjected to flash chromatography on silica gel eluting with CHCl₃:MeOH:concentrated NH₄OH (6:3:1) yielding the ammonium salts of the sub-title compounds (a) and (b). Compound (a) as a salt was dissolved in H₂O, acidified with 2N HCl and extracted with EtOAc. The organic layer was washed with brine, dried (Na₂SO₄), filtered and concentrated in vacuo to afford the sub-title compound (a) (1.2 g, 37%).

For sub-title compound (a)

hu 1H NMR (300 MHz, CD₃OD) δ 7.38 (s, 1H), 7.22 (s, 1H), 7.15 (s, 1H), 6.89 (t, J_{H-F}=71.1 Hz, 1H), 5.17 (s, 1H)

### (ix) Ph(3-Cl)(5-OCHF₂)—(R)CH(OH)C(O)-Aze-Pab(Teoc)

To a solution of Ph(3-Cl)(5-OCHF₂)—(R)CH(OH)C(O)OH (1.1 g, 4.4 mmol; see step (viii) above) and H-Aze-Pab(Teoc) (see international patent application WO 00/42059, 2.6 g, 5.7 mmol) in DMF (50 mL) at 0° C. was added PyBOP (2.8 g, 5.3 mmol) and collidine (1.3 g, 10.6 mmol). The reaction was stirred at 0° C. for 2 h and then at room temperature for an additional 15 h. The reaction mixture was concentrated in vacuo and flash chromatographed on silica gel (3×), eluting first with CHCl₃:EtOH (9:1), then with EtOAc:EtOH (20:1) and finally eluting with CH₂Cl₂:CH₃OH (95:5) to afford the sub-title compound (1.0 g, 37%) as a white solid.

¹H NMR (300 MHz, CD₃OD, mixture of rotamers) δ 7.79-7.85 (d, J =8.7 Hz, 2H), 7.15-7.48 (m, 5H), 6.89 and 6.91 (t, J_{H-F}=71.1 Hz, 1H), 5.12 and 5.20 (s, 1H), 4.75-4.85 (m, 1H), 3.97-4.55 (m, 6H), 2.10-2.75 (m, 2H), 1.05-1.15 (m, 2H), 0.09 (s, 9H)

MS (m/z) 611 (M+1)⁺

### (x) Ph(3-Cl)(5-OCHF₂)—(R)CH(OH)C(O)-Aze-Pab(OcBu, Teoc)

Ph(3-Cl)(5-OCHF₂)—(R)CH(OH)C(O)-Aze-Pab(Teoc) (0.051 g, 0.08 mmol; see step (ix) above), was dissolved in 3 mL of acetonitrile and 0.062 g (0.5 mmol) of O-cyclobutylhydroxylamine hydrochloride was added. The mixture was heated at 70° C. for 4.5 h. The solvent was evaporated and the residue was partitioned between water and ethyl acetate. The aqueous phase was extracted two more times with ethyl acetate and the combined organic phase was washed with water, brine, dried (Na₂SO₄), filtered and evaporated. Yield: 0.054 g (95%).

¹H-NMR (400 MHz; CD₃OD): δ 8.66-8.50 (m, 1H), 7.45 (d, 2H), 7.29 (m, 3H), 7.15 (m, 2H), 6.88 (t, 1H major rotamer), 6.85 (t, 1H minor rotamer), 5.18 (s, 1H major rotamer), 5.12 (s, 1H minor rotamer), 5.16 (m, 1H minor rotamer), 4.78 (m, 1H major rotamer), 4.70 (m, 1H), 4.50-4.30 (m, 3H), 4.19-3.93 (m, 3H), 2.71-2.44 (m, 1H), 2.34-2.11 (m, 5H), 1.78 (m, 1H), 1.62 (m, 1H), 0.96 (m, 2H), 0.01 (s, 9H)

### (xi) Ph(3-Cl)(5-OCHF₂)—(R)CH(OH)C(O)-Aze-Pab(OcBu)

Ph(3-Cl)(5-OCHF₂)—(R)CH(OH)C(O)-Aze-Pab(OcBu, Teoc) (0.054 g, 0.08 mmol; see step (x) above), was dissolved in 0.5 mL of CH₂Cl₂ and 3 mL of TFA. The reaction was allowed to proceed for 60 minutes. TFA was evaporated and the residue was purified using preparative HPLC. The fractions of interest were pooled and freeze-dried (2×), yielding 23 mg (54%) of the title compound.

MS (m/z) 536 (M−1)⁻; 538 (M+1)⁺

¹H-NMR (400 MHz; CD₃OD): δ 7.56 (d, 2H), 7.33 (m, 3H), 7.15 (m, 2H), 6.89 (t, 1H major rotamer), 6.86 (t, 1H minor rotamer), 5.18 (s, 1H major rotamer; and m, 1H minor rotamer), 5.11 (s, 1H minor rotamer), 4.77 (m, 1H major rotamer), 4.58 (m, 1H), 4.42 (m, 2H), 4.34 (m, 1H major rotamer), 4.15 (m, 1H major rotamer), 4.06 (m. 1H minor rotamer), 3.97 (m, 1H minor rotamer), 2.66 (m, 1H minor rotamer), 2.52 (m, 1H major rotamer), 2.33-2.25 (m, 3H), 2.01-2.20 (m, 2H), 1.75 (m, 1H), 1.59 (m, 1H)

¹³C-NMR (100 MHz; CD₃OD) (carbonyl and/or amidine carbons, rotamers) δ 172.4, 172.3, 171.9, 171.4, 152.3

### EXAMPLE 2

### Ph(3-Cl)(5-OCHF₂)—(R)CH(OH)C(O)-Aze-Pab(OH)

### (i) Ph(3-Cl)(5-OCHF₂)—(R)CH(OH)C(O)-Aze-Pab(OH, Teoc)

Ph(3-Cl)(5-OCHF₂)—(R)CH(OH)C(O)-Aze-Pab(Teoc) (0.148 g, 0.24 mmol; see Example 1(ix) above), was dissolved in 9 mL of acetonitrile and 0.101 g (1.45 mmol) of hydroxylamine hydrochloride was added. The mixture was heated at 70° C. for 2.5 h, filtered through Celite® and evaporated. The crude product (0.145 g; 75% pure) was used directly in the next step without further purification.

### (ii) Ph(3-Cl)(5-OCHF₂)—(R)CH(OH)C(O)-Aze-Pab(OH)

Ph(3-Cl)(5-OCHF₂)—(R)CH(OH)C(O)-Aze-Pab(OH, Teoc) (0.145 g, 0.23 mmol; see step (i) above), was dissolved in 0.5 mL of CH₂Cl₂ and 9 mL of TFA. The reaction was allowed to proceed for 60 minutes. TFA was evaporated and the residue was purified using preparative HPLC. The fractions of interest were pooled and freeze-dried (2×), yielding 72 mg (yield over two steps 62%) of the title compound.

MS (m/z) 482 (M−1)⁻; 484 (M+1)⁺

¹H-NMR (400 MHz; CD₃OD): δ 7.58 (d, 2H), 7.33 (m, 3H), 7.15 (m, 2H), 6.89 (t, 1H major rotamer), 6.86 (t, 1H minor rotamer), 5.18 (s, 1H major rotamer; and m, 1H minor rotamer), 5.12 (s, 1H minor rotamer), 4.77 (m, 1H major rotamer),4.42 (m, 2H), 4.34 (m, 1H major rotamer), 4.14 (m, 1H major rotamer), 4.06 (m, 1H minor rotamer), 3.95 (m, 1H minor rotamer), 2.66 (m, 1H minor rotamer), 2.50 (m, 1H major rotamer), 2.27 (m, 1H major rotamer), 2.14 (m, 1H minor rotamer)

¹³C-NMR (100 MHz; CD₃OD): (carbonyl and/or amidine carbons, rotamers) δ 172.4, 172.3, 172.0, 171.4 152.3, 152.1

### EXAMPLE 3

### Ph(3-Cl)(5-OCHF₂)—(R)CH(OH)C(O)-Aze-Pab

Ph(3-Cl)(5-OCHF₂)—(R)CH(OH)C(O)-Aze-Pab(Teoc) (0.045 g, 0.074 mmol; see Example 1(ix) above), was dissolved in 3 mL of TFA and allowed to react for 1 h. TFA was evaporated and the residue was freeze dried from water/acetonitrile to yield 0.043 g (100%) of the sub-title compound as its TFA salt.

¹H-NMR (400 MHz; CD₃OD) rotamers: δ 7.8-7.75 (m, 2H), 7.55-7.5 (m, 2H), 7.35 (m, 1H, major rotamer), 7.31 (m, 1H, minor rotamer), 7.19 (m, 1H, major rotamer), 7.15 (m, 1H), 7.12 (m, 1H, minor rotamer), 6.89 (t, 1H, major rotamer), 6.87 (t, 1H, minor rotamer), 5.22 (m, 1H, minor rotamer), 5.20 (s, 1H, major rotamer), 5.13 (s, 1H, minor rotamer), 4.80 (m, 1H, major rotamer), 4.6-4.4 (m, 2H), 4.37 (m, 1H, major rotamer), 4.19 (m, 1H, major rotamer), 4.07 (m, 1H, minor rotamer), 3.98 (m, 1H, minor rotamer), 2.70 (m, 1H, minor rotamer), 2.55 (m, 1H, major rotamer), 2.29 (m, 1H, major rotamer), 2.15 (m, 1H, minor rotamer)

¹³C-NMR (100 MHz; CD₃OD): (carbonyl and/or amidine carbons, rotamers) δ 172.6, 172.5, 172.0, 171.7, 167.0

MS (m/z) 465 (M−1)⁻, 467 (M+1)⁺

### EXAMPLE 4

### Ph(3-Cl)(5-OCHF₂)—(R)CH(OH)C(O)-Aze-Pab(COOcPentyl)

To a solution of Ph(3-Cl)(5-OCHF₂)—(R)CH(OH)C(O)-Aze-Pab×TFA (74 mg, 0.13 mmol; see Example 3 above) and cyclopentylchloroformate (44 mg, 0.30 mmol) in methylene chloride (5 mL) was added aq. NaOH (0.5 mL, 2M, 1 mmol). The mixture was stirred at room temperature and the reaction was monitored with HPLC. After 2.5 hours, water was added and the liquid phases were separated. The aqueous phase was extracted twice with methylene chloride. The combined organic phases were dried (MgSO₄) and purified on silica gel (first methylene chloride, then EtOAc).

After removal of the solvents in vacuo, the solid residue was dissolved in water/acetonitrile and freeze-dried to afford the title compound as a white solid. Yield: 33 mg (44%)

MS (m/z) 579 (M+1)⁺

¹H NMR (400 MHz; CD₃OD): Λ 7.79(d, 2H), 7.43-7.30(m, 5H), 7.20-7.11(m, 2H), 6.90(t, 1H, major rotamer), 6.87(t, 1H, minor rotamer), 5.19(dd, 1H, minor rotamer), 5.18(s, 1H, major rotamer), 5.13(m, 1H), 5.11(s, 1H, minor rotamer), 4.78(dd, 1H, major rotamer), 4.45(m, 2H), 4.35(m, 1H, major rotamer), 4.16(s, 1H, major rotamer), 4.06(s, 1H, minor rotamer), 3.97(s, 1H, minor rotamer), 2.68(m, 1H, minor rotamer), 2.52(s, 1H, major to rotamer), 2.28(s, 1H, major rotamer), 2.16(s, 1H, minor rotamer), 1.90(m, 2H), 1.77(m, 4H), 1.61(m, 2H)

¹³C NMR (carbonyl and/or amidine protons; 100 MHz): Λ 173.6, 173.1, 172.6, 170.3, 165.6

### EXAMPLE 5

### Ph(3-Cl)(5-OCHF₂)—(R)CH(OH)C(O)-Aze-Pab(Z)

The title compound was prepared according to the procedure described in Example 4 above starting from Ph(3-Cl)(5-OCHF₂)—(R)CH(OH)C(O)-Aze-Pab×TFA (73 mg, 0.13 mmol; see Example 3 above) and benzylchloroformate (35 mg, 0.21 mmol). Additional purification by reverse-phase HPLC (0.1M ammonium acetate/MeCN 40/60) was necessary. The appropriate fractions were concentrated in vacuo and extracted with EtOAc. Yield: 24 mg (32%).

MS (m/z) 602 (M+1)⁺

¹H NMR (400 MHz; CD₃OD): Λ 7.80(d, 2H), 7.43-7.25(m, 8H), 7.20-7.10(m, 2H), 6.90(t, 1H, major rotamer), 6.88(t, 1H, minor rotamer), 5.18(dd, 1H, minor rotamer), 5.18(s, 2H), 5.17(s, 1H, rotamer), 5.11(s, 1H, rotamer), 4.78(dd, 1H, major rotamer), 4.45(m, 2H), 4.34(m, 1H, major rotamer), 4.15(s, 1H, major rotamer), 4.06(s, 1H, minor rotamer), 3.97(s, 1H, minor rotamer), 2.66(m, 1H, minor rotamer), 2.51(s, 1H, major rotamer), 2.27(s, 1H, major rotamer), 2.15(s, 1H, minor rotamer)

¹³C NMR (carbonyl and/or amidine protons; 100 MHz): Λ 173.6, 173.1, 172.6, 170.5, 164.9

### EXAMPLE 6

### Ph(3-Cl)(5-OCF₃)—(R)CH(OH)C(O)-Aze-Pab×TFA

### (i) 2-Nitro-5-trifluoromethoxybenzoic acid

To a solution of 3-trifluoromethoxybenzoic acid (49.0 g, 0.24 mol) in sulfuric acid (500 mL) at less than 0° C. (ice-MeOH bath) was added a solution of potassium nitrate (31.3 g, 0.31 mol) in sulfuric acid (200 mL) over 20 minutes. The resulting solution was stirred at 0° C. for 2 hours, then warmed to room temperature and stirred for 18 hours. The reaction was poured into ice and the resulting acidic solution was extracted with EtOAc (5×). The combined organics were washed with H₂O (1×), brine (2'3), H₂O (1×) and brine (1×), dried (Na₂SO₄), filtered and concentrated in vacuo to give the crude sub-title compound (65.7 g) as a solid contaminated with HOAc. The crude sub-title compound was dissolved in EtOAc and toluene and concentrated in vacuo to give a HOAc free solid (58.4 g, 97%) that was used in the next step without further purification.

¹H NMR (300 MHz, CDCl₃): Λ 10.10 (br s, 1H), 8.02 (d, 1H, J=8 Hz), 7.69 (d, 1H, J=2 Hz), 7.54 (dd, 1H, J=2 Hz, J=8 Hz)

### (ii) 2-Amino-5-trifluoromethoxybenzoic acid

To a solution of 2-nitro-5-trifluoromethoxybenzoic acid (56.8 g, 0.23 mol; see step (i) above) in EtOH (1000 mL) was added 10% Pd/C (5.7 g). The resulting solution was flushed with H₂ for 5 h, filtered through Celite® and concentrated in vacuo to give the crude sub-title compound (49.7 g, 98%) as a solid that was used in the next step without further purification.

¹H NMR (300 MHz, CD₃OD): Λ 7.66 (m, 1H), 7.17 (d, 1H, J=8 Hz), 6.77 (d, 1H, J=8 Hz)

### (iii) 2-Amino-3-chloro-5-trifluoromethoxybenzoic acid

To a solution of 2-amino-5-trifluoromethoxybenzoic acid (49.0 g, 0.22 mol; see step (ii) above) in HOAc (1200 mL) was slowly added sulfuryl chloride (41.8 g, 0.31 mol). Gas evolution was observed. The resulting heterogeneous mixture was stirred at room temperature for 1 h. Additional HOAc (300 mL) was added to aid stirring, followed by sulfuryl chloride in 5 mL portions until the starting material was consumed based on TLC analysis. The reaction was concentrated in vacuo to give solids that were flushed on a rotary evaporator with EtOAc (2×) followed by Et₂O (1×) to remove the HOAc. The resulting solids were further dried to give the HCl salt of the crude sub-title compound (60.5 g, 94%), which was used in the next step without further purification.

¹H NMR (300 MHz, CD₃OD): Λ 7.72 (s, 1H), 7.44 (s, 1H), 7.22 (s, exchangeables)

### (iv) 3-Chloro-5-trifluoromethoxybenzoic acid

To a solution of 2-amino-3-chloro-5-trifluoromethoxybenzoic acid (60.5 g, assume 0.22 mol; see step (iii) above) in 1,4-dioxane (1000 mL) was added 6N HCl (750 mL). Some organics oiled out of solution. The dioxane solution was cooled to less than 0° C. (ice-MeOH bath). A solution of sodium nitrite (18.2 g, 0.26 mol) in H₂O (250 mL) was added over 15 minutes via an addition funnel. The resulting solution was stirred for 45 min. Hypophosphorous acid (221.5 mL of 50 wt % in H₂O, 291.2 g, 2.20 mol) was added slowly via an addition funnel. The solution was stirred at 0° C. for 1.5 hours, then warmed to room temperature (gas evolution observed) and stirred for 18 hours. The crude solution was transferred to a separating funnel and extracted with Et₂O (4×). The combined organics were extracted with aqueous NaHCO₃ (3×). The basic aqueous layer was cautiously acidified with 6N HCl and extracted with CH₂Cl₂ (3×). The CH₂Cl₂ extracts were dried (Na₂SO₄), filtered and concentrated in vacuo to give the crude sub-title compound (26.5 g, 46% from 3-trifluoromethoxybenzoic acid) as a solid that was used in the next step without further purification.

¹H NMR (300 MHz, CD₃OD): Λ 7.98 (s, 1H), 7.83 (s, 1H), 7.58 (s, 1H)

### (v) 3-Chloro-5-trifluoromethoxybenzyl alcohol

To a solution of 3-chloro-5-trifluoromethoxybenzoic acid (22.5 g, 93.5 mmol; see step (iv) above) in anhydrous THF (1200 mL) under a N₂ atmosphere at room temperature was added a solution of BH₃-THF complex (140 mL of 1M in THF; 140.3 mmol). The solution was refluxed for 2 h, cooled to room temperature and stirred for 18 hours, quenched cautiously with H₂O and concentrated in vacuo to remove most of the THF. The residue was diluted with EtOAc and the organics were washed with brine (3×), dried (Na₂SO₄), filtered and concentrated in vacuo to give the crude sub-title compound (21.2 g, 100%) as an oil that was used without further purification.

¹H NMR (300 MHz, CDCl₃): Λ 7.33 (s, 1H), 7.17 (s, 1H), 7.14 (s, 1H), 4.72 (s, 2H), 2.05 (brs, 1H)

### (vi) 3-Chloro-5-trifluoromethoxybenzaldehyde

A solution of DMSO (16.1 g, 205.9 mmol) in anhydrous CH₂Cl₂ (300 mL) was cooled to −78° C. Oxalyl chloride (13.1 g, 103.0 mmol) was added slowly via a syringe (gas evolution was observed). The resulting solution was stirred at −78° C. for 15 minutes. A solution of 3-chloro-5-trifluoromethoxybenzyl alcohol (21.2 g, 93.6 mmol; see step (v) above) in CH₂Cl₂ (200 mL) was added via an addition funnel over a period of 15 minutes. The cloudy solution was stirred at −78° C. for 40 minutes and DIPEA (60.5 g, 468.0 mmol) was added via an addition funnel over 10 minutes. The resulting homogeneous solution was stirred at −78° C. for 1.5 hours, then warmed to room temperature and stirred 18 hours. The crude solution was concentrated in vacuo, the residue diluted with EtOAc and washed with H₂O (1×), 2N HCl (1×), brine (1×), aqueous NaHCO₃ (1×) and brine (1×). The organics were dried (Na₂SO₄), filtered and concentrated in vacuo to give the crude sub-title compound (19.9 g, 95%) which was used in the next step without further purification.

¹H NMR (300 MHz, CDCl₃): Λ 10.00 (s, 1H), 7.83 (s, 1H), 7.66 (s, 1H), 7.51 (s, 1H)

### (vii) Ph(3-Cl)(5-OCF₃)—(R,S)CH(OTMS)CN

To a solution of 3-chloro-5-trifluoromethoxybenzaldehyde (19.9 g, 88.6 mmol; see step (vi) above) in CH₂Cl₂ (600 mL) at 0° C. was added ZnI₂ (1.4 g, 4.4 mmol) and trimethylsilyl cyanide (9.7 g, 97.5 mmol). After stirring at 0° C. for 1.5 hours, and at room temperature for 2 hours, TLC analysis showed only the starting material. ZnI₂ was added portion-wise until the reaction proceeded (over 30.0 g of ZnI₂ was added in total). After stirring at room temperature for 18 h, the reaction was quenched with water and the organics were separated. The organics were dried (Na₂SO₄), filtered and concentrated in vacuo to give the crude sub-title compound (27.7 g, 96%) as a liquid that was used without further purification.

¹H NMR (300 MHz, CDCl₃): Λ 7.43 (s, 1H), 7.28 (s, 1H), 7.25 (s, 1H), 5.49 (s, 1H), 0.38 (s, 9H)

### (viii) Ph(3-Cl)(5-OCF₃)—(R,S)CH(OH)C(O)OH

A suspension of Ph(3-Cl)(5-OCF₃)—(R,S)CH(OTMS)CN (27.7 g, 85.6 mmol; see step (vii) above) in concentrated HCl (300 mL) was refluxed for 3 hours. The resulting brown heterogeneous mixture was cooled to room temperature and extracted with Et₂O (2×). The initial organics were extracted with 2N NaOH (2×), then the basic layer was acidified with 2N HCl and extracted with Et₂O. The Et₂O was dried (Na₂SO₄), filtered and concentrated in vacuo to give the crude sub-title compound (4.9 g, 21%). TLC analysis of the initial organics showed the sub-title compound was still present so the basic extraction/acidification was repeated using 6N NaOH to afford additional crude sub-title compound (2.8 g, 12%). TLC analysis of the initial organics showed the sub-title compound was still present so the organics were dried (Na₂SO₄) and concentrated in vacuo to give the sodium salt of the sub-title compound (18.3 g) as an oil. The salt was then re-dissolved in Et₂O and the organics acidified with 2N HCl and washed with brine. The resulting organics were dried (Na₂SO₄), treated with activated charcoal, filtered through Celite® and concentrated in vacuo to give the crude sub-title compound (14.3 g, 62%) as a solid that was used in the next step without further purification.

¹H NMR (300 MHz, CD₃OD): Λ 7.53 (s, 1H), 7.38 (s, 1H), 7.29 (s, 1H), 5.23 (s, 1H)

### (ix) Ph(3-Cl)(5-OCF₃)—(R)CH(OH)C(O)OH (a) and Ph(3-Cl)(5-OCF₃—(S)CH(OAc)C(O)OH (b)

A mixture of Ph(3-Cl)(5-OCF₃)—(R,S)CH(OH)C(O)OH (7.7 g, 28.5 mmol; see step (viii) above) and Lipase PS “Amano” (3.8 g) in MTBE (100 mL) and vinyl acetate (50 mL) was stirred at 60° C. for 26 hours. The reaction was cooled and filtered through Celite® and the filter cake washed with EtOAc. The combined organics were concentrated in vacuo. Flash chromatography on silica gel eluting with CHCl₃:MeOH:concentrated NH₄OH (6:3:1) afforded a mixture of the ammonium salts of sub-title compound (a) and sub-title compound (b) (6.7 g) and a pure sample of the ammonium salt of sub-title compound (a) (1.2.g) with less than 95% e.e. The respective fractions were dissolved in Et₂O and washed with 2N HCl (1×) and brine (1×), dried (Na₂SO₄), filtered and concentrated to give the corresponding carboxylic acids (6.7 g and 1.1 g respectively). These fractions were then separately re-submitted to the resolution conditions and re-purified as necessary via chromatography on silica gel eluting with CHCl₃:MeOH:concentrated NH₄OH (6:3:1 or 75:20:5 or 145:45:10) as needed. The purified sub-title compound (a) was acidified with aqueous HCl or aqueous citric acid prior to further use. The ammonium salt of sub-title compound (b) was used without characterization.

For sub-title compound (a)

¹H NMR (300 MHz, CD₃OD): Λ 7.53 (s, 1H), 7.38 (s, 1H), 7.29 (s, 1H), 5.23 (s, 1H)

¹³C NMR (75 MHz, CD₃OD): Λ 174.9, 150.9, 145.4, 136.3, 126.8, 122.0, 120.6, 118.9, 72.9

MS (m/z) 269 (M−1)⁻

### (x) Ph(3-Cl)(5-OCF₃)—(R)CH(OH)C(O)-Aze-Pab(Teoc)

A solution of Ph(3-Cl)(5-OCF₃)—(R)CH(OH)C(O)OH (0.73 g, 2.70 mmol; see step (ix) above) in DMF (40 mL) under a nitrogen atmosphere was cooled to 0° C. To the solution was added H-Aze-Pab(Teoc) (1.46 g, 3.24 mmol), collidine (0.82 g, 6.75 mmol) and PyBOP (1.83 g, 3.51 mmol). The solution was stirred at 0° C. for 2 h, warmed to room temperature and stirred 18 hours, quenched with water and concentrated in vacuo. The residue was diluted with EtOAc and washed with H₂O (1×), aqueous NaHCO₃ (1×), aqueous citric acid (1×) and brine (1×), dried (Na₂SO₄), filtered and concentrated in vacuo to give the crude sub-title compound. Flash chromatography on silica gel (2×) eluting with EtOAc:MeOH (30:1) then CH₂Cl₂:MeOH (93:7) afforded the sub-title compound (0.73 g, 43%) as a crushable foam.

¹H NMR (300 MHz, CD₃OD, complex mixture of rotamers): Λ 7.78-7.82 (d, 2H, J=8 Hz), 7.25-7.54 (m, 5H), 5.25 and 5.16 (s, 1H), 5.22 and 4.79 (m, 1H), 3.92-4.58 (m, 6H), 2.20-2.76 (m, 2H), 1.04-1.13 (m, 2H), 0.08 (s, 9H)

MS (m/z) 629 (M+1)⁺

### (xi) Ph(3-Cl)(5-OCF₃)—(R)CH(OH)C(O)-Aze-Pab

Trifluoroacetic acid (1.0 mL) was added to a stirred ice/water-cooled solution of Ph(3-Cl)(5-OCF₃)—(R)CH(OH)C(O)-Aze-Pab(Teoc) (101 mg; 160 μmol; see step (x) above), in methylene chloride (10 mL). The cooling bath was removed after 1 hour. After 1.5 hours at room temperature, acetonitrile (30 mL) was added and the solvents were carefully removed under reduced pressure. The residue was dissolved in water and freeze dried to afford 90 mg (92%) of the title compound as its TFA salt.

MS (m/z) 483 (M−1)⁻; 485 (M+1)⁺

¹H NMR (300 MHz; CD₃OD): (complex due to diastereomers/rotamers): Λ 7.70-7.80 (m, 2H), 7.45-7.58 (m, 3H), 7.24-7.38 (m, 2H), 5.26 (s, 1H), 5.17 (m, 1H, minor rotamer), 4.82 (m, 1H, major rotamer), 4.35-4.6 (m, 3H), 4.22 (m, 1H, major rotamer), 3.92-4.12 (m, 2H, minor rotamer), 2.70 (m, 1H, minor rotamer), 2.55 (m, 1H, major rotamer), 2.30 (m, 1H, major rotamer), 2.16 (m, 1H, minor rotamer)

¹³C NMR (100 MHz; CD₃OD): (carbonyl and/or amidine carbons, rotamers): Λ 173.7, 173.4, 173.0, 172.8, 168.1

### EXAMPLE 7

### Ph(3-Cl)(5-OCF₃)—(R)CH(OH)C(O)-Aze-Pab(OMe)

HATU (71 mg; 0.19 mmol) was added to a stirred ice/water-cooled solution of Ph(3-Cl)(5-OCF₃)—(R)CH(OH)C(O)OH (39 mg; 0.14 mmol; see Example 6(ix) above) in DMF (3mL). After 30 minutes, a solution of H-Aze-Pab(OMe)×2HCl (69 mg; 0.21 mmol; see international patent application WO 00/42059) and 2,4,6-collidine (0.080 mL; 0.58 mmol) in DMF (1.5 mL) was added. The reaction mixture was left overnight and the temperature was allowed to rise slowly to ambient. The solvents were removed in vacuo and the crude product was purified using reverse-phase HPLC (acetonitrile: 0.1M aq. ammonium acetate) to afford, after freeze drying the appropriate fractions, the title compound (61 mg, 97%) as a colourless solid.

MS (m/z) 513 (M−1)⁻, 515 (M+1)⁺

¹H NMR (500 MHz; CD₃OD): Λ 7.97 (bt, 1H), 7.53 (d, 2H), 7.27 (t, 1H), 7.22 (d, 2H), 7.19 (t, 1H), 7.11 (t, 2H), 6.77 (s, 1H), 4.92 (s, 1H), 4.9 (bs, 3H), 4.81 (m, 2H), 4.40 (m, 2H), 4.09 (m, 1H) 3.87 (s, 3H), 2.58 (m, 1H), 2.37(m, 1H)

¹³C NMR (125 MHz; CD₃OD): (carbonyl and/or amidine carbons): Λ 171.8, 169.9, 156.8

### EXAMPLE 8

### Parallel Synthesis of Alkoxyamidines

This synthesis was performed in a 96-well Robbins block. To wells containing an appropriate amount of O-substituted hydroxylamine (specified below; all of which are commercially available or were prepared using well known literature procedures) was added a solution of Ph(3-Cl)(5-OCF₃)—(R)CH(OH)C(O)-Aze-Pab(Teoc) (10 mg; 17 μmol; see Example 6(x) above) in acetonitrile (1.0 mL). The block was sealed and the reaction mixture was rotated overnight in an oven at 60° C. After cooling and filtration, the solids were washed with acetonitrile (3×0.3 mL). The combined liquid fractions were concentrated in a vacuum centrifuge. The residue was partitioned between water (0.4 mL) and ethyl acetate (0.4 mL). After liquid-liquid extraction was finished, everything was filtered through a column of Hydromatrix™. After washing three times with ethyl acetate, the combined filtrates were concentrated in a vacuum centrifuge. Deprotection was performed by addition of methylene chloride (0.1 mL) and trifluroacetic acid (0.3 mL). After stirring at room temperature for 3 hours, the solvents were removed in vacuo. The residue was partitioned between aqueous saturated sodium hydrogen carbonate (0.5 mL) and ethyl acetate (0.5 mL). After extraction, filtration through Hydromatrix™ and concentration (vide infra) the residue was dissolved in isopropanol/water (7/3) (1 mL). About 2% of this solution was removed and diluted with isopropanol/water (7/3) (1 mL) for LC-MS analysis. After removal of the solvents in vacuo the solid residue was transferred to a 96-well plate using acetonitrile and ethyl acetate to dissolve the compound. The solvents were evaporated in a vacuum centrifuge to afford the following title compounds:
Ph(3-Cl)(5-OCF₃)—(R)CH(OH)C(O)-Aze-Pab(OCH₂-3-(5-Me-isoxazole)) (from 3-[(aminooxy)methyl]-5-methylisoxazole×HCl (18 mg; 0.11 mmol)). Yield: 3.64 mg (35%) (MS (m/z) 596 (M+1)⁺);
Ph(3-Cl)(5-OCF₃)—(R)CH(OH)C(O)-Aze-Pab(OCH₂-3-pyridine) (from 3-[(aminooxy)methyl]pyridine×2 HCl (19 mg; 96 μmol). Yield: 5.14 mg (50%) (MS (m/z) 592 (M+1)⁺);
Ph(3-Cl)(5-OCF₃)—(R)CH(OH)C(O)-Aze-Pab(OzBu) (from O-isobutyl hydroxylamine×HCl (17 mg; 140 μmol). Yield: 4.4 mg (45%). MS (m/z) 557 (M+1)⁺);
Ph(3-Cl)(5-OCF₃)—(R)CH(OH)C(O)-Aze-Pab(OEt) (from O-ethyl hydroxylamine×HCl (14 mg; 140 μmol). Yield: 4.04 mg (42%). MS (m/z) 529 (M+1)⁺);
Ph(3-Cl)(5-OCF₃)—(R)CH(OH)C(O)-Aze-Pab(OBn) (from O-benzylhydroxylamine×HCl (17 mg; 110 μmol). Yield: 3.22 mg (29%). MS (m/z) 591 (M+1)⁺);
Ph(3-Cl)(5-OCF₃)—(R)CH(OH)C(O)-Aze-Pab(OcHexyl) (from O-cyclohexyl hydroxylamine×HCl (15 mg; 99 μmol). Yield: 2.9 mg (26%). MS (m/z) 583 (M+1)⁺);
Ph(3-Cl)(5-OCF₃)—(R)CH(OH)C(O)-Aze-Pab(OcBu) (from O-cyclobutyl hydroxylamine×HCl (17 mg; 140 μmol). Yield: 3.3 mg (30%). MS (m/z) 555 (M+1)⁺);
Ph(3-Cl)(5-OCF₃)—(R)CH(OH)C(O)-Aze-Pab(OCH₂CH₂OPh(3-CF₃)) (from O-[2-[3-(trifluoromethyl)phenoxy]ethyl]hydroxylamine×HCl (24 mg; 93 μmol). Yield: 6.52 mg (46%). MS (m/z) 689 (M+1)⁺);
Ph(3-Cl)(5-OCF₃)—(R)CH(OH)C(O)-Aze-Pab(OBn(4-Cl)) (from O-(4-chlorobenzyl)hydroxylamine×HCl (16 mg; 82 μmol). Yield: 3.47 mg (29%). MS (m/z) 625 (M+1)⁺);
Ph(3-Cl)(5-OCF₃)—(R)CH(OH)C(O)-Aze-Pab(OBn(3-MeO)) (from O-(3-methoxybenzyl)hydroxylamine×HCl (18 mg; 94 μmol). Yield: 4.33 mg (36%). MS (m/z) 621 (M+1)⁺);
Ph(3-Cl)(5-OCF₃)—(R)CH(OH)C(O)-Aze-Pab(OBn(2-Br)) (from O-(2-bromobenzyl)hydroxylamine×HCl (23 mg; 96 μmol). Yield: 3.87 mg (30%). MS (m/z) 671 (M+1)⁺);
Ph(3-Cl)(5-OCF₃)—(R)CH(OH)C(O)-Aze-Pab(OBn(4-Me)) (from O-(4-methylbenzyl)hydroxylamine×HCl (14 mg; 81 μmol). Yield: 2.91 mg (25%). MS (m/z) 605 (M+1)⁺); and
Ph(3-Cl)(5-OCF₃)—(R)CH(OH)C(O)-Aze-Pab(O-4-heptyl) (from O-(4-heptyl)hydroxylamine×HCl (15 mg; 89 μmol). Yield: 17 mg (100%). MS (m/z) 599 (M+1)⁺).

### EXAMPLE 9

### Ph(3-Cl)(5-OCHF₂)—(S)CH(CH₂OH)C(O)-Aze-Pab×HOAc

### (i) 3-Chloro-5-methoxybenzoic acid

Magnesium turnings (Fluka purum for Grignard reactions) were pre-treated in the following way: The turnings were placed in a glass sintered funnel and 0.1 M of hydrochloric acid was poured onto them. The turnings were stirred with a glass rod for a few seconds and then the acid was washed away with 3 portions of water. Finally, the turnings were washed with 2 portions of acetone and bottled. Tetrahydrofuran (100 mL, 99.95%) was dried by adding RedAl (1 g, 70% wt. in toluene). Pre-treated magnesium turnings (5 g, 200 mmol) were placed in a round bottomed flask, and were flushed with nitrogen 3 times. Dichloroanisole (26 g, 146 mmol) was dissolved in THF (100 mL, RedAl-dried) and dibromoethane (1.8 g, 10 mmol) was added. The reaction mixture was flushed with nitrogen and then refluxed for 2 hours. Heating was interrupted and dry ice (10 g) was added portionwise over 2 minutes. When all of the dry ice was dissolved, the reaction mixture was poured into ice containing hydrochloric acid (400 mL, 2 M). Extractive work up (ether, 300 mL) gave 11.2 g, 60.2 mmol (yield: 41%) of the sub-title compound.

¹H-NMR (500 MHz; acetone-d₆): Λ 7.57 (m, 1H), 7.49 (m, 1H), 7.23 (m, 1H), 3.91 (s, 3H)

### (ii) 3-Chloro-5-hydroxybenzoic acid

Alumina (1.65 g, 60 mmol) and iodine (21 g, 82 mmol) were refluxed in toluene (200 mL) for 2 hours. Then, 3-chloro-5-methoxybenzoic acid (11.2 g, 60.2 mmol; see step (i) above) dissolved in toluene (50 mL) was added, together with tetrabutylammonium iodide (1.5 g, 4 mmol), and the mixture was refluxed for another 2 hours. After cooling to ambient temperature, extractive work up gave 8.7 g, 50 mmol (yield: 83%) of the sub-title compound.

¹H-NMR (300 MHz; acetone-d₆): Λ 9.27 (s, 1H), 7.48 (m, 1H), 7.44 (m, 1H), 7.11 (m, 1H)

### (iii) 3-Chloro-5-difluoromethoxybenzoic acid

3-Chloro-5-hydroxybenzoic acid (6.4 g, 37.2 mmol; see step (ii) above) dissolved in chloroform (200 mL) was transferred to a 500 mL three-necked round-bottomed flask fitted with a dry ice condenser and a gas inlet tube. Sodium hydroxide (100 mL, 5 M) was added and with vigorous stirring. Chlorodifluoromethane (Freon 22; 25 g, 290 mmol) was is added portionwise through the gas inlet tube at ambient temperature. After 2 hours, the reaction was complete. Extractive work up gave 6.2 g, 28 mmol (yield: 75%) of the sub-title compound.

¹H-NMR (500 MHz; acetone-d₆): Λ 7.87 (m, 1H), 7.74 (m, 1H), 7.54 (m, 1H), 7.19 (t, 1H, J_{H-F} 73 Hz)

### (iv) 3-Chloro-5-difluoromethoxy-N-methoxy-N-methylbenzamide

3-Chloro-5-difluoromethoxybenzoic acid (1.8 g, 8 mmol; see step (iii) above) and oxalyl chloride (1.5 g, 11.8 mmol) were dissolved in methylene chloride (50 mL). DMF (2 drops) was added and the reaction mixture was stirred at ambient temperature for 30 minutes. Then, N,O-dimethylhydroxylamine (1 g, 10.2 mmol) and triethylamine (3 g, 30 mmol) were added and after another 10 minutes stirring at ambient temperature, the reaction mixture was concentrated at reduced pressure. The residue was taken up in ether (100 mL) and water (50 mL). After separation, the organic phase was washed with brine, dried over sodium sulphate, filtered and concentrated. This residue was chromatographed on silica (hexane/ethyl acetate 2:1) which gave 2 g, 7.5 mmol (93%) of the sub-title compound.

¹H-NMR (400 MHz; CDCl₃): Λ 7.54 (m, 1H), 7.37 (m, 1H), 7.27 (m, 1H), 6.53 (t, 1H, J_{H-F} 73 Hz)

### (v) 3-Chloro-5-difluoromethoxyacetophenone

3-Chloro-5-difluoromethoxy-N-methoxy-N-methylbenzamide (2 g, 7.5 mmol; see step (iv) above) was dissolved in ether (100 mL) and cooled under nitrogen to −70° C. Methyllithium (7 mL, 11 mmol, 1.6 M in ether) was added dropwise with a syringe to the stirred reaction mixture over 1 minute. The dry ice bath was removed and the mixture was allowed to reach ambient temperature before the reaction was quenched with ammonium chloride solution (50 mL, 5% NH₄Cl in water). The organic phase was washed with brine, dried over sodium sulphate, filtered and concentrated at reduced pressure. The residue was chromatographed on silica (hexane:ethyl acetate 2:1) which gave 1.5 g, 6.8 mmol (yield: 90%) of the sub-title compound.

¹H-NMR (600 MHz; CDCl₃): Λ 7.77 (m, 1H), 7.59 (m, 1H), 7.35 (m, 1H), 6.56 (t, 1H, J_{H-F} 73 Hz), 2.60 (s, 3H)

### (vi) 3-Chloro-5-difluoromethoxyphenylacetic acid methyl ester

3-Chloro-5-difluoromethoxyacetophenone (1.5 g, 6.8 mmol; see step (v) above) was dissolved in methylene chloride (200 mL). Thallium(III) nitrate×3MeOH on K-10 montmorillonite (6 g, 10 mmol (ca 0.6 mmol/g); see J. Am. Chem. Soc., 98, 6750 (1976)) was added and the mixture was stirred at ambient temperature for 20 hours. The mixture was filtered and the filtrate was washed with sodium bicarbonate (100 mL, 0.5 M), dried over sodium sulphate, filtered and concentrated at reduced pressure. The residue was chromatographed on silica (hexane/ethyl acetate 2:1) which gave 1 g, 4 mmol (yield: 56%) of the sub-title compound.

¹H-NMR (500 MHz; CDCl₃): Λ 7.14 (m, 1H), 7.06 (m, 1H), 6.96 (m, 1H), 6.50 (t, 1H, J_{H-F} 73 Hz), 3.72 (s, 3H), 3.60 (s, 1H)

### (vii) 1-Formyl(3-chloro-5-difluoromethoxyphenyl)acetic acid methyl ester

3-Chloro-5-difluoromethoxyphenylacetic acid methyl ester (1 g, 4 mmol; see step (vi) above) and methyl formate (1 g, 16 mmol) were dissolved in ether (100 mL) and cooled in an ice-bath (ca. 2° C.). Then, finely cut sodium (180 mg, 7.8 mmol) and methanol (1 mL) were added and the mixture was left in the ice-bath with stirring overnight. Water (100 mL) was added carefully and the phases were separated. The water containing phase was acidified with hydrochloric acid (2 M) to pH 1 and extracted with ether (2×100 mL). The extract was dried over sodium sulphate, filtered and concentrated at reduced pressure. The residue was chromatographed on silica (hexane:ethyl acetate (1:1)) which gave 400 mg, 1.4 mmol (yield: 36%) of the sub-title compound.

¹H-NMR (400 MHz): Λ 12.10 (d, 1H), 7.32 (d, 1H), 7.11 (m, 1H), 7.07 (m, 1H), 6.94 (m, 1H), 6.51 (t, 1H, J_{F-H} 73), 3.83 (s, 3H)

### (viii) 3-Chloro-5-difluoromethoxytropic acid

I-Formyl(3-chloro-5-difluoromethoxyphenyl)acetic acid methyl ester (400 mg, 1.4 mmol; see step (vii) above) was dissolved in THF:methanol (50 mL, 9:1). Sodium borohydride was added and the mixture was stirred at ambient temperature for 30 minutes. Water was added and the mixture was concentrated to produce an aqueous suspension, which was taken up in ethyl acetate and water. The phases were separated and the organic phase was washed with sodium chloride (15% in water), dried over sodium sulphate, filtered and concentrated at reduced pressure. The residue was dissolved in methanol (30 mL) and hydrolyzed with sodium hydroxide (1 mL, 10 M) at ambient temperature for 10 minutes. Extractive work up gave 180 mg, 0.68 mmol (yield: 48%) of the sub-title compound.

¹H-NMR (500 MHz; CDCl₃): Λ 7.18 (m, 1H), 7.10 (m, 1H), 7.00 (m, 1H), 6.50 (t, 1H, J_{F-H} 73), 4.11 (m, 1H), 3.90 (m, 1H), 3.84 (m, 1H)

### (ix) Ph(3-Cl)(5-OCHF₃)—(S)CH(CH₂OH)C(O)-Aze-Pab×HOAc

3-Chloro-5-difluoromethoxytropic acid (180 mg, 0.7 mmol; see step (viii) above), H-Aze-Pab(Teoc)×HCl (450 mg, 1 mmol) and PyBOP (530 mg, 1 mmol) were dissolved in DMF (10 mL), whereafter DIPEA (550 mg, 3.9 mmol) was added. The mixture was stirred at ambient temperature for 1 h before it was diluted with brine (20 mL, 15% NaCl) and extracted with ethyl acetate (40 mL). The extract was dried over sodium sulphate, filtered and evaporated to dryness. The residue was dissolved in methylene chloride (5 mL) and trifluroacetic acid (5 mL) was added. After 1 h at ambient temperature, the mixture of diastereomers was evaporated to dryness and the residue was chromatographed on a reverse phase column (acetonitrile:water (30:70), buffer: ammonium acetate 0.1 M). Freeze drying gave 36 mg, 0.067 mmol (yield: 10.4%) of the title compound.

MS (ES) 481 (M+1)⁺

¹H-NMR (400 MHz; CDCl₃): Λ 7.77 (d, 2H), 7.57 (d, 2H), 7.30 (m, 1H), 7.13 (m, 2H), 6.87 (t, 1H, J_{F-H} 73 Hz), 4.76 (m, 1H), 4.55 (s, 2H), 4.37 (m, 1H), 4.03 (m, 2H), 3.82 (m, 1H), 3.72 (m, 1H), 2.53 (m, 1H), 2.28 (m, 1H), 1.92 (s, 1,5H)

¹³C-NMR (100 MHz; CD₃OD): (carbonyl and/or amidine carbons) Λ 172.3, 171.9, 167.2

### EXAMPLE 10

### Ph(3-Cl)(5-OCF₃)—(S)CH(CH,OH)C(O)-Aze-Pab×TFA

### (i) 3-Chloro-5-trifluoromethoxybenzyl mesylate

To a solution of 3-chloro-5-trifluoromethoxybenzyl alcohol (6.1 g, 26.9 mmol; see Example 6(v) above) in CH₂Cl₂ (250 mL) at 0° C. under a nitrogen atmosphere was added DIPEA (4.2 g, 32.3 mmol) and methanesulfonyl chloride (3.4 g, 29.6 mmol). The solution was stirred at 0° C. for 1.5 hours and quenched with H₂O. The organics were separated and then washed with H₂O (1×), 1N HCl (1×), H₂O (1×) and aqueous NaHCO₃ (1×) and then dried (Na₂SO₄), filtered and concentrated to afford the sub-title compound (8.2 g, 99%) as an oil.

¹H NMR (300 MHz, CDCl₃): Λ 7.37 (s, 1H), 7.28 (s, 1H), 7.18 (s, 1H) 5.23 (s, 2H), 3.07 (s, 3H)

### (ii) 3-Chloro-5-trifluoromethoxybenzyl cyanide

To a solution of 3-chloro-5-trifluoromethoxybenzyl mesylate (8.2 g, 26.8 mmol; see step (i) above) in DMSO (50 mL) was added sodium cyanide (2.6 g, 53.6 mmol). The resulting heterogeneous solution was warmed to 50° C. and sonicated for 1 hour. The reaction was cooled and partitioned between Et₂O and H₂O. The organics were washed with H₂O (2×) and brine (2×). The combined aqueous phases were extracted with Et₂O (1×). The combined organics were dried (Na₂SO₄), filtered and concentrated under a low heat and partial vacuum to afford the sub-title compound (6.3 g, 100%) as a reddish volatile oil which was used in the next step without further purification.

¹H NMR (300 MHz, CDCl₃) Λ 7.32 (s, 1H), 7.24 (s, 1H), 7.12 (s, 1H), 3.78 (s, 2H)

### (iii) 3-Chloro-5-trifluoromethoxyphenylacetic acid

To a solution of 3-chloro-5-trifluoromethoxybenzyl cyanide (6.3 g, 26.7 mmol; see step (ii) above) in 2-propanol (100 mL) was added water (200 mL) and potassium hydroxide (7.5 g, 133.5 mmol). The solution was refluxed for 18 h, cooled to room temperature, and the 2-propanol was removed in vacuo. The aqueous phase was washed with CH₂Cl₂ (2×) and the washings discarded. The basic aqueous phase was acidified with 2N HCl and extracted with CH₂Cl₂ (3×). The CH₂Cl₂ extracts were dried (Na₂SO₄), filtered and concentrated in vacuo to afford the sub-title compound (5.2 g, 76%) as an oil which was used in the next step without further purification.

¹H NMR (300 MHz, CDCl₃): Λ 7.25 (s, 1H), 7.19 (s, 1H), 7.08 (s, 1H), 3.68 (s, 2H)

### (iv) Ethyl 3-chloro-5-trifluoromethoxyphenylacetate

To a solution of 3-chloro-5-trifluoromethoxyphenylacetic acid (5.2 g, 20.4 mmol; see step (iii) above) in EtOH (600 mL) was added sulfuric acid (several drops). The solution was refluxed for 18 h, cooled to room temperature, neutralized with solid NaHCO₃ and the EtOH removed in vacuo. The residue was diluted with EtOAc then washed with H₂O (1×), aqueous NaHCO₃ (1×) and brine (1×). The organics were dried (Na₂SO₄), filtered and concentrated in vacuo to afford the sub-title compound (5.5 g, 96%) as an oil which was used in the next step without further purification.

¹H NMR (300 MHz, CDCl₃) Λ 7.24 (s, 1H), 7.16 (s, 1H), 7.07 (s, 1H), 4.13-4.22 (q, J=8 Hz, 2H), 3.63 (s, 2H), 1.24-1.32 (t, J=8 Hz, 3H)

### (v) Ph(3-Cl)(5-OCF₃)—(R,S)CH(CHO)C(O)OEt

To a solution of ethyl 3-chloro-5-trifluoromethoxyphenylacetate (4.5 g, 15.9 mmol; see step (iv) above) in anhydrous THF (400 mL) under a nitrogen atmosphere at less than 0° C. (ice-MeOH bath) was added sodium ethoxide (4.5 g, 63.6 mmol). The cold solution was stirred for 40 minutes and ethyl formate (8.1 g, 111.3 mmol) was added. The solution was stirred at 0° C. for 30 minutes, warmed to room temperature and stirred for 2 hours. Then, the THF was removed in vacuo. The residue was diluted with Et₂O and extracted with H₂O (1×) and 0.5M NaOH (3×). The aqueous extracts were acidified with 2N HCl and extracted with CH₂Cl₂ (3×). The combined organics were dried (Na₂SO₄), filtered and concentrated in vacuo to afford the crude sub-title compound (3.9 g). Flash chromatography on silica gel eluting with Hex:EtOAc (4:1) afforded the sub-title compound (3.0 g, 61%) as an oil.

¹H NMR (300 MHz, CDCl₃, mixture of isomers): A 12.30 and 12.25 (s, 1H), 7.39 and 7.34 (s, 1H), 7.21 (s, 1H), 7.17 (s, 1H), 7.08 (s, 1H), 4.27-4.37 (q, J=8 Hz, 2H), 1.28-1.38 (t, J=8 Hz, 3H)

### (vi) Ph(3-Cl)(5-OCF₃)—(R,S)CH(CH₂OH)C(O)OEt

To a solution of Ph(3-Cl)(5-OCF₃)—(R,S)CH(CHO)C(O)OEt (3.0 g, 9.66 mmol; see step (v) above) in MeOH (200 mL) at −10° C. (ice-MeOH bath) was added sodium borohydride (0.7 g, 19.32 mmol) portion-wise over 5 min. The solution was stirred at −10° C. for 45 minutes and additional sodium borohydride (0.4 g) was added. After another 15 minutes, the reaction was quenched with aqueous ammonium chloride, made weakly acidic with 2N HCl and the MeOH was removed in vacuo. The residue was diluted with EtOAc and washed with H₂O (1×), aqueous NaHCO₃ (1×) and brine (1×). The organics were dried (Na₂SO₄), filtered and concentrated in vacuo to afford the crude sub-title compound. Flash chromatography on silica gel eluting with Hex:EtOAc (5:1) afforded the sub-title compound (2.0 g, 66%) as an oil.

¹H NMR (300 MHz, CDCl₃): ζ 7.26 (s, 1H), 7.19 (s, 1H), 7.07 (s, 1H), 4.16-4.28 (m, 2H), 4.04-4.15 (m, 1H), 3.76-3.94 (m, 2H), 2.33 (t, J=6 Hz, 1H), 1.18-1.30 (t, J=8 Hz, 3H)

### (vii) Ph(3-Cl)(5-OCF₃)—(R,S)CH(CH₂OH)C(O)OH

To a solution of Ph(3-Cl)(5-OCF₃)—(R,S)CH(CH₂OH)C(O)OEt (2.0 g, 6.24 mmol; see step (vi) above) in THF (50 mL) and H₂O (25 mL) was added lithium hydroxide monohydrate (0.5 g, 12.48 mmol). The solution was stirred at room temperature for 1 hour and the THF was removed in vacuo. The residue was diluted with H₂O then washed with CHCl₃ (2×) and the washings discarded. The basic aqueous layer was acidified with 2N HCl and extracted with CHCl₃ (4×). The CHCl₃ extracts were dried (Na₂SO₄), filtered and concentrated in vacuo to afford the crude sub-title compound (1.5 g) as an oil. Flash chromatography on silica gel eluting with CHCl₃:MeOH:concentrated NH₄OH (gradient from 7.0:2.5:0.5 to 6:3:1) afforded the ammonium salt of the sub-title compound (1.1 g). The ammonium salt was partitioned between 1N HCl and CHCl₃. The organics were dried (Na₂SO₄), filtered and concentrated in vacuo to afford the sub-title compound (also known as 3-chloro-5-trifluoromethoxytropic acid) as an oil (1.1 g, 62%).

¹H NMR (300 MHz, CD₃OD): Λ 7.41 (s, 1H), 7.27 (s, 1H), 7.24 (s, 1H), 4.03 (m, 1H), 3.75-3.87 (m, 2H)

### (viii) Ph(3-Cl)(5-OCF₃)—(S)CH(CH₂OH)C(O)-Aze-Pab(Teoc) (a) and Ph(3-Cl)(5-OCF₃)—(R)CH(CH₂OH)C(O)-Aze-Pab(Teoc) (b)

To a solution of Ph(3-Cl)(5-OCF₃)—(R,S)CH(CH₂OH)C(O)OH (0.65 g, 2.28 mmol; see step (vii) above) in DMF at less than 0° C. (ice-MeOH bath) was added H-Aze-Pab(Teoc) (0.90 g, 2.39 mmol), collidine (0.71 g, 5.70 mmol) and PyBOP (1.31 g, 2.51 mmol). The resulting solution was stirred at less than 0° C. for 1 h, warmed to room temperature and stirred for 1 hour. The DMF was then removed in vacuo. The residue was diluted with EtOAc and washed with dilute aqueous HCl (1×), brine (1×), aqueous NaHCO₃ (1×) and brine (1×). The organics were dried (Na₂SO₄), filtered and concentrated in vacuo to afford the crude sub-title compound (2.1 g) as a mixture of diastereomers. Flash chromatography (3×) on silica gel eluting first with EtOAc:MeOH (95:5) then with CH₂Cl₂:MeOH (97:3) and last with CH₂Cl₂:MeOH (95:5) afforded the sub-title compounds diastereomer (a) (0.51 g, 35%) and diastereomer (b) (0.45 g, 31%) as crushable foams.

For sub-title compound diastereomer (a)

¹H NMR (300 MHz, CD₃OD, complex mixture of rotamers) Λ 7.79-7.85 (d, J=8 Hz, 2H), 7.22-7.49 (m, 5H), 5.17-4.77 (m, 1H), 4.53-4.18 (m, 4H), 3.58-4.11 (m, 5H), 2.47-2.73 (m, 1H), 2.11-2.34 (m, 1H), 1.08-1.12 (m, 2H), 0.07 (s, 9H) MS (m/z) 643 (M+1)⁺

### (ix) Ph(3-Cl)(5-OCF₃)—(S)CH(CH₂OH)C(O)-Aze-Pab×TFA

Ph(3-Cl)(5-OCF₃)—(S)CH(CH₂OH)C(O)-Aze-Pab(Teoc), (78 mg, 0.121 mmol; see step (viii) above—diastereomer (a)), was dissolved in 5 mL of trifluoroacetic acid. After 10 minutes, the reaction was over and the solvent was evaporated. The residue was freeze dried from water and acetonitrile to give the desired product. Yield: 70 mg (94%).

MS (m/z) 483 (M−1)⁻; 485 (M+1)⁺

¹H-NMR(400 MHz; D₂O) rotamers 1:1:δ8.83 (bt, 1H), 7.79 (d, 1H), 7.72 (d, 1H), 7.54 (d, 1H), 7.43 (d, 2H), 7.35 (m, 1H, rotamer), 7.28 (m, 1H, rotamer), 7.20 (m, 1H, rotamer), 7.05 (m, 1H, rotamer), 5.22 (m, 1H, rotamer), 4.83 (m, 1H, rotamer), 4.57 (m, 2H, rotamer), 4.38 (m, 2H, rotamer), 4.3-3.7 (m, 5H), 2.77 (m, 1H, rotamer), 2.55 (m, 1H, rotamer), 2.27 (m, 1H)

¹³C-NMR (100 MHz; D₂O): (carbonyl and/or amidine carbons, rotamers) δ 172.9, 172.2, 172.0, 171.8, 166.9

### EXAMPLE 11

### Ph(3-Cl)(5-OCF₃)—(S)CH(CH₂OH)C(O)-Aze-Pab(OMe)

### (i) Ph(3-Cl)(5-OCF₃)—(S)CH(CH₂OH)C(O)-Aze-Pab(OMe, Teoc)

Ph(3-Cl)(5-OCF₃)—(S)CH(CH₂OH)C(O)-Aze-Pab(Teoc) (100 mg, 0.155 mmol; see Example 10(viii) above), was dissolved in 12 mL of tetrahydrofuran. O-Methylhydroxylamine hydrochloride (44 mg, 0.53 mmol), was added and the reaction was heated at 50° C. overnight. The reaction mixture was evaporated and the residue purified by preparative HPLC (CH₃CN/0.1 M NH₄OAc (70/30)). The pertinent fractions were evaporated and the residue dissolved in a small amount of acetonitrile and water and freeze dried. The freeze drying was repeated once. Yield: 80 mg (76%) of pure material.

¹H-NMR(400 MHz; CD₃OD) rotamers: δ 7.5-7.4 (m, 3H), 7.35-7.2 (m, 4H), 5.15 (m, 1H, minor rotamer), 4.74 (m, 1H, major rotamer), 4.5-4.25 (m, 3H), 4.2-3.95 (m, 4H), 3.91 (b, 3H), 3.9-3.6 (m, 2H), 2.63 (m, 1H, minor rotamer), 2.50 (m, 1H, major rotamer), 2.3-2.1 (m, 1H), 0.95 (m, 2H), 0.02 (s, 9H, major rotamer), 0.01 (s, 9H, minor rotamer)

### (ii) Ph(3-Cl)(5-OCF₃)—(S)CH(CH₂OH)C(O)-Aze-Pab(OMe)

Ph(3-Cl)(5-OCF₃)—(S)CH(CH₂OH)C(O)-Aze-Pab(OMe, Teoc), (80 mg, 0.12 mmol; see step (i) above), was dissolved in 1 mL of methylene chloride and cooled in an ice bath. Trifluoroacetic acid, 3 mL, was added and the reaction flask was kept in the ice bath for two hours. The mixture was evaporated and dissolved in ethyl acetate and washed three times with NaHCO₃ (aq) then with water and brine. The organic phase was dried (Na₂SO₄), filtered and evaporated. The residue was freeze dried from a small amount of acetonitrile and water. Yield: 60 mg (95%) of pure title product.

MS (m/z) 528 (M−1)⁻; 531 (M+1)⁺

¹H-NMR(500 MHz; CD₃OD) rotamers: δ 7.65-7.55 (m, 3H, rotamers), 7.45 (m, 1H, major rotamer), 7.4-7.2 (m, 4H), 5.15 (m, 1H, minor rotamer), 4.74 (m, 1H, major rotamer), 4.5-4.3 (m, 3H), 4.05-3.95 (m, 2H), 3.85 (m, 1H, major rotamer), 3.82 (s, 3H, major rotamer), 3.81 (s, 3H, minor rotamer), 3.73 (m, 1H, major rotamer), 3.67 (m, 1H, minor rotamer), 3.62 (m, 1H, minor rotamer), 2.63 (m, 1H, minor rotamer), 2.50 (m, 1H, major rotamer), 2.24 (m, 1H, major rotamer), 2.16 (m, 1H, minor rotamer)

¹³C-NMR (125 MHz; CD₃OD): (carbonyl and/or amidine carbons, rotamers) δ 174.0, 173.2, 172.7, 172.6, 155.1

### EXAMPLE 12

### Ph(3-Cl)(5-OCHF₂)—(R)CH(OH)C(O)-Aze-Pab(OMe)

### (i) Ph(3-Cl)(5-OCHF₂)—(R)CH(OH)C(O)-Aze-Pab(OMe, Teoc)

Ph(3-Cl)(5-OCHF₂)—(R)CH(OH)C(O)-Aze-Pab(Teoc) (0.40 g, 0.65 mmol; see Example 1(ix) above), was dissolved in 20 mL of acetonitrile and 0.50 g (6.0 mmol) of O-methyl hydroxylamine hydrochloride was added. The mixture was heated at 70° C. for 2 h. The solvent was evaporated and the residue was partitioned between water and ethyl acetate. The aqueous phase was extracted twice more with ethyl acetate and the combined organic phase was washed with water, brine, dried (Na₂SO₄), filtered and evaporated. Yield: 0.41 g (91%).

¹H-NMR (400 MHz; CDCl₃): δ 7.83 (bt, 1H), 7.57 (bs, 1H), 7.47 (d, 2H), 7.30 (d, 2H), 7.20 (m, 1H), 7.14 (m, 1H), 7.01 (m, 1H), 6.53 (t, 1H), 4.89 (s, 1H), 4.87 (m, 1H), 4.47 (m, 2H), 4.4-4.2 (b, 1H), 4.17-4.1 (m, 3H), 3.95 (s, 3H), 3.67 (m, 1H), 2.68 (m, 1H), 2.42 (m, 1H) 0.97 (m, 2H), 0.01 (s, 9H).

### (ii) Ph(3-Cl)(5-OCHF₂)—(R)CH(OH)C(O)-Aze-Pab(OMe)

Ph(3-Cl)(5-OCHF₂)—(R)CH(OH)C(O)-Aze-Pab(OMe, Teoc) (0.40 g, 0.62 mmol; see step (i) above), was dissolved in 5 mL of TFA and allowed to react for 30 min. TFA was evaporated and the residue was partitioned between ethyl acetate and NaHCO₃ (aq.). The aqueous phase was extracted twice more with ethyl acetate and the combined organic phase was washed with water, brine, dried (Na₂SO₄), filtered and evaporated. The product was freeze dried from water/acetonitrile. No purification was necessary. Yield: 0.28 g (85%).

¹H-NMR (600 MHz; CDCl₃): δ 7.89 (bt, 1H), 7.57 (d, 2H), 7.28 (d, 2H), 7.18 (m, 1H), 7.13 (m, 1H), 6.99 (m, 1H), 6.51 (t, 1H), 4.88 (s, 1H), 4.87 (m, 1H), 4.80 (bs, 2H), 4.48 (dd, 1H), 4.43 (dd, 1H), 4.10 (m, 1H), 3.89 (s, 3H), 3.68 (m, 1H), 2.68 (m, 1H), 2.40 (m, 1H).

¹³C-NMR (125 MHz; CDCl₃): (carbonyl and/or amidine carbons, rotamers) δ 172.9, 170.8, 152.7, 152.6

MS (m/z) 495 (M−1)⁻, 497 (M+1)⁺

### EXAMPLE 13

### Ph(3-OCHF₂)—(R)CH(OH)C(O)-Aze-Pab×HOAc

Ph(3-Cl)(5-OCHF₂)—(R)CH(OH)C(O)-Aze-Pab(OMe) (13 mg, 0.026 mmol; see Example 12 above) was dissolved in abs. ethanol (5 mL) and 30 mg of 10% Pd/C was added. Finally acetic acid (5 μL) was added and the mixture was hydrogenated at atmospheric pressure for 20 h. The mixture was filtered through Celite®, evaporated, and purified by reversed phase HPLC (0.1 M aq. ammonium acetate/MeCN). The appropriate fractions were freeze-dried to afford the title compound as a white solid: 8.5 mg (66%).

¹H-NMR(400 MHz; CD₃OD) rotamers: δ 7.73-7.78 (m, 2H), 7.55 (d, 2H), 7.19-7.43 (m, 3H), 7.06-7.13 (m, 1H), 6.83 (t, 1H, J_{HF}=74 Hz, major rotamer), 6.81 (t, 1H, major rotamer), 5.20 (s, 1H, major rotamer), 5.19 (m, 1H, minor rotamer), 5.15 (s, 1H, minor rotamer), 4.78 (m, 1H, major rotamer), 4.4-4.6 (several peaks, 2H), 4.35 (m, 1H, major rotamer), 4.08 (m, 1H), 3.99 (m, 1H, minor rotamer), 2.70 (m, 1H, minor rotamer), 2.52 (m, 1H, major rotamer), 2.30 (m, 1H, major rotamer), 2.15 (m, 1H, minor rotamer), 1.89 (s, 3H).

¹³C-NMR (100 MHz; CD₃OD): (carbonyl and/or amidine carbons, rotamers) δ 173.7, 172.9, 168.3.

MS (m/z) 433 (M+1)⁺; 431 (M−1)⁻

### EXAMPLE 14

### Ph(3-OCF₃)—(R)CH(OH)C(O)-Aze-Pab×TFA

Ph(3-Cl)(5-OCF₃)—(R)CH(OH)C(O)-Aze-Pab×TFA (34 mg, 0.057 mmol, from Example 6) was dissolved in 5 mL of ethanol and 20 mg of 10% Pd/C was added. The mixture was hydrogenated at atmospheric pressure overnight. The mixture was filtered through Celite®, evaporated, and freeze dried from water/acetonitrile.

¹H-NMR(400 MHz; CD₃OD) rotamers: δ 7.8-7.7 (m, 2H), 7.55 (m, 2H), 7.5-7.2 (m, 4H), 5.24 (s, 1H, major rotamer), 5.23 (m, 1H, minor rotamer), 5.18 (s, 1H, minor rotamer), 4.77 (m, 1H, major rotamer), 4.6-4.45 (m, 2H), 4.36 (m, 1H, major rotamer), 4.08 (m, 1H), 3.99 (m, 1H, minor rotamer), 2.70 (m, 1H, minor rotamer), 2.52 (m, 1H, major rotamer), 2.30 (m, 1H, major rotamer), 2.15 (m, 1H, minor rotamer).

¹³C-NMR (100 MHz; CD₃OD): (carbonyl and/or amidine carbons, rotamers) δ 174.1, 173.9, 173.5, 172.9, 168.2.

¹⁹-F NMR (282 MHz; CD₃OD): −59.8 and −59.9 (3F, minor and major rotamer respectively), −77.4 (3F) indicates that the salt is TFA.

MS (m/z) 451.3 (M+1)⁺

### EXAMPLE 15

### Ph(3-Cl)(5-OCH₂CF₃)—(R)CH(OH)C(O)-Aze-Pab×TFA

### (i) 3-Chloro-5-trifluoroethoxybenzaldehyde

To a magnetically stirred solution of 3-chloro-5-hydroxybenzaldehyde (2.0 g, 12.8 mmol; see Example 1(ii) above) and potassium carbonate (2.3 g, 16.6 mmol) in DMF (35 mL) under nitrogen was added 2,2,2-trifluoroethyl p-toluenesulfonate (4.2 g, 16.6 mmol) at room temperature. The mixture was heated to 110° C. for 7 h and then stirred overnight at room temperature. The reaction was cooled to 0° C., poured into ice-cold 2 N HCl (100 mL) and extracted with EtOAc (2×75 mL). The combined organic extracts were washed with 0.5 N HCl (2×50 mL), dried (Na₂SO₄), filtered, and concentrated in vacuo. The brown oil was chromatographed on silica gel eluting with Hex:EtOAc (6:1) to afford the sub-title compound (1.9 g, 61%) as a yellow oil.

¹H NMR (300 MHz, CDCl₃) Λ 9.44 (s, 1H), 7.56 (s, 1H), 7.33 (s, 1H), 7.28 (s, 1H), 4.42 (q, J=8 Hz, 2H)

### (ii) Ph(3-Cl)(5-OCH₂CF₃)-(R,S)CH(OTMS)CN

To a solution of 3-chloro-5-trifluoroethoxybenzaldehyde (5.2 g, 21.7 mmol; see step (i) above) and zinc iodide (1.7 g, 5.4 mmol) in CH₂Cl₂ (200 mL) under nitrogen was added trimethylsilyl cyanide (4.3 g, 43.3 mmol) dropwise via syringe at 0° C. The mixture was stirred at 0° C. for 3 h then diluted with H₂O (150 mL). The organic layer was separated, dried (Na₂SO₄), filtered, and concentrated in vacuo to afford the sub-title compound (6.9 g, 95%) as a yellow oil which was used without further purification.

¹H NMR (300 MHz, CDCl₃) Λ 7.27 (s, 1H), 6.98 (s, 2H), 5.44 (s, 1H), 4.38 (q, J=8 Hz, 2H), 0.30 (s, 9 H)

### (iii) Ph(3-Cl)(5-OCH₂CF₃)—(R,SCH(OH)C(O)OH

Concentrated hydrochloric acid (170 mL) was added to Ph(3-Cl)(5-OCH₂CF₃)—(R,S)CH(OTMS)CN (6.9 g, 20.4 mmol; see step (ii) above) and stirred at 100° C. for 1 h. After cooling to room temperature, the reaction was further cooled to 0° C. and basified slowly with 3 N NaOH (300 mL). This mixture was washed with Et₂O (2×100 mL) and the aqueous layer was acidified with 2 N HCl (50 mL). The aqueous layer was then extracted with EtOAc (2×100 mL), dried (Na₂SO₄), filtered, and concentrated in vacuo to afford the sub-title compound (5.3 g, 92%) as a pale yellow oil which was used without further purification.

¹H NMR (300 MHz, CD₃OD) Λ 7.18 (s, 1H), 7.07 (s, 1H), 7.02 (s, 1H), 5.13 (s, 1H), 4.58 (q, J=8 Hz, 2H)

### (iv) Ph(3-Cl)(5-OCH₂CF₃)—(R)CH(OHC(O)OH (a) and Ph(3-Cl)(5-OCH₂CF₃)—(S)CH(OAc)C(O)OH (b)

A solution of Ph(3-Cl)(5-OCH₂CF₃)—(R,S)CH(OH)C(O)OH (7.06 g, 24.8 mmol; see step (iii) above) and Lipase PS “Amano” (4.30 g) in vinyl acetate (250 mL) and MTBE (250 mL) was heated at 70° C. under nitrogen for 40 h. The reaction was cooled to room temperature, the enzyme was removed by filtration washing with EtOAc, and the filtrate concentrated in vacuo. Chromatography on silica gel eluting with CHCl₃:MeOH:Et₃N (92:6:2) afforded the triethylamine salt of the sub-title compound (a) (3.02 g) as a yellow oil. The salt of sub-title compound (a) was dissolved in H₂O (150 mL), acidified with 2 N HCl and extracted with EtOAc (2×75 mL). The combined organic extracts were dried (Na₂SO₄), filtered, and concentrated in vacuo to yield the sub-title compound (a) (2.18 g) as an off-white solid. In addition, the triethylamine salt of sub-title compound (b) (4.73 g) was obtained from the column chromatography mentioned above.

Data for sub-title compound (a):

mp: 98-103° C.

¹H NMR (300 MHz, CD₃OD) Λ 7.18 (s, 1H), 7.07 (s, 1H), 7.02 (s, 1H), 5.13 (s, 1H), 4.58 (q, J=8 Hz, 2H).

¹³C NMR (75 MHz, CD₃OD) Λ 175.4, 159.6, 144.6, 136.2, 125.0 (q, J=277 Hz), 121.8, 115.9, 113.1, 73.3, 67.0 (q, J=35 Hz)

HPLC Analysis: 98.6%, >99% ee, Chiralcel OD Column (97:3:0.5 Hex:EtOH:TFA mobile phase)

[I]²⁵_{D}=−81.5° (c=1.0, MeOH)

APCI-MS: (M−1)=283 m/z

### (v) Ph(3-Cl)(5-OCH₂CF₃)—(R)CH(OH)C(O)-Aze-Pab(Teoc)

To a solution of Ph(3-Cl)(5-OCH₂CF₃)—(R)CH(OH)C(O)OH (0.50 g, 1.8 mmol; see step (iv) above (compound (a))) in DMF (20 mL) under nitrogen was added H-Aze-Pab(Teoc)×HCl (1.03 g, 2.3 mmol), PyBOP (1.01 g, 1.9 mmol), and DIPEA (0.57 g, 4.4 mmol) at 0° C. The reaction was stirred at 0° C. for 2 h and then at room temperature for 20 h. The mixture was concentrated in vacuo and the residue chromatographed twice on silica gel, eluting first with CHCl₃:EtOH (10:1) and then with EtOAc:EtOH (10:1) to afford the sub-title compound (0.55 g, 48%) as a crushable white foam.

mp: 90-95° C.

R_{f}=0.42 (10:1 CHCl₃:EtOH)

¹H NMR (300 MHz, CD₃OD, complex mixture of rotamers) Λ 7.78-7.81 (m, 2H), 7.38-7.41 (m, 2H), 7.12-7.16 (m, 1H), 7.00-7.06 (m, 2H), 5.09-5.22 and 4.75-4.79 (m, 2H), 3.94-4.61 (m, 8H), 2.09-2.75 (m, 2H), 1.04-1.11 (m, 2H), 0.70 (s, 9H)

APCI-MS: (M+1)=643 m/z

### (vi) Ph(3-Cl)(5-OCH₂CF₃)—(R)CH(OH)C(O)-Aze-Pab×TFA

Ph(3-Cl)(5-OCH₂CF₃)—(R)CH(OH)C(O)-Aze-Pab(Teoc) (0.066 g, 0.103 mmol; see step (v) above), was dissolved in 3 mL of TFA and allowed to react for 30 min. TFA was evaporated and the residue was freeze dried from water/acetonitrile to yield 0.060 g (94%) of the title compound as its TFA salt.

¹H-NMR (400 MHz; CD₃OD) rotamers: δ 7.8-7.7 (m, 2H), 7.6-7.5 (m, 2H), 7.2-7.0 (m, 3H), 5.21 (m, 1H, minor rotamer), 5.17 (s, 1H, major rotamer), 5.11 (s, 1H, minor rotamer), 4.81 (m, 1H, major rotamer), 4.6-4.4 (m, 4H), 4.37 (m, 1H, major rotamer), 4.16 (m, 1H, major rotamer), 4.06 (m, 1H, minor rotamer), 3.99 (m, 1H, minor rotamer), 2.70 (m, 1H, minor rotamer), 2.54 (m, 1H, major rotamer), 2.29 (m, 1H, major rotamer), 2.15 (m, 1H, minor rotamer)

¹³C-NMR (100 MHz; CD₃OD): (carbonyl and/or amidine carbons, rotamers) δ 172.2, 171.8, 171.7, 167.0.

MS (m/z) 499.3 (M+1)⁺

### EXAMPLE 16

### Ph(3-Cl)(5-OCH₂CF₃)—(R)CH(OH)C(O)-Aze-Pab(OMe)

To a solution of Ph(3-Cl)(5-OCH₂CF₃)—(R)CH(OH)C(O)OH (0.48 g, 1.7 mmol; see Example 15(iv) above (compound (a)) in DMF (20 mL) under nitrogen was added H-Aze-Pab(OMe)×2HCl (0.74 g, 2.2 mmol), PyBOP (0.97 g, 1.9 mmol), and DIPEA (0.55 g, 4.2 mmol) at 0° C. The reaction was stirred at 0° C. for 2 h and then at room temperature. for 20 h. The mixture was concentrated in vacuo and the residue chromatographed twice on silica gel, eluting first with CHCl₃:EtOH (10:1) and second with EtOAc:EtOH (10:1) to afford the title compound (0.62 g, 69%) as a crushable white foam.

mp: 75-80° C.

R_{f}=0.43 (10:1 CHCl₃:EtOH)

¹H NMR (300 MHz, CD₃OD, complex mixture of rotamers) Λ 7.57-7.60 (m, 2H), 7.32-7.36 (m, 2H), 7.13-7.17 (m, 1H), 7.00-7.06 (m, 2H), 5.09-5.19 and 4.74-4.80 (m, 2H), 3.93-4.62 (m, 6H), 3.81 (s, 3H), 2.10-2.73 (m, 2H)

APCI-MS: (M+1)=529 m/z

### EXAMPLE 17

### Ph(3-Cl)(5-OCH₂CHF₂)—(R)CH(OH)C(O)-Aze-Pab×TFA

### (i) 2,2-Difluoroethyl ester methanesulfonic acid

To a magnetically stirred solution of 2,2-difluoroethanol (1.52 g, 18.5 mmol) in CH₂Cl₂ (20 mL) under nitrogen was added triethylamine (5.61 g, 55.5 mmol) and methanesulfonyl chloride (2.54 g, 22.2 mmol) at 0° C. The mixture was stirred at 0° C. for 1.5 h, diluted with CH₂Cl₂ (50 mL), and washed with 2 N HCl (50 mL). The aqueous layer was extracted with CH₂Cl₂ (30 mL) and the combined organic extracts washed with brine (30 mL), dried (Na₂SO₄), filtered, and concentrated in vacuo to afford the sub-title compound (2.52 g, 85%) as a yellow oil which was used without further purification.

¹H NMR (300 MHz, CDCl₃) Λ 6.02 (tt, J=3 Hz, J=55 Hz, 1H), 4.39 (dt, J=3 Hz, J=13 Hz, 2H), 3.13 (s, 3H)

### (ii) 3-Chloro-5-difluoroethoxybenzaldehyde

To a solution of 3-chloro-5-hydroxybenzaldehyde (1.50 g, 9.6 mmol; see Example 1(ii) above) and potassium carbonate (1.72 g, 12.5 mmol) in DMF (10 mL) under nitrogen was added a solution of 2,2-difluoroethyl ester methanesulfonic acid (2.0 g, 12.5 mmol; see step (i) above) in DMF (10 mL) dropwise at room temperature. The mixture was heated to 100° C. for 6 h and then stirred overnight at room temperature. The reaction was cooled to 0° C., poured into ice-cold 2 N HCl (100 mL), and extracted with EtOAc (2×75 mL). The combined organic extracts were washed with 0.5 N HCl (2×50 mL), dried (Na₂SO₄), filtered, and concentrated in vacuo. The brown oil was chromatographed on silica gel eluting with Hex:EtOAc (5:1) to afford the sub-title compound (1.35 g, 64%) as a yellow oil.

¹H NMR (300 MHz, CDCl₃) Λ 9.92 (s, 1H), 7.52 (s, 1H), 7.31 (s, 1H), 7.22 (s, 1H), 6.12 (tt, J=3 Hz, J=55 Hz, 1H), 4.26 (dt, J=3 Hz, J=15 Hz, 2H)

### (iii) Ph(3-Cl)(5-OCH₂CHF₂)—(R,S)CH(OTMS)CN

To a solution of 3-chloro-5-difluoroethoxybenzaldehyde (1.35 g, 6.1 mmol; see step (ii) above) and zinc iodide (0.48 g, 1.5 mmol) in CH₂Cl₂ (50 mL) was added trimethylsilyl cyanide (1.21 g, 12.2 mmol) dropwise at 0° C. under nitrogen. The mixture was stirred at 0° C. for 3 h, then diluted with H₂O (50 mL). The organic layer was separated, dried (Na₂SO₄), filtered, and concentrated in vacuo to afford the sub-title compound (1.85 g, 95%) as a brown oil which was used without further purification.

¹H NMR (300 MHz, CDCl₃) Λ 7.13 (s, 1H), 6.94 (s, 2H), 6.10 (tt, J=3 Hz, J=55 Hz, 1H), 5.43 (s, 1H), 4.20 (dt, J=3 Hz, J=15 Hz, 2H), 0.28 (s, 9H)

### (iv) Ph(3-Cl)(5-OCH₂CHF₂)—(R,S)CH(OH)C(O)OH

Concentrated hydrochloric acid (60 mL) was added to Ph(3-Cl)(5-OCH₂CHF₂)—(R,S)CH(OTMS)CN (1.85 g, 5.8 mmol; see step (iii) above) and stirred at 100° C. for 1 h. After cooling to room temperature, the reaction was further cooled to 0° C., basified slowly with 3 N NaOH (˜180 mL) and washed with Et₂O (2×75 mL). The aqueous layer was acidified with 2 N HCl (20 mL) and extracted with EtOAc (2×75 mL). The combined organic extracts were dried (Na₂SO₄), filtered, and concentrated in vacuo to afford the sub-title compound (1.50 g, 97%) as a pale yellow solid which was used without further purification.

¹H NMR (300 MHz, CD₃OD) Λ 7.15 (s, 1H), 7.05 (s, 1H), 6.98 (s, 1H), 6.19 (tt, J=4 Hz, J=55 Hz, 1H), 5.12 (s, 1H), 4.25 (dt, J=4 Hz, J=17 Hz, 2H)

### (v) Ph(3-Cl)(5-OCH₂CHF₂)—(S)CH(OAc)C(O)OH (a) and Ph(3-Cl)(5-OCH₂CHF₂)—(R)CH(OH)C(O)OH (b)

A solution of Ph(3-Cl)(5-OCH₂CHF₂)—(R,S)CH(OH)C(O)OH (3.90 g, 14.6 mmol; see step (iv) above) and Lipase PS “Amano” (2.50 g) in vinyl acetate (140 mL) and MTBE (140 mL) was heated at 70° C. under nitrogen for 40 h. The reaction was cooled to room temperature, the enzyme removed by filtration washing with EtOAc, and the filtrate concentrated in vacuo. Chromatography on silica gel eluting with CHCl₃:MeOH:Et₃N (92:6:2) afforded the triethylamine salt of the subtitle compound (a) as a yellow oil. In addition, the triethylamine salt of the sub-title compound (b) (1.47 g) was obtained and the salt was dissolved in H₂O (100 mL), acidified with 2 N HCl and extracted with EtOAc (2×75 mL). The combined organic extracts were dried (Na₂SO₄), filtered, and concentrated in vacuo to yield the sub-title compound (b) (1.00 g) as an off-white solid.

Data for sub-title compound (b):

mp: 103-106° C.

R_{f}=0.39 (90:8:2 CHCl₃:MeOH:Et₃N)

¹H NMR (300 MHz, CD₃OD) Λ 7.13 (s, 1H), 7.04 (s, 1H), 6.97 (s, 1H), 6.17 (tt, J=4 Hz, J=55 Hz, 1H), 5.12 (s, 1H), 4.24 (dt, J=4 Hz, J=8 Hz, 2H).

¹³C NMR (75 MHz, CD₃OD) Λ 175.5, 160.3, 144.5, 136.1, 121.3, 115.7, 115.3, (t, J=240 Hz), 112.9, 73.4, 68.6 (t, J=29 Hz)

HPLC Analysis: 96.2%, >95.0% ee, ChiralPak AD Column (95:5:0.5 Hex:EtOH:TFA mobile phase)

[I]²⁵_{D}=−84.0° (c=0.85 MeOH)

APCI-MS: (M−1)=265 m/z

### (vi) Ph(3-Cl)(5-OCH₂CHF₂)—(R)CH(OH)C(O)-Aze-Pab(Teoc)

To a solution of Ph(3-Cl)(5-OCH₂CHF₂)—(R)CH(OH)C(O)OH (0.35 g, 1.3 mmol; see step (v) above (compound (b))) in DMF (18 mL) under nitrogen was added H-Aze-Pab(Teoc)×HCl (0.76 g, 1.7 mmol), PyBOP (0.75 g, 1.4 mmol), and DIPEA (0.43 g, 3.3 mmol) at 0° C. The reaction was stirred at 0° C. for 2 h and then at room temperature for 20 h. The mixture was concentrated in vacuo and the residue chromatographed twice on silica gel, eluting first with CHCl₃:EtOH (10:1), and then with EtOAc:EtOH (10:1) to afford the sub-title compound (0.69 g, 84%) as a crushable white foam.

mp: 108-118° C.

R_{f}=0.48 (10:1 CHCl₃:EtOH)

¹H NMR (300 MHz, CD₃OD, complex mixture of rotamers) Λ 7.78-7.81 (m, 2H), 7.40-7.43 (m, 2H), 7.09-7.12 (m, 1H), 6.96-7.02 (m, 2H), 6.16 (t, J=57 Hz, 1H), 5.09-5.20 and 4.75-4.80 (m, 2H), 3.95-4.55 (m, 8H), 2.10-2.75 (m, 2H), 1.04-1.11 (m, 2H), 0.07 (s, 9H)

APCI-MS: (M+1)=625 m/z

### (vii) Ph(3-Cl)(5-OCH₂CHF₂)—(R)CH(OH)C(O)-Aze-Pab×TFA

Ph(3-Cl)(5-OCH₂CHF₂)—(R)CH(OH)C(O)-Aze-Pab(Teoc) (0.086 g, 0.138 mmol; see step (vi) above), was dissolved in 3 mL of TFA and allowed to react for 1 h. TFA was evaporated and the residue was freeze dried from water/acetonitrile to yield 0.080 g (98%) of the title compound as its TFA salt.

¹H-NMR (300 MHz; CD₃OD) rotamers: δ 7.8-7.7 (m, 2H), 7.6-7.5 (m, 2H), 7.15-6.95 (m, 3H), 6.35-5.95 (m, 1H), 5.20 (m, 1H, minor rotamer), 5.14 (s, 1H, major rotamer), 5.10 (s, 1H, minor rotamer), 4.80 (m, 1H, major rotamer), 4.6-4.0 (m, 6H), 2.70 (m, 1H, minor rotamer), 2.53 (m, 1H, major rotamer), 2.29 (m, 1H, major rotamer), 2.15 (m, 1H, minor rotamer).

¹³C-NMR (100 MHz; CD₃OD): (carbonyl and/or amidine carbons, rotamers) δ 174.0, 173.8, 173.4, 172.9, 168.2

MS (m/z) 481.2 (M+1)⁺

### EXAMPLE 18

### Ph(3-Cl)(5-OCH₂CHF₂)—(R)CH(OH)C(O)-Aze-Pab(OMe)

To a solution of Ph(3-Cl)(5-OCH₂CHF₂)—(R)CH(OH)C(O)OH (0.30 g, 1.7 mmol; see Example 17(v) above (compound (b))) in DMF (15 mL) under nitrogen was added H-Aze-Pab(OMe)×2HCl (0.49 g, 1.5 mmol), PyBOP (0.65 g, 1.2 mmol), and DIPEA (0.36 g, 2.8 mmol) at 0° C. The reaction was stirred at 0° C. for 2 h and then at room temperature for 20 h. The mixture was concentrated in vacuo and the residue chromatographed three times on silica gel, eluting first with CHCl₃:EtOH (10:1), then with EtOAc:EtOH (10:1), and finally with CHCl₃:MeOH (20:1) to afford the title compound (0.47 g, 81%) as a crushable white foam.

mp: 65-75° C.

R_{f}=0.37 (10:1 CHCl₃:EtOH)

¹H NMR (300 MHz, CD₃OD, complex mixture of rotamers) A 7.58-7.60 (m, 2H), 7.32-7.35 (m, 2H), 7.09-7.12 (m, 1H), 6.96-7.02 (m, 2H), 6.16 (t, J=55 Hz, 1H), 5.08-5.18 and 4.74-4.80 (m, 2H), 3.96-4.50 (m, 6H), 3.80 (s, 3H), 2.10-2.75 (m, 2H)

APCI-MS: (M+1)=511 m/z.

### EXAMPLE 19

### Ph(3-Cl)(5-OCH₂F)—(R)CH(OH)C(O)-Aze-Pab×TFA

### (i) Ph(3-Cl)(5-TMSO)—(R,S)CH(OTMS)CN

To a solution of 3-chloro-5-hydroxybenzaldehyde (9.8 g, 62.6 mmol; see Example 1(ii) above) and ZnI₂ (5.0 g, 15.7 mmol) in anhydrous CH₂Cl₂ (500 mL) at 0° C. was added trimethylsilyl cyanide (13.7 g, 138 mmol). The reaction mixture was allowed to warm to room temperature and stirred overnight. Water (250 mL) was added, and the layers were separated. The aqueous layer was extracted with CH₂Cl₂ (2×300 mL). The combined organic extracts were dried (Na₂SO₄), filtered, and concentrated in vacuo to afford the sub-title compound (16.9 g, 83%) as a yellow oil that was used without further purification.

R_{f}=0.42 (3:1 Hex:EtOAc)

¹H NMR (300 MHz, CDCl₃) δ 7.06 (s, 1H), 6.86 (s, 2H), 5.40 (s, 1H), 0.30 (s, 9 H), 0.24 (s, 9 H).

### (ii) Ph(3-Cl)(5-OH)—(R,S)CH(OH)C(O)OH

A solution of Ph(3-Cl)(5-OTMS)—(R,S)CH(OTMS)CN (22.6 g, 68.8 mmol; see step (i) above) in concentrated HCl (200 mL) was refluxed under nitrogen for 3 h. The reaction was cooled to 0° C. and basified slowly with 2N NaOH. The mixture was washed with Et₂O (3×100 mL) to remove the organic impurities. The aqueous layer was acidified with 2N HCl and extracted with EtOAc (3×200 mL). The combined organic extracts were dried (Na₂SO₄), filtered, and concentrated in vacuo to afford the sub-title compound (9.3 g, 67%) as a brown oil that was used without further purification.

R_{f}=0.23 (6:3:1 CHCl₃:MeOH:concentrated NH₄OH)

¹H NMR (300 MHz, CD₃OD) δ 7.05 (s, 1H), 6.94 (s, 1H), 6.73 (s, 1H), 5.03(s, 1H).

### (iii) Ph(3-Cl)(5-OH)—(R,S)CH(OH)C(O)OEt

To a solution of Ph(3-Cl)(5-OH)—(R,S)CH(OH)C(O)OH (9.3 g, 46.0 mmol; see step (ii) above) in absolute EtOH (200 mL) was added concentrated sulfuric acid (0.25 mL) and the reaction was refluxed under nitrogen for 4 h. The reaction was cooled to 0° C. and solid NaHCO₃ (0.2 g) was added. The reaction was concentrated in vacuo and partitioned with saturated NaHCO₃ (100 mL) and Et₂O (3×50 mL). The combined organic extracts were dried (Na₂SO₄), filtered, and concentrated in vacuo to give the sub-title compound (6.9 g, 65%) as a yellow oil which was used without further purification.

R_{f}=0.62 (6:3:1 CHCl₃:MeOH:concentrated NH₄OH).

¹H NMR (300 MHz, CDCl₃) δ 6.99 (s, 1H), 6.81 (s, 2H), 5.07 (s, 1H), 4.16-4.32 (m, 2H), 1.23 (t, J=7 Hz, 3H).

### (iv) Ph(3-Cl)(5-OCH₂F)—(R,S)CH(OH)C(O)OEt

To a solution of Ph(3-Cl)(5-OH)—(R,S)CH(OH)C(O)OEt (6.1 g, 26.8 mmol; see step (iii) above) in DMF (100 mL) in a sealed flask under nitrogen at 0° C. was added cesium carbonate (13.1 g, 40.2 mmol). The reaction mixture was stirred at 0° C. for 15 minutes followed by the addition of potassium iodide (0.5 g, 2.7 mmol). The reaction was cooled to −78° C. and chlorofluoromethane (18.4 g, 268 mmol) was bubbled into the vessel. The sealed flask was then allowed to warm to room temperature and stirred for 18 h. The reaction mixture was cooled to 0° C., vented carefully to remove any excess chlorofluoromethane, and partitioned with H₂O (20 mL) and Et₂O (3×50 mL). The combined organics were washed with brine (2×50 mL), dried (Na₂SO₄), filtered and concentrated in vacuo. Flash chromatography on silica gel eluting with Hex:EtOAc (gradient-from 9:1 to 3:1) afforded the sub-title compound (2.4 g, 35%) as a light yellow oil. Note: The compound is faintly uv-visible on TLC. It can be visualised by staining the TLC with bromocresol green.

R_{f}=0.46 (2:1 Hex:EtOAc)

¹H NMR (300 MHz, CDCl₃) δ 7.21 (s, 1H), 7.08 (s, 1H), 7.05 (s, 1H), 5.70 (d, J_{H-F}=54 Hz, 2H), 5.12 (d, J=5 Hz, 1H), 3.80-4.35 (m, 2H), 3.50 (d, J=5 Hz, 1H), 1.26 (t, J=7 Hz, 3H).

### (v) Ph(3-Cl)(5-OCH₂F)—(R,S)CH(OH)C(O)OH

To a solution of Ph(3-Cl)(5-OCH₂F)—(R,S)CH(OH)C(O)OEt (1.8 g, 6.8 mmol; see step (iv) above) in H₂O: THF (30 mL, 1:2) at 0° C. under nitrogen was added lithium hydroxide monohydrate (0.40 g, 10.3 mmol). The mixture was stirred at 0° C. for 2 h. The reaction mixture was concentrated in vacuo and partitioned with H₂O (5 mL) and Et₂O (2×20 mL). The aqueous layer was acidified carefully with 0.2N HCl at 0° C. and extracted with EtOAc (3×30 mL). The combined organics were dried (Na₂SO₄), filtered and concentrated in vacuo to afford the sub-title compound (1.4 g, 87%) as a colourless oil which solidified to a white solid upon standing.

R_{f}=0.43 (6:2:1 CHCl₃:MeOH:Et₃N)

¹H NMR (300 MHz, CD₃OD) δ 7.24 (s, 1H), 7.17 (s, 1H), 7.07 (s, 1H), 5.78 (d, J_{H-F}=54 Hz, 2H), 5.13 (s, 1H).

### (vi) Ph(3-Cl)(5-OCH₂F)—(R)CH(OH)C(O)OH (a) and Ph(3-Cl)(5-OCH₂F)—(S)CH(OAc)C(O)OH (b)

A mixture of Ph(3-Cl)(5-OCH₂F)—(R,S)CH(OH)C(O)OH (3.2. g, 13.9 mmol; see step (v) above) and Lipase PS “Amano” (1.9 g) in vinyl acetate (150 mL) and MTBE (150 mL) was heated at 70° C. under a nitrogen atmosphere for 3 d. The reaction mixture was cooled, filtered through Celite® and the filter cake washed with EtOAc. The filtrate was concentrated in vacuo and subjected to flash chromatography on a silica gel eluting with CHCl₃:MeOH:Et₃N (15:1:0.5) to afford the triethylamine salt of the sub-title compound (a) (0.50 g, 21%) that was used without neutralisation. In addition, the triethylamine salt of the sub-title compound (b) (0.46 g, 20%) was obtained.

Data for Sub-Title Compound (a):

R_{f}=0.19 (15:1:0.5 CHCl₃:MeOH:Et₃N)

¹H NMR (300 MHz, CD₃OD) δ 7.26 (s, 1H), 7.18 (s, 1H), 6.97 (s, 1H), 5.74 (d, J_{H-F}=54 Hz, 2H), 4.81 (s, 1H), 3.17 (q, J=7 Hz, 6H), 1.28 (t, J=7 Hz, 9H).

Data for Sub-Title Compound (b)

R_{f}=0.33 (15:1:0.5 CHCl₃:MeOH:Et₃N)

¹H NMR (300 MHz, CD₃OD) δ 7.28 (s, 1H), 7.19 (s, 1H), 7.09 (s, 1H), 5.76 (d, J_{H-F}=54 Hz, 2H), 5.75 (s, 1H), 3.17 (q, J=7 Hz, 6H), 2.16 (s, 3H), 1.28 (t, J=7 Hz, 9H).

### (vii) Ph(3-Cl)(5-OCH₂F)—(R)CH(OH)C(O)-Aze-Pab(Teoc)

To a solution of the triethylamine salt of Ph(3-Cl)(5-OCH₂F)—(R)CH(OH)C(O)OH (0.50 g, 1.50 mmol; see step (vi) above) and HAze-Pab(Teoc).HCl (0.87 g, 1.90 mmol) in dry DMF (15 mL) under nitrogen at 0° C. was added PyBOP (0.85 g, 2.60 mmol) and DIPEA (0.48 g, 3.70 mmol). The reaction was allowed to warm to room temperature and stirred overnight. The reaction mixture was concentrated in vacuo and flash chromatographed twice on silica gel, eluting first with CHCl₃:EtOH (9:1) and second with EtOAc:EtOH (20:1) to afford the sub-title compound (0.23 g, 26%) as a crushable white foam.

Mp: 88-92° C.

R_{f}=0.61 (9:1 CHCl₃:EtOH)

¹H NMR (300 MHz, CD₃OD, complex mixture of rotamers) δ 7.81 (d, J=8 Hz, 2H), 7.40-7.42 (m, 2H), 7.06-7.23 (m, 3H), 5.76 (d, J_{H-F}=51 Hz, 2H), 5.10-5.16 and 4.77-4.83 (m, 2H), 3.80-4.49 (m, 6H), 2.30-2.53 (m, 2H), 1.08 (t, J=7 Hz, 2H), 0.08(s, 9H).

APCI-MS (M+1)=593 m/z

### (viii) Ph(3-Cl)(5-OCH₂F)—(R)CH(OH)C(O)-Aze-Pab×TFA

Ph(3-Cl)(5-OCH₂F)—(R)CH(OH)C(O)-Aze-Pab(Teoc) (0.051 g, 0.086 mmol; see step (vii) above), was dissolved in 3 mL of TFA and allowed to react for 20 min. TFA was evaporated and the residue was freeze dried from water/acetonitrile. The product was 95% pure with 5% of defluoromethylated material. Attempts to purify it by preparative RPLC with CH₃CN:0.1M NH₄OAc failed, and the material, partially as an acetate, was dissolved in 5 mL of TFA, evaporated and freeze dried to yield 26 mg (51%) of the title compound as its TFA salt. Purity: 95%.

¹H-NMR (600 MHz; CD₃OD) rotamers: δ 7.8-7.7 (m, 2H), 7.6-7.5 (m, 2H), 7.21 (s, 1H, major rotamer), 7.17 (s, 1H, minor rotamer), 7.13 (s, 1H, major rotamer), 7.09 (s, 1H, minor rotamer), 7.07 (m, 1H, major rotamer), 7.04 (m, 1H, minor rotamer), 5.73 (d, 2H), 5.18 (m, 1H, minor rotamer), 5.16 (s, 1H, major rotamer), 5.09 (s, 1H, minor rotamer), 4.78 (m, 1H, minor rotamer), 4.56 (d, 1H, major rotamer), 4.50 (d, 1H, minor rotamer), 4.46 (d, 1H, minor rotamer), 4.45 (d, 1H, major rotamer), 4.35 (m, 1H, major rotamer), 4.14 (m, 1H, major rotamer), 4.05 (m, 1H, minor rotamer), 3.97 (m, 1H, minor rotamer), 2.68 (m, 1H, minor rotamer), 2.52 (m, 1H, major rotamer), 2.28 (m, 1H, major rotamer), 2.19 (m, 1H, minor rotamer).

¹³C-NMR (150 MHz; CD₃OD): (carbonyl and/or amidine carbons, rotamers) δ 173.9, 173.3, 172.9, 168.2.

ESI-MS+: (M+1)=449 (m/z)

### EXAMPLE 20

### Ph(3-Cl)(5-OCH₂F)—(R)CH(OH)C(O)-Aze-Pab(OMe)

To a solution of the triethylamine salt of Ph(3-Cl)(5-OCH₂F)—(R)CH(OH)C(O)OH (0.60 g, 1.80 mmol; see Example 19(vi)) and HAze-Pab(OMe).2HCl (0.79 g, 2.30 mmol) in DMF (15 mL) under nitrogen at 0° C. was added PyBOP (1.04 g, 1.90 mmol) and DIPEA (0.58 g, 4.50 mmol). The reaction was allowed to warm to room temperature and stirred overnight. The reaction mixture was concentrated in vacuo and flash chromatographed three times on silica gel, eluting first with CHCl₃:EtOH (9:1) and then twice with EtOAc:EtOH (20:1) to afford the title compound (0.22 g, 26%) as a crushable white foam.

Mp: 66-70° C.

R_{f}=0.45 (9:1 CHCl₃:EtOH)

¹H NMR (300 MHz, CD₃OD, complex mixture of rotamers) δ 7.59 (d, J=8 Hz, 2H), 7.32 (d, J=7 Hz, 2H), 7.06-7.23 (m, 3H), 5.75 (s, J_{H-F}=54 Hz, 1H), 5.10-5.16 and 4.78-4.84 (m, 2H), 4.11-4.45 (m, 4H), 3.80 (s, 3H), 2.10-2.75 (m, 2H).

¹³C-NMR (150 MHz; CD₃OD): (carbonyl and/or amidine carbons, rotamers) δ 173.0, 170.8, 170.7, 152.5.

APCI-MS:(M+1)=479 m/z

### EXAMPLE 21

### Ph(3-Cl)(5-OCH₂CH₂F)—(R)CH(OH)C(O)-Aze-Pab×TFA

### (i) (2-Monofluoroethyl) methanesulfonate

To a magnetically stirred solution of 2-fluoroethanol (5.0 g, 78.0 mmol) in CH₂Cl₂ (90 mL) under nitrogen at 0° C. was added triethylamine (23.7 g, 234 mmol) and methanesulfonyl chloride (10.7 g, 93.7 mmol). The mixture was stirred at 0° C. for 1.5 h, diluted with CH₂Cl₂ (100 mL) and washed with 2N HCl (100 mL). The aqueous layer was extracted with CH₂Cl₂ (50 mL) and the combined organic extracts washed with brine (75 mL), dried (Na₂SO₄), filtered and concentrated in vacuo to afford the sub-title compound (9.7 g, 88%) as a yellow oil which was used without further purification.

¹H NMR (300 MHz, CDCl₃) δ 4.76 (t, J=4 Hz, 1H), 4.64 (t, J=4 Hz, 1H), 4.52 (t, J =4 Hz, 1H), 4.43 (t, J=4 Hz, 1H), 3.09 (s, 3H).

### (ii) 3-Chloro-5-monofluoroethoxybenzaldehyde

To a solution of 3-chloro-5-hydroxybenzaldehyde (8.2 g, 52.5 mmol; see Example 1(ii) above) and potassium carbonate (9.4 g, 68.2 mmol) in DMF (10 mL) under nitrogen was added a solution of (2-monofluoroethyl) methanesulfonate (9.7 g, 68.2 mmol; see step (i) above) in DMF (120 mL) dropwise at room temperature. The mixture was heated to 100° C. for 5 h and then stirred overnight at room temperature. The reaction was cooled to 0° C., poured into ice-cold 2N HCl and extracted with EtOAc. The combined organic extracts were washed with brine, dried (Na₂SO₄), filtered and concentrated in vacuo. The brown oil was chromatographed on silica gel eluting with Hex:EtOAc (4:1) to afford the sub-title compound (7.6 g, 71%) as a yellow oil.

¹H NMR (300 MHz, CDCl₃) δ 9.92 (s, 1H), 7.48 (s, 1H), 7.32 (s, 1H), 7.21 (s, 1H), 4.87 (t, J=4 Hz, 1H), 4.71 (t, J=3 Hz, 1H), 4.33 (t, J=3 Hz, 1H), 4.24 (t, J=3 Hz, 1H).

### (iii) Ph(3-Cl)(5-OCH₂CH2F)—(R,S)CH(OTMS)CN

To a solution of 3-chloro-5-monofluoroethoxybenzaldehyde (7.6 g, 37.5 mmol; see step (ii) above) and zinc iodide (3.0 g, 9.38 mmol) in CH₂Cl₂ (310 mL) was added trimethylsilyl cyanide (7.4 g, 75.0 mmol) dropwise at 0° C. under nitrogen. The mixture was stirred at 0° C. for 3 h and at room temperature overnight. The reaction was diluted with H₂O (300 mL), the organic layer was separated, dried (Na₂SO₄), filtered and concentrated in vacuo to afford the sub-title compound (10.6 g, 94%) as a brown oil that was used without further purification or characterisation.

### (iv) Ph(3-Cl)(5-OCH₂CH₂F)—(R,S)CH(OH)C(O)OH

Concentrated hydrochloric acid (100 mL) was added to Ph(3-Cl)(5-OCH₂CH₂F)—(R,S)CH(OTMS)CN (10.6 g, 5.8 mmol; see step (iii) above) and the solution stirred at 100° C. for 3 h. After cooling to room temperature, the reaction was further cooled to 0° C., basified slowly with 3N NaOH (˜300 mL) and washed with Et₂O (3×200 mL). The aqueous layer was acidified with 2N HCl (80 mL) and extracted with EtOAc (3×300 mL). The combined EtOAc extracts were dried (Na₂SO₄), filtered and concentrated in vacuo to afford the sub-title compound (8.6 g, 98%) as a pale yellow solid that was used without further purification.

R_{f}=0.28 (90:8:2 CHCl₃:MeOH:concentrated NH₄OH)

¹H NMR (300 MHz, CD₃OD) δ 7.09 (s, 1H), 7.02 (s, 1H), 6.93 (s, 1H), 5.11 (s, 1H), 4.77-4.81 (m, 1H), 4.62-4.65 (m, 1H), 4.25-4.28 (m, 1H), 4.15-4.18 (m, 1H).

### (v) Ph(3-Cl)(5-OCH₂CH₂F)—(S)CH(OAc)C(O)OH (a) and Ph(3-C1)(5-OCH₂CH₂F)—(R)CH(OH)C(O)OH (b)

A solution of Ph(3-Cl)(5-OCH₂CH₂F)—(R,S)CH(OH)C(O)OH (8.6 g, 34.5 mmol; see step (iv) above) and Lipase PS “Amano” (4.0 g) in vinyl acetate (250 mL) and MTBE (250 mL) was heated at 70° C. under nitrogen for 3 d. The reaction was cooled to room temperature and the enzyme removed by filtration through Celite®. The filter cake was washed with EtOAc and the filtrate concentrated in vacuo. Chromatography on silica gel eluting with CHCl₃:MeOH:Et₃N (90:8:2) afforded the triethylamine salt of sub-title compound (a) as a yellow oil. In addition, the triethylamine salt of sub-title compound (b) (4.0 g) was obtained. The salt of sub-title compound (b) was dissolved in H₂O (250 mL), acidified with 2N HCl and extracted with EtOAc (3×200 mL). The combined organic extracts were dried (Na₂SO₄), filtered and concentrated in vacuo to yield the sub-title compound (b) (2.8 g, 32%) as a yellow oil.

Data for Sub-Title Compound (b):

R_{f}=0.28 (90:8:2 CHCl₃:MeOH:concentrated NH₄OH)

¹H NMR (300 MHz, CD₃OD) δ 7.09 (s, 1H), 7.02 (s, 1H), 6.93 (s, 1H), 5.11 (s, 1H), 4.77-4.81 (m, 1H), 4.62-4.65 (m, 1H), 4.25-4.28 (m, 1 H), 4.15-4.18 (m, 1H).

### (vi) Ph(3-Cl)(5-OCH₂CH₂F)—(R)CH(OH)C(O)-Aze-Pab(Teoc)

To a solution of Ph(3-Cl)(5-OCH₂CH₂F)—(R)CH(OH)C(O)OH (940 mg, 3.78 mmol; see step (v) above) in DMF (30 mL) under nitrogen at 0° C. was added HAze-Pab(Teoc).HCl (2.21 g, 4.91 mmol), PyBOP (2.16 g, 4.15 mmol), and DIPEA (1.22 g, 9.45 mmol). The reaction was stirred at 0° C. for 2 h and then at room temperature for 4 h. The mixture was concentrated in vacuo and the residue chromatographed twice on silica gel, eluting first with CHCl₃:EtOH (15:1) and second with EtOAc:EtOH (20:1) to afford the sub-title compound (450 mg, 20%) as a crushable white foam.

Mp: 80-88° C.

R_{f}=0.60 (10:1 CHCl₃:EtOH)

¹H NMR (300 MHz, CD₃OD, complex mixture of rotamers) δ 7.79 (d, J=8 Hz, 2H), 7.42 (d, J=8 Hz, 2H), 7.05-7.08 (m, 1H), 6.93-6.99 (m, 2H), 5.08-5.13 (m, 1H), 4.75-4.80 (m, 2H), 4.60-4.68 (m, 1H), 3.95-4.55 (m, 8H), 2.10-2.75 (m, 2H), 1.05-1.11 (m, 2H), 0.08 (s, 9H).

APCI-MS: (M+1)=607 m/z.

### (vii) Ph(3-Cl)(5-OCH₂CH₂F)—(R)CH(OH)C(O)-Aze-Pab×TFA

Ph(3-Cl)(5-OCH₂CH₂F)—(R)CH(OH)C(O)-Aze-Pab(Teoc) (0.357 g, 0.589 mmol; see step (vi) above), was dissolved in 10 mL of TFA and allowed to react for 40 min. TFA was evaporated and the residue was freeze dried from water/acetonitrile to yield 0.33 g (93%) of the title compound as its TFA salt.

¹H-NMR (600 MHz; CD₃OD) rotamers: δ 7.8-7.7 (m, 2H), 7.54 (d, 2H), 7.08 (s, 1H, major rotamer), 7.04 (s, 1H, minor rotamer), 6.99 (s, 1H, major rotamer), 6.95 (s, 1H), 6.92 (s, 1H, minor rotamer), 5.18 (m, 1H, minor rotamer), 5.14 (s, 1H, major rotamer), 5.08 (s, 1H, minor rotamer), 4.80 (m, 1H, major rotamer), 4.73 (m, 1H), 4.65 (m, 1H), 4.6-4.4 (m, 2H), 4.35 (m, 1H, major rotamer), 4.21 (doublet of multiplets, 2H), 4.12 (m, 1H, major rotamer), 4.06 (m, 1H, minor rotamer), 3.99 (m, 1H, minor rotamer), 2.69 (m, 1H, minor rotamer), 2.53 (m, 1H, major rotamer), 2.29 (m, 1H, major rotamer), 2.14 (m, 1H, minor rotamer).

¹³C-NMR (150 MHz; CD₃OD): (carbonyl and/or amidine carbons) δ 172.8, 172.1, 167.4.

ESI-MS+: (M+1)=463 (m/z)

### EXAMPLE 22

### Ph(3-Cl)(5-OCH₂CH₂F)—(R)CH(OH)C(O)-Aze-Pab(OMe)

To a solution of Ph(3-Cl)(5-OCH₂CH₂F)—(R)CH(OH)C(O)OH (818 mg, 3.29 mmol; see Example 21(v) above) in DMF (30 mL) under nitrogen at 0° C. was added HAze-Pab(OMe)-2HCl (1.43 g, 4.27 mmol), PyBOP (1.89 g, 3.68 mmol), and DIPEA (1.06 g, 8.23 mmol). The reaction was stirred at 0° C. for 2 h and then at room temperature overnight. The mixture was concentrated in vacuo and the residue chromatographed two times on silica gel, eluting first with CHCl₃:EtOH (15:1) and second with EtOAc:EtOH (20:1) to afford the title compound (880 mg, 54%) as a crushable white foam.

Mp: 65-72° C.

R_{f}=0.60 (10:1 CHCl₃:EtOH)

¹H NMR (300 MHz, CD₃OD, complex mixture of rotamers) δ 7.58-7.60 (d, J=8 Hz, 2H), 7.34 (d, J=7 Hz, 2H), 7.05-7.08 (m, 2H), 6.95-6.99 (m, 1H), 5.08-5.13 (m, 1H), 4.77-4.82 (m, 1H), 4.60-4.68 (m, 1H), 3.99-4.51 (m, 7H), 3.82 (s, 3H), 2.10-2.75 (m, 2H).

¹³C-NMR (150 MHz; CD₃OD): (carbonyl and/or amidine carbons) δ 173.3, 170.8, 152.5.

APCI-MS: (M+1)=493 m/z.

### EXAMPLE 23

### Ph(3-Cl)(5-OCH(CH₂F)₂)—(R)CH(OH)C(O)-Aze-Pab×TFA

### (i) 1,3-Difluoroisopropyl methanesulfonate

To a magnetically stirred solution of 1,3-difluoro-2-propanol (7.0 g, 72.8 mmol) in CH₂Cl₂ (100 mL) under nitrogen at 0° C. was added triethylamine (22.1 g, 219 mmol) and methanesulfonyl chloride (10.0 g, 87.4 mmol). The mixture was stirred at 0° C. for 3 h. The mixture was washed with 2N HCl (150 mL) and the layers were separated. The aqueous layer was extracted with CH₂Cl₂ (200 mL) and the combined organic extracts washed with brine (100 mL), dried (Na₂SO₄), filtered and concentrated in vacuo to afford the sub-title compound (11.5 g, 91%) as a yellow oil which was used without further purification.

¹H NMR (300 MHz, CDCl₃) δ 4.97-5.08 (m, 1H), 4.75-4.77 (m, 2H), 4.59-4.61 (m, 2H), 3.12(s,3H).

### (ii) Ph(3-Cl)(5-OCH(CH₂F)₂)CHO

To a solution of 3-chloro-5-hydroxybenzaldehyde (8.0 g, 50.7 mmol; see Example 1(ii) above) and potassium carbonate (9.1 g, 66.0 mmol) in DMF (75 mL) under nitrogen was added a solution of 1,3-difluoroisopropyl methanesulfonate (11.5 g, 66.0 mmol; see step (i) above) in DMF (75 mL) dropwise at room temperature. The mixture was heated to 110° C. for 18 h. The reaction was cooled to 0° C., poured into ice-cold 2N HCl (200 mL) and extracted with EtOAc (3×250 mL). The combined organic extracts were dried (Na₂SO₄), filtered and concentrated in vacuo. The brown oil was chromatographed on silica gel eluting with Hex:EtOAc (4:1) to afford the sub-title compound (4.4 g, 37%) as a yellow oil.

¹H NMR (300 MHz, CDCl₃) δ 9.92 (s, 1H), 7.51 (s, 1H), 7.36 (s, 1H), 7.26 (s, 1H), 4.70-4.89 (m, 3H), 4.63-4.68 (m, 2H).

### (iii) Ph(3-Cl)(5-OCH(CH₂F)₂)—(R,S)CH(OTMS)CN

To a solution of Ph(3-Cl)(5-OCH(CH₂F)₂)CHO (4.4 g, 18.7 mmol; see step (ii) above) and zinc iodide (1.5 g, 4.67 mmol) in CH₂Cl₂ (200 mL) at 0° C. under nitrogen was added trimethylsilyl cyanide (3.7 g, 37.3 mmol) dropwise. The mixture was stirred at 0° C. for 3 h and overnight at room temperature, then diluted with H₂O (200 mL). The organic layer was separated, dried (Na₂SO₄), filtered and concentrated in vacuo to afford the sub-title compound (5.5 g, 87%) as a brown oil that was used without further purification.

¹H NMR (300 MHz, CDCl₃) δ 7.12 (s, 1H), 7.00 (s, 2H), 5.42 (s, 1H), 4.70-4.80 (m, 3H), 4.59-4.64 (m, 2H), 0.26 (s, 9H).

### (iv) Ph(3-Cl)(5-OCH(CH₂F)₂)-(R,S)CH(OH)C(O)OH

Concentrated hydrochloric acid (50 mL) was added to Ph(3-Cl)(5-OCH(CH₂F)₂)-(R,S)CH(OTMS)CN (5.5 g, 16.3 mmol; see step (iii) above) and the solution stirred at 100° C. for 1.5 h. After cooling to room temperature, the reaction was further cooled to 0° C., basified slowly with 3N NaOH (˜200 mL) and washed with Et₂O (3×200 mL). The aqueous layer was acidified with 2N HCl (75 mL) and extracted with EtOAc (3×200 mL). The combined EtOAc extracts were dried (Na₂SO₄), filtered and concentrated in vacuo to afford the sub-title compound (4.6 g, 100%) as a brown oil that was used without further purification.

¹H NMR (300 MHz, CD₃OD) δ 7.14 (s, 1H), 7.08 (s, 1H), 7.02 (s, 1H), 5.12 (s, 1H), 4.70-4.90 (m, 3H), 4.52-4.67 (m, 2H).

### (v) Ph(3-Cl)(5-OCH(CH₂F)₂)—(S)CH(OAc)C(O)OH (a) and Ph(3-Cl)(5-OCH(CH₂F)₂)-(R)CH(OH)C(O)OH (b)

A solution of Ph(3-Cl)(5-OCH(CH₂F)₂)—(R,S)CH(OH)C(O)OH (4.6 g, 16.4 mmol; see step (iv) above) and Lipase PS “Amano” (3.0 g) in vinyl acetate (150 mL) and MTBE (150 mL) was heated at 70° C. under nitrogen for 2.5 d. The reaction was cooled to room temperature, the enzyme removed by filtration through Celite®. The filter cake was washed with EtOAc and the filtrate concentrated in vacuo. Chromatography on silica gel eluting with CHCl₃:MeOH:Et₃N (90:8:2) afforded the triethylamine salt of the sub-title compound (a) as a yellow oil. In addition, the triethylamine salt of the sub-title compound (b) (2.2 g) was obtained and the salt was dissolved in H₂O (100 mL), acidified with 2N HCl and extracted with EtOAc (3×200 mL). The combined organic extracts were dried (Na₂SO₄), filtered and concentrated in vacuo to yield the sub-title compound (b) (1.4 g, 29%) as a yellow oil.

Data for Sub-Title Compound (b):

¹H NMR (300 MHz, CD₃OD) δ 7.14 (s, 1H), 7.08 (s, 1H), 7.02 (s, 1H), 5.12 (s, 1H), 4.70-4.90 (m, 3H), 4.52-4.67 (m, 2H).

### (vi) Ph(3-Cl)(5-OCH(CH₂F)₂)—(R)CH(OH)C(O)-Aze-Pab(Teoc)

To a solution of Ph(3-Cl)(5-OCH(CH₂F)₂)—(R)CH(OH)C(O)OH (824 mg, 2.94 mmol; see step (v) above) in DMF (30 mL) under nitrogen at 0° C. was added HAze-Pab(Teoc).HCl (1.71 g, 3.81 mmol), PyBOP (1.68 g, 3.23 mmol), and DIPEA (949 mg, 7.34 mmol). The reaction was stirred at 0° C. for 2 h and then at room temperature overnight. The mixture was concentrated in vacuo and the residue chromatographed twice on silica gel, eluting first with CHCl₃:EtOH (15:1), and second with EtOAc:EtOH (20:1) to afford the sub-title compound (720 mg, 38%) as a crushable white foam.

Mp: 78-84° C.

R_{f}=0.62(10:1 CHCl₃:EtOH)

¹H NMR (300 MHz, CD₃OD, complex mixture of rotamers) δ 7.79 (d, J=8 Hz, 2H), 7.42 (d, J=8 Hz, 2H), 7.00-7.12 (m, 3H), 5.08-5.20 (m, 1H), 3.97-4.80 (m, 12H), 2.10-2.75 (m, 2H), 1.05-1.11 (m, 2H), 0.08 (s, 9H).

APCI-MS: (M+1)=639 m/z.

### (vii) Ph(3-Cl)(5-OCH(CH₂F)₂)—(R)CH(OH)C(O)-Aze-Pab×TFA

Ph(3-Cl)(5-OCH(CH₂F)₂)—(R)CH(OH)C(O)-Aze-Pab(Teoc) (0.129 g, 0.202 mmol; see step (vi) above), was dissolved in 3 mL of TFA and allowed to react for 20 min. TFA was evaporated and the residue was freeze dried from water/acetonitrile to yield 0.123 g (100%) of the title compound as its TFA salt.

¹H-NMR (400 MHz; CD₃OD) rotamers: δ 7.8-7.7 (m, 2H), 7.55 (d, 2H), 7.2-7.0 (m, 3H), 5.18 (m, 1H, minor rotamer), 5.15 (s, 1H, major rotamer), 5.08 (s, 1H, minor rotamer), 4.80 (m, 1H, major rotamer partly obscured by the CD₃OH peak), 4.75-4.4 (m, 7H), 4.38 (m, 1H, major rotamer), 4.15 (m, 1H, major rotamer), 4.1-3.9 (m, 2H, 2 signals from minor rotamer), 2.70 (m, 1H, minor rotamer), 2.53 (m, 1H, major rotamer), 2.30 (m, 1H, major rotamer), 2.15 (m, 1H, minor rotamer).

¹³C-NMR (100 MHz; CD₃OD): (carbonyl and/or amidine carbons, rotamers) δ 172.9, 172.6, 172.2, 171.7, 167.1.

ESI-MS+: (M+1)=495 (m/z)

### EXAMPLE 24

### Ph(3-Cl)(5-OCH(CH₂F)₂)—(R)CH(OH)C(O)-Aze-Pab(OMe)

To a solution of Ph(3-Cl)(5-OCH(CH₂F)₂)—(R)CH(OH)C(O)OH (513 mg, 1.83 mmol; see Example 23(v) above) in DMF (30 mL) under nitrogen at 0° C. was added HAze-Pab(OMe).2HCl (797 mg, 2.38 mmol), PyBOP (1.04 g, 2.01 mmol), and DIPEA (591 mg, 4.57 mmol). The reaction was stirred at 0° C. for 2 h and then at room temperature overnight. The mixture was concentrated in vacuo and the residue chromatographed two times on silica gel, eluting first with CHCl₃:EtOH (15:1) and second with EtOAc:EtOH (20:1) to afford the title compound (370 mg, 39%) as a crushable white foam.

Mp: 58-63° C.

R_{f}=0.66 (10:1 CHCl₃:EtOH)

¹H NMR (300 MHz, CD₃OD, complex mixture of rotamers) δ 7.58-7.60 (d, J=8 Hz, 2H), 7.34 (d, J=8 Hz, 2H), 7.00-7.12 (m, 3H), 5.08-5.20 (m, 1H), 4.65-4.82 (m, 3H), 4.28-4.65 (m, 5H), 3.92-4.18 (m, 2H), 3.82 (s, 3H), 2.10-2.75 (m, 2H).

¹³C-NMR (150 MHz; CD₃OD): (carbonyl and/or amidine carbons) δ 173.2, 170.8, 152.5.

APCI-MS: (M+1)=525 m/z.

### EXAMPLE 25

### Ph(3-F)(5-OCHF₂)—(R)CH(OH)C(O)-Aze-Pab×TFA

### (i) 1-Bromo-3-fluoro-5-benzyloxybenzene

Sodium hydride (60% dispersion in oil, 24.0 g, 0.48 mol) was added portionwise to a stirred solution of anhydrous benzyl alcohol (64.5 g, 0.60 mol) in THF (1.0 L). After the mixture was stirred for 1 h, a solution of 1-bromo-3,5-difluorobenzene (76.8 g, 0.40 mmol) in THF (100 mL) was added dropwise over a period of 1 h. The reaction was stirred at room temperature for 2 d. Water (400 mL) was added and the THF was removed in vacuo. The aqueous layer was extracted with hexane (3×150 mL). The combined organic extracts were washed with 2N NaOH (2×100 mL) then, dried (Na₂SO₄), filtered and concentrated in vacuo to afford the sub-title compound (110.7 g, 98%) as a light yellow oil that was used without further purification.

R_{f}=0.47 (Hex)

¹H NMR (300 MHz, CDCl₃) δ 7.36-7.41 (m, 5H), 6.94 (bs, 1H), 6.87 (d, J_{H-F}=8 Hz, 1H), 6.63 (d, J_{H-F}=10 Hz, 1H), 5.03 (s, 2H).

### (ii) 3-Bromo-5-fluorophenol

To a solution of 1-bromo-3-fluoro-5-benzyloxybenzene (110.0 g, 0.39 mol; see step (i) above) and N,N-dimethylaniline (474.0 g, 3.92 mol) in anhydrous CH₂Cl₂ (1.0 L) at 0° C. was added aluminium chloride (156.0 g, 1.17 mol). After 10 min, the ice bath was removed and the stirring was continued for 2 h. The reaction was quenched by the cautious addition of 3N HCl (600 mL). The layers were separated, and the aqueous layer was extracted with CH₂Cl₂ (2×150 mL). The combined organic extracts were washed with 2N HCl (250 mL) and H₂O (3×250 mL). To the organic layer was added 15% KOH (500 mL), and the layers were separated. The organic layer was further extracted with 2 N KOH (2×70 mL). The combined aqueous layers were washed with CH₂Cl₂ (3×100 mL) and then acidified with 4N HCl. The aqueous layer was extracted with Et₂O (3×125 mL) then, the combined Et₂O extracts were dried (Na₂SO₄), filtered and concentrated in vacuo to afford the sub-title compound (69.0 g, 92%) as a brown oil that was used without further purification.

Mp: 33-35° C.

R_{f=}0.25 (CHCl₃)

¹H NMR (300 MHz, DMSO-d₆) δ 10.38 (s, 1H), 6.90 (dd, J_{H-F}=11 Hz, J=2 Hz, 1H), 6.81 (s, 1H), 6.59 (dt, J_{H-F}=11 Hz, J=2 Hz, 1H).

APCI-MS: (M−1)=189 m/z

### (iii) 1-Bromo-3-fluoro-5-difluoromethoxybenzene

A mixture of 3-bromo-5-fluorophenol (6.1 g, 31.0 mmol; see step (ii) above) and chlorodifluoromethane (13.0 g, 150.0 mmol) in i-PrOH (100 mL) and 30% KOH (80 mL) was heated in a sealed flask for 18 h at 80-85° C. The reaction mixture was cooled to room temperature and the layers were separated. The organic layer was concentrated in vacuo to afford a colourless oil. The aqueous layer was extracted with Et₂O (3×30 mL). The crude oil and the combined organic extracts were washed with 2N NaOH (3×30 mL) and H₂O (3×30 mL). The organics were then dried (Na₂SO₄), filtered through a small silica gel plug, and concentrated in vacuo to afford the sub-title compound (6.1 g, 79%) as a colourless oil that was used without further purification.

¹H NMR (300 MHz, CDCl₃) δ 7.11-7.14 (m, 2H), 6.84 (dt, J=9 Hz, J=2 Hz, 1H), 6.50 (t,

J_{H-F}=72 Hz, 1H).

### (iv) 1-Fluoro-3-difluoromethoxy-5-vinylbenzene

Tri(butyl)vinylstannane (7.0 g, 22.2 mmol) was added to a suspension of 1-bromo-3-fluoro-5-difluoromethoxybenzene (4.9 g, 20.2 mmol; see step (iii) above), dichlorobis(triphenylphosphine)palladium(II) (1.42 g, 2.02 mmol) and anhydrous lithium chloride (0.90 g, 20.2 mmol) in THF (40 mL) under nitrogen at 65° C. and the mixture was stirred for 5 h. The reaction mixture was cooled to 0° C. and 1N NaOH (90 mL) was added. The biphasic mixture was vigorously stirred for 1 h then the layers were separated. The aqueous layer was extracted with Et₂O (3×70 mL). The combined organic layers were washed with 2N NaOH (2×40 mL) and H₂O (40 mL) then dried (Na₂SO₄), filtered and concentrated in vacuo. Flash chromatography on silica gel eluting with hexane afforded the sub-title compound (2.2 g, 57%) as a colourless oil.

R_{f=}0.47 (Hex)

¹H NMR (300 MHz, CDCl₃) δ 6.93-6.99 (m, 2H), 6.73-6.78 (m, 1H), 6.67 (dd, J=18 Hz, J=11 Hz, 1H), 6.51 (t, J_{H-F}=73 Hz, 1H), 5.77 (d, J=18 Hz, 1H), 5.36 (d, J=11 Hz, 1H).

### (v) Ph(3-F)(5-OCHF₂)—(R)CH(OH)CH₂OH

2-Methyl-2-propanol (140 mL), H₂O (140 mL), and AD-mix-β (39.2 g) were combined together and cooled to 0° C. 1-Fluoro-3-difluoromethoxy-5-vinylbenzene (5.0 g, 26.4 mmol; see step (iv) above) dissolved in a small amount of 2-methyl-2-propanol was added at once, and the heterogeneous slurry was vigorously stirred at 0° C. until TLC revealed the absence of the starting material. The reaction was quenched at 0° C. by addition of sodium sulfite (42.0 g) and then warmed to room temperature and stirred for 60 min. The reaction mixture was extracted with Et₂O (3×120 mL). The combined organic extracts were dried (Na₂SO₄), filtered and concentrated in vacuo. Flash chromatography on silica gel eluting with CHCl₃:EtOAc (3:2) afforded the sub-title compound (5.8 g, 98%) as a colourless oil.

R_{f=}0.41 (3:2 CHCl₃:EtOAc)

¹H NMR (300 MHz, CDCl₃) δ 6.96-6.99 (m, 2H), 6.77-6.82 (m, 1H), 6.51 (t, J_{H-F}=73 Hz, 1H), 4.79-4.85 (m, 1H), 3.76-3.84 (m, 1H), 3.58-3.66 (m, 1H), 2.66 (d, J=3 Hz, 1H), 2.00 (t, J=6 Hz, 1H).

HPLC Analysis: 89.2%, >99% ee, ChiralPak AD Column (95:5 Hex:EtOH mobile phase).

### (vi) Ph(3-F)(5-OCHF₂)-(R)CH(OH)CH₂OTBS

A solution of Ph(3-F)(5-OCHF₂)—(R)CH(OH)CH₂OH (5.5 g, 24.7 mmol; see step (v) above), 4-(dimethylamino)pyridine (121 mg, 1.0 mmol) and triethylamine (3.0 g, 29.6 mmol) in anhydrous CH₂Cl₂ (100 mL) was cooled to 0° C. A 1.0 M solution of tert-butyldimethylsilyl chloride in CH₂Cl₂ (26.0 mL, 26.0 mmol) was added dropwise, and the reaction mixture was allowed to warm to room temperature and stirred overnight. Saturated ammonium chloride solution (60 mL) was added, and the layers were separated. The organic layer was washed with saturated ammonium chloride solution (60 mL) and H₂O (2×35 mL) then dried (Na₂SO₄), filtered, and concentrated in vacuo. Flash chromatography on silica gel eluting with CHCl₃:Hex (3:1) afforded the sub-title compound (7.9 g, 85%) as a yellow oil.

R_{f=}0.47 (3:1 CHCl₃:Hex)

¹H NMR (300 MHz, CDCl₃) δ 6.95-6.98 (m, 2H), 6.76-6.79 (m, 1H), 6.51 (t, J_{H-F}=73 Hz, 1H), 4.71-4.74 (m, 1H), 3.75-3.80 (m, 1H), 3.48-3.54 (m, 1H), 2.99 (bs, 1H), 0.91 (s, 9H), 0.05 (s, 3H), 0.00 (s, 3H).

### (vii) Ph(3-F)(5-OCHF₂)—(R)CH(OMEM)CH₂OTBS

To a solution of Ph(3-F)(5-OCHF₂)—(R)CH(OH)CH₂OTBS (7.9 g, 0.51 mmol; see step (vi) above) and DIPEA (4.9 g, 48.1 mmol) in anhydrous CH₂Cl₂ (50 mL) at 0° C. under nitrogen was added dropwise 2-methoxyethoxymethyl chloride (6.6 g, 48.1 mmol). The mixture was stirred for 24 h. Saturated ammonium chloride solution (70 mL) was added, and the layers were separated. The organic layer was washed with saturated ammonium chloride solution (70 mL) and H₂O (3×60 mL) then dried (Na₂SO₄), filtered and concentrated in vacuo to afford the sub-title compound (8.8 g, 99%) as a yellow oil that was used without further purification.

R_{f=}0.41 (4:1 CHCl₃:EtOAc)

¹H NMR (300 MHz, CDCl₃) δ 7.20 (s, 1H), 7.06 (s, 1H), 7.02 (s, 1H), 6.50 (t, J_{H-F}=73 Hz, 1H), 4.79-4.81 (m, 1H), 4.66-4.68 (m, 2H), 3.47-3.82 (m, 6H), 3.36 (s, 3H), 0.85 (s, 9H), 0.01 (s, 3H), 0.00 (s, 3H).

### (viii) Ph(3-F)(5-OCHF₂)—(R)CH(OMEM)CH₂OH

To a solution of Ph(3-F)(5-OCHF₂)—(R)CH(OMEM)CH₂OTBS (9.3 g, 21.9 mmol; see step (vii) above) in THF (60 mL) at room temperature was added a 1.0 M solution of tetrabutylammonium fluoride in THF (70.0 mL, 70.0 mmol) and the mixture was stirred overnight under nitrogen. The reaction was concentrated in vacuo. The yellow residue was dissolved in Et₂O (100 mL) and hexane (100 mL) and washed successively with saturated ammonium chloride solution (2×150 mL) and H₂O (3×70 mL). The organic layer was dried (Na₂SO₄), filtered and concentrated in vacuo. Flash chromatography on silica gel eluting with Hex:EtOAc (1:1) afforded the sub-title compound (4.2 g, 62%) as a yellow oil.

R_{f=}0.42 (1:1 Hex:EtOAc)

¹H NMR (300 MHz, CDCl₃) δ 6.91-6.95 (m, 2H), 6.75-6.81 (m, 1H), 6.51 (t, J_{H-F}=73 Hz, 1H), 4.80-4.82 (m, 1H), 4.70-4.74 (m, 2H), 3.88-3.93 (m, 1H), 3.67-3.71 (m, 3H), 3.53-3.56 (m, 2H), 3.39 (s, 3H), 2.96-2.99 (m, 1H).

### (ix) Ph(3-F)(5-OCHF₂)—(R)CH(OMEM)C(O)OH

A solution of Ph(3-F)(5-OCHF₂)—(R)CH(OMEM)CH₂OH (4.2 g, 13.4 mmol; see step (viii) above) in acetone (100 mL) was added to an aqueous 5% NaHCO₃ solution (35 mL). This magnetically stirred heterogeneous mixture was cooled to o0° C. and potassium bromide (159 mg, 1.3 mmol) and 2,2,6,6-tetramethyl-1-piperidinyloxy, free radical (2.2 g, 14.1 mmol) were added. Sodium hypochlorite (5.25%, 30 mL) was then added dropwise over a period of 20 min while the mixture was vigorously stirred and maintained at 0° C. After 1 h, additional sodium hypochlorite (30 mL) and 5% NaHCO₃ solution (35 mL) were added and stirring was continued at 0° C. for 2 h. The acetone was removed in vacuo. The aqueous layer was washed with Et₂O (4×40 mL). The aqueous layer was acidified to pH 3.5 with 10% citric acid and extracted with EtOAc (4×50 mL). The combined EtOAc extracts were successively washed with H₂O (4×30 mL) and brine (60 mL) then, dried (Na₂SO₄), filtered and concentrated in vacuo to afford the sub-title compound (4.3 g, 98%) as a colourless oil which was used without further purification.

R_{f=}0.74 (8.0:1.5:0.5 CHCl₃:MeOH:Et₃N)

¹H NMR (300 MHz, acetone-d₆) δ 7.16-7.18 (m, 2H), 7.16 (t, J_{H-F}=89 Hz, 1H), 7.00-7.03 (m, 1H), 5.30 (s, 1H), 4.88 (d, J=7 Hz, 1H), 4.80 (d, J=7 Hz, 1H), 3.54-3.75 (m, 2H), 3.46-3.49 (m, 2H), 3.28 (s, 3H).

### (x) Ph(3-F)(5-OCHF₂)—(R)CH(OMEM)C(O)-Aze-Pab(Teoc)

To a solution of Ph(3-F)(5-OCHF₂)—(R)CH(OMEM)C(O)OH (1.1 g, 3.4 mmol; see step (ix) above) in DMF (20 mL) under nitrogen at 0° C. was added HAze-Pab(Teoc).HCl (2.0 g, 4.4 mmol), PyBOP (1.9 g, 3.7 mmol), and DIPEA (1.1 g, 8.4 mmol). The reaction was stirred at 0° C. for 2 h and then at room temperature overnight. The mixture was concentrated in vacuo and the residue chromatographed twice on silica gel, eluting first with CHCl₃:EtOH (15:1) and second with EtOAc:EtOH (20:1) to afford the sub-title compound (1.3 g, 56%) as a crushable white foam.

R_{f=}0.65 (15:1 CHCl₃:EtOH)

¹H NMR (300 MHz, CD₃OD, complex mixture of rotamers) δ 7.80-7.84 (m, 2H), 7.40-7.46 (m, 2H), 6.95-7.16 (m, 3H), 6.92 and 6.88 (t, J_{H-F}=73 Hz, 1H), 5.28 and 5.08 (s, 1H), 5.18-5.22 and 4.70-4.78 (m, 1H), 4.50-4.75 (m, 1H), 4.30-4.49 (m, 2H), 4.21-4.26 (m, 3H), 3.97-4.08 (m, 1H), 3.35-3.72 (m, 6H), 3.30 (s, 3H), 2.10-2.75 (m, 2H), 1.05-1.11 (m, 2H), 0.08 (s, 9H).

### (xi) Ph(3-F)(5-OCHF₂)—(R)CH(OH)C(O)-Aze-Pab(Teoc)

A mixture of Ph(3-F)(5-OCHF₂)—(R)CH(OMEM)C(O)-Aze-Pab(Teoc) (590 mg, 0.87 mmol; see step (x) above) and carbon tetrabromide (287 mg, 0.87 mmol) in 2-propanol (20 mL) was refluxed for 1.5 h. The mixture was concentrated in vacuo then, partitioned with H₂O (50 mL) and EtOAc (3×50 mL). The aqueous layer was extracted with additional EtOAc (2×10 mL). The combined organic extracts were washed with brine (30 mL) then dried (Na₂SO₄), filtered and concentrated in vacuo. Flash chromatography on silica gel eluting with CHCl₃:EtOH (15:1) afforded the sub-title compound (60 mg, 12%) as a crushable white foam.

R_{f}=0.46 (15:1 CHCl₃:EtOH)

¹H NMR (300 MHz, CD₃OD, complex mixture of rotamers) δ 7.74 (d, J=8 Hz, 2H), 7.35-7.37 (m, 2H), 6.97-7.07 (m, 2H), 6.80-6.84 (m, 1H), 6.82 and 6.80 (t, J_{H-F}=73 Hz, 1H), 5.10 and 5.06 (s, 1H), 4.68-4.70 (m, 1H), 3.97-4.60 (m, 6H), 2.10-2.75 (m, 2H), 1.05-1.11 (m, 2H), 0.08 (s, 9H).

APCI-MS: (M+1) =595 m/z

### (xii) Ph(3-F)(5-OCHF₂)—(R)CH(OH)C(O)-Aze-Pab×TFA

Ph(3-F)(5-OCHF₂)—(R)CH(OH)C(O)-Aze-Pab(Teoc) (0.053 g, 0.089 mmol; see step (xi) above), was dissolved in 3 mL of TFA and allowed to react for 80 min while cooled on an ice bath. TFA was evaporated and the residue was freeze dried from water/acetonitrile to yield 0.042 g (80%) of the title compound as its TFA salt.

¹H-NMR (300 MHz; CD₃OD) rotamers: δ 7.7-7.6 (m, 2H), 7.5-7.4 (m, 2H), 7.1-6.6 (m, 4H), 5.2-5.0 (m, 1H plus minor rotamer of 1H), ca 4.8 (major rotamer of previous signal is obscured by the CD₃OH signal), 4.6-4.3 (m, 2H), 4.26 (m, 1H, major rotamer), 4.10 (m, 1H, major rotamer), 3.96 (m, 1H, minor rotamer), 3.89 (m, 1H, minor rotamer), 2.60 (m, 1H, minor rotamer), 2.44 (m, 1H, major rotamer), 2.19 (m, 1H, major rotamer), 2.05 (m, 1H, minor rotamer).

¹³C-NMR (100 MHz; CD₃OD): (carbonyl and/or amidine carbons) δ 172.8, 172.0, 167.0.

ESI-MS+: (M+1)=451 (m/z)

### EXAMPLE 26

### Ph(3-F)(5-OCHF₂)—(R)CH(OH)C(O)-Aze-Pab(OMe)

### (i) Ph(3-F)(5-OCHF₂)—(R)CH(OMEM)C(O)-Aze-Pab(OMe)

To a solution of Ph(3-F)(5-OCHF₂)—(R)CH(OMEM)C(O)OH (1.0 g, 3.1 mmol; see Example 25(ix) above) in DMF (30 mL) under nitrogen at 0° C. was added HAze-Pab(OMe)2.HCl (1.4 g, 4.1 mmol), PyBOP (1.8 g, 3.4 mmol), and DIPEA (1.0 g, 7.8 mmol). The reaction was stirred at 0° C. for 2 h and then at room temperature overnight. The mixture was concentrated in vacuo and the residue chromatographed two times on silica gel, eluting first with CHCl₃:EtOH (15:1) and second with EtOAc to afford the sub-title compound (1.5 g, 79%) as a crushable white foam.

R_{f}=0.24 (EtOAc)

¹H NMR (300 MHz, CD₃OD, complex mixture of rotamers) δ 7.58-7.62 (m, 2H), 7.32-7.38 (m, 2H), 7.03-7.16 (m, 3H), 6.92 and 6.88 (d, J_{H-F}=73 Hz, 1H), 5.27 and 5.08 (s, 1H), 5.22-5.15 and 4.75-4.80 (m, 1H), 4.38-4.65 (m, 5H), 3.92-4.27 (m, 1H), 3.82 (s, 3H), 3.43-3.68 (m, 4H), 3.29 (s, 3H), 2.28-2.85 (m, 2H).

### (ii) Ph(3-F)(5-OCHF₂)—(R)CH(OH)C(O)-Aze-Pab(OMe)

A mixture of Ph(3-F)(5-OCHF₂)—(R)CH(OMEM)C(O)-Aze-Pab(OMe) (828 mg, 2.33 mmol; see step (i) above) and carbon tetrabromide (525 mg, 2.33 mmol) in 2-propanol (20 mL) was refluxed for 8 h and then stirred at room temperature overnight. The mixture was concentrated in vacuo and the residue partitioned with H₂O (70 mL) and EtOAc (50 mL). The aqueous layer was extracted with EtOAc (2×25 mL). The combined organic extracts were washed with brine (35 mL) then, dried (Na₂SO₄), filtered and concentrated in vacuo. Flash chromatography on silica gel eluting with CHCl₃:EtOH (15:1) afforded the title compound (520 mg, 74%) as a crushable white foam.

Mp: 73-81° C.

R_{f}=0.43 (15:1 CHCl₃:EtOH)

¹H NMR (300 MHz, CD₃OD, complex mixture of rotamers) δ 7.59 (d, J=8 Hz, 2H), 7.32-7.37 (m, 2H), 7.05-7.14 (m, 2H), 6.87-6.92 (m, 1H), 6.90 and 6.86 (t, J_{H-F}=73 Hz, 1H), 5.13-5.18 and 4.75-4.85 (m, 2H), 4.15-4.45 (m, 4H), 3.81 (s, 3H), 2.10-2.75 (m, 2H).

¹³C-NMR (100 MHz; CD₃OD): (carbonyl and/or amidine carbons) δ 172.0, 171.4, 153.9.

APCI-MS: (M+1)=481 m/z

### EXAMPLE 27

### Ph(3-Br)(5-OCH₂F)—(R)CH(OH)C(O)-Aze-Pab×TFA

### (i) 1,3-Dibromo-5-benzyloxybenzene

Sodium hydride (9.9 g, 0.414 mol, 95% dry) was added in portions to a stirred solution of benzyl alcohol (41.0 g, 0.394 mol) in THF (1.0 L) at room temperature under a nitrogen atmosphere and stirred for 1 h. To this solution was added dropwise 1,3-dibromo-5-fluorobenzene (100.0 g, 0.394 mol). After stirring overnight, the mixture was partitioned with H₂O (600 mL) and EtOAc (4×600 mL). The combined organic extracts were dried (Na₂SO₄), filtered and concentrated in vacuo. Flash chromatography on silica gel eluting with hexanes afforded the sub-title compound (101.3 g, 75%) as a yellow oil.

¹H NMR (300 MHz, CDCl₃) δ 7.30-7.48 (m, 5H), 7.18 (s, 1H), 7.06 (s, 2H), 4.99 (s, 2H).

### (ii) 3,5-Dibromoyhenol

Aluminium chloride (11.7 g, 87.6 mmol) was added in portions to a solution of 1,3-dibromo-5-benzyloxybenzene (10.0 g, 29.2 mmol; see step (i) above) and N,N-dimethylaniline (35.4 g, 292 mmol) in CH₂Cl₂ (100 mL) at room temperature under a nitrogen atmosphere. After 30 min, the mixture was partitioned with 1N HCl (300 mL) and EtOAc (5×150 mL). The combined organic extracts were washed with saturated NaHCO₃ (150 mL) and brine (150 mL) then, dried (Na₂SO₄), filtered and concentrated in vacuo. Flash chromatography on silica gel eluting with Hex:EtOAc (9:1) afforded the sub-title compound (6.1 g, 82%) as a white solid.

¹H NMR (300 MHz, CDCl₃) δ 7.21 (s, 1H), 6.97 (s, 2H), 5.88 (bs, 1H).

### (iii) 1,3-Dibromo-5-monofluoromethoxybenzene

To a tared, sealed 350 mL round-bottomed pressure flask containing a suspension of 3,5-dibromophenol (10.0 g, 39.7 mmol; see step (ii) above) and Cs₂CO₃ (20.7 g, 63.5 mmol) in DMF (150 mL) at −78° C. was added chlorofluoromethane via bubbling for 5 min through the septum. The septum was replaced with a Teflon stopper and the flask was then sealed and allowed to warm to room temperature where the flask was weighed and determined to contain 9.0 g (131 mmol) of chlorofluoromethane. The solution was heated in an oil bath set at 70° C. overnight. The flask was cooled to room temperature, the pressure cautiously released and the contents diluted with water (100 mL). The aqueous layer was extracted with Et₂O (3×200 mL) then, the combined organics were dried (Na₂SO₄), filtered and concentrated in vacuo. Flash chromatography on silica gel eluting with hexanes afforded the sub-title compound (7.9 g, 71%) as a white solid.

¹H NMR (300 MHz, CDCl₃) δ 7.40 (s, 1H), 7.18 (s, 2H), 5.67 (d, J_{H-F}=53 Hz, 2H).

### (iv) 1-Bromo-3-monofluoromethoxy-5-vinylbenzene

Tri(butyl)vinyltin (10.0 g, 31.4 mmol) was added dropwise to a solution of 1,3-dibromo-5-monofluoromethoxybenzene (8.5 g, 29.9 mmol; see step (iii) above), tetrakis(triphenylphosphine)palladium(0) (690 mg, 0.599 mmol), and 2,6-di-tert-butyl-4-methylphenol (spatula tip) in toluene (100 mL) under nitrogen. The mixture was stirred at 70° C. for 8 h. The mixture was cooled to 0° C. and 1N NaOH (70 mL) was added. After 1 h, the mixture was extracted with CH₂Cl₂ (3×300 mL) then, the combined organics were dried (Na₂SO₄), filtered and concentrated in vacuo. Flash chromatography on silica gel eluting with hexanes afforded the sub-title compound (4.3 g, 57%) as a colourless oil.

¹H NMR (300 MHz, CDCl₃) δ 7.30 (s, 1H), 7.16 (s, 1H), 7.01 (s, 1H), 6.60 (dd, J=6 Hz, J=11 Hz, 1H), 5.74 (d, J=16 Hz, 1H), 5.67 (d, J_{H-F}=53 Hz, 2H), 5.32 (d, J=8 Hz, 1H).

### (v) Ph(3-Br)(5-OCH₂F)—(R)CH(OH)CH₂OH

2-Methyl-2-propanol (100 mL), H₂O (100 mL), and AD-mix-β (27.5 g) were combined together and cooled to 0° C. 1-Bromo-3-monofluoromethoxy-5-vinylbenzene (4.3 g, 17.3 mmol; see step (iv) above) was added at once, and the heterogeneous slurry was vigorously stirred at 0° C. until TLC revealed the absence of the starting material. The reaction was quenched at 0° C. by addition of saturated sodium sulfite (200 mL) and then warmed to room temperature and stirred for 60 min. The reaction mixture was extracted with EtOAc (3×150 mL). The combined organic extracts were dried (Na₂SO₄), filtered and concentrated in vacuo to afford the sub-title compound (4.9 g, 100%) as a colourless oil that was used without further purification.

¹H NMR (300 MHz, CD₃OD) δ 7.30 (s, 1H), 7.15 (s, 1H), 7.11 (s, 1H), 5.70 (d, J_{H-F}=53 Hz, 2H), 4.62-4.70 (m, 1H), 3.52-3.70 (m, 2H).

HPLC Analysis: 92.1%, 96.9% ee, ChiralPak AD Column (95:5 Hex:EtOH mobile phase).

### (vi) Ph(3-Br)(5-OCH₂F)—(R)CH(OMEM)CH₂OTBS

To a solution of Ph(3-Br)(5-OCH₂F)—(R)CH(OH)CH₂OH (4.9 g, 18.6 mmol; see step (v) above), 4-(dimethylamino)pyridine (453 mg, 3.71 mmol), and DIPEA (8.9 g, 93.0 mmol) in anhydrous CH₂Cl₂ (200 mL) was added dropwise a 1.0 M solution of tert-butyldimethylsilyl chloride in CH₂Cl₂ (22.3 mL, 22.3 mmol). The reaction mixture was stirred 10 h at room temperature. To the mixture was added DIPEA (8.9 g, 93.0 mmol) and 2-methoxyethoxymethyl chloride (13.9 g, 111 mmol) dropwise. After 16 h, additional 2-methoxyethoxymethyl chloride (2.2 g) was added and the reaction stirred overnight. The mixture was diluted with H₂O (100 mL), and the layers were separated. The aqueous layer was extracted with CH₂Cl₂ (3×200 mL) then, the combined organic layers were dried (Na₂SO₄), filtered and concentrated in vacuo. Flash chromatography on silica gel eluting with Hex:EtOAc (5:1) afforded the sub-title compound (4.8 g, 55%) as a colourless oil.

¹H NMR (300 MHz, CDCl₃) δ 7.29 (s, 1H), 7.22 (s, 1H), 7.05 (s, 1H), 5.74 (d, J_{H-F}=53 Hz, 2H), 4.84 (d, J=7 Hz, 1H), 4.70-4.74 (m, 2H), 3.50-3.91 (m, 6H), 3.42 (s, 3H), 0.90 (s, 9H), 0.05 (s, 3H), 0.01 (s, 3H).

### (vii) Ph(3-Br)(5-OCH₂F)—(R)CH(OMEM)CH₂OH

To a solution of Ph(3-Br)(5-OCH₂F)—(R)CH(OMEM)CH₂OTBS (4.7 g, 10.1 mmol; see step (vi) above) in THF (100 mL) was added a 1.0 M solution of tetrabutylammonium fluoride in THF (13.1 mL, 13.1 mmol) at room temperature and the mixture was stirred 1 h. The mixture was partitioned with H₂O (100 mL) and EtOAc (3×100 mL) then the combined organics were dried (Na₂SO₄), filtered and concentrated in vacuo to afford the sub-title compound (3.3 g, 92%) as a colourless oil that was used without further purification.

¹H NMR (300 MHz, CD₃OD) δ 7.22 (s, 1H), 7.14 (s, 1H), 7.03 (s, 1H), 5.71 (d, J_{H-F}=53 Hz, 2H), 4.80-4.82 (m, 1H), 4.58-4.66 (m, 2H), 3.71-3.77 (m, 1H), 3.39-3.65 (m, 5H), 3.27 (s, 3H).

### (viii) Ph(3-Br)(5-OCH₂F)—(R)CH(OMEM)C(O)OH

A solution of Ph(3-Br)(5-OCH₂F)—(R)CH(OMEM)CH₂OH (2.1 g, 6.0 mmol; see step (vii) above) in acetone (40 mL) was added to an aqueous 5% NaHCO₃ solution (15 mL). This magnetically stirred heterogeneous mixture was cooled to 0° C. and potassium bromide (70 mg, 0.60 mmol) and 2,2,6,6-tetramethyl-1-piperidinyloxy, free radical (976 mg, 5.8 mmol) were added. Sodium hypochlorite (5.25%, 15 mL) was then added dropwise over a period of 10 min while the mixture was vigorously stirred and maintained at 0° C. After 1 h, additional sodium hypochlorite (10 mL) and NaHCO₃ solution (20 mL) were added and stirring was continued at 0° C. for an additional 4 h. The acetone was removed on a rotary evaporator. The aqueous layer was diluted with 10% NaHCO₃ solution (30 mL) and was washed with Et₂O (3×20 mL). The aqueous layer was acidified to pH 3.5 with 10% citric acid and extracted with EtOAc (3×40 mL). The combined EtOAc extracts were washed with H₂O (3×50 mL) and brine (50 mL) then, dried (Na₂SO₄), filtered and concentrated in vacuo to afford the sub-title compound (1.7 g, 78%) as a colourless oil which was used without further purification.

¹H NMR (300 MHz, CD₃OD) δ 7.38 (s, 1H), 7.25 (s, 1H), 7.18 (s, 1H), 5.76 (d, J_{H-F}=53 Hz, 2H), 5.21 (s, 1H), 4.83 (d, J=7 Hz, 1H), 4.75 (d, J=7 Hz, 1H), 3.62-3.78 (m, 2H), 3.48-3.52 (m, 2H), 3.32 (s, 3H).

### (ix) Ph(3-Br)(5-OCH₂F)—(R)CH(OMEM)C(O)-Aze-Pab(Teoc)

To a solution of Ph(3-Br)(5-OCH₂F)—(R)CH(OMEM)C(O)OH (1.0 g, 2.72 mmol; see step (viii) above) in DMF (20 mL) under nitrogen at 0° C. was added HAze-Pab(Teoc).HCl (1.6 g, 3.5 mmol), PyBOP (1.6 g, 3.0 mmol), and DIPEA (880 mg, 6.81 mmol). The reaction was stirred at 0° C. for 2 h and then at room temperature overnight. The mixture was concentrated in vacuo and the residue chromatographed twice on silica gel, eluting first with CHCl₃:EtOH (15:1) and second with EtOAc:EtOH (20:1) to afford the sub-title compound (1.2 g, 62%) as a crushable white foam.

¹H NMR (300 MHz, CD₃OD, complex mixture of rotamers) δ 7.80-7.84 (m, 2H), 7.40-7.46 (m, 2H), 7.13-7.32 (m, 3H), 5.84-5.87 (m, 1H), 5.67-5.69 (m, 1H), 5.25 and 5.07 (s, 1H), 5.18-5.23 and 4.80-4.88 (m, 1H), 3.97-4.79 (m, 8H), 3.60-3.71 (m, 2H), 3.40-3.53 (m, 2H), 3.32 (s, 3H), 2.10-2.75 (m, 2H), 1.05-1.11 (m, 2H), 0.08 (s, 9H).

### (x) Ph(3-Br)(5-OCH₂F)—(R)CH(OH)C(O)-Aze-Pab(Teoc)

A mixture of Ph(3-Br)(5-OCH₂F)—(R)CH(OMEM)C(O)-Aze-Pab(Teoc) (347 mg, 0.478 mmol; see step (ix) above) and carbon tetrabromide (159 mg, 0.478 mmol) in 2-propanol (10 mL) was refluxed for 1.5 h. The mixture was concentrated in vacuo then partitioned with H₂O (20 mL) and EtOAc (3×30 mL). The combined organics were dried (Na₂SO₄), filtered and concentrated in vacuo. Flash chromatography on silica gel eluting with CHCl₃:EtOH (15:1) afforded the sub-title compound (59 mg, 19%) as a crushable white foam.

Mp: 81-87° C.

R_{f=}0.58 (9:1 CHCl₃:EtOH)

¹H NMR (300 MHz, CD₃OD, complex mixture of rotamers) δ 7.84 (d, J=8 Hz, 2H), 7.40-7.48 (m, 2H) 7.18-7.30 (m, 3H), 5.80 (d, J_{H-F}=53 Hz, 2H), 5.21 and 5.15 (s, 1H), 5.18-5.24 and 4.80-4.88 (m, 1H), 3.98-4.54 (m, 6H), 2.10-2.70 (m, 2H), 1.05-1.11 (m, 2H), 0.08 (s, 9H).

APCI-MS: (M+1)=637 m/z

### (xi) Ph(3-Br)(5-OCH₂F)—(R)CH(OH)C(O)-Aze-Pab×TFA

Ph(3-Br)(5-OCH₂F)—(R)CH(OH)C(O)-Aze-Pab(Teoc) (0.073 g, 0.11 mmol; see step (x) above), was dissolved in 5 mL of TFA and allowed to react for 90 min while being cooled on an ice bath. TFA was evaporated and the residue purified by prep RPLC with CH₃CN:0.1M NH₄OAc (30:70). The pertinent fractions were evaporated and freeze dried from water/acetonitrile to yield 49 mg (77%) of the title compound as its acetate salt.

¹H-NMR (300 MHz; CD₃OD) rotamers: δ 7.8-7.7 (m, 2H), 7.54 (m, 2H), 7.37 (s, 1H, major rotamer), 7.33 (s, 1H, minor rotamer), 7.25-7.1 (m, 2H), 5.75 (d, 2H), 5.22 (m, 1H, minor rotamer), 5.18 (s, 1H, major rotamer), 5.11 (s, 1H, minor rotamer), 4.80 (m, 1H, major rotamer), 4.6-4.4 (m, 2H), 4.37 (m, 1H, major rotamer), 4.16 (m, 1H, major rotamer), 4.1-3.9 (m, 2H, two signals from minor rotamer), 2.70 (m, 1H, minor rotamer), 2.52 (m, 1H, major rotamer), 2.30 (m, 1H, major rotamer), 2.15 (m, 1H, minor rotamer), 1.89 (s, 3H).

ESI-MS+: (M+1)=493/495 (m/z)

### EXAMPLE 28

### Ph(3-Br)(5-OCHF₂)—(R)CH(OH)C(O)-Aze-Pab×TFA

### (i) 1,3-Dibromo-5-difluoromethoxybenzene

To a tared, sealed 350 mL round-bottomed pressure flask containing a solution of 3,5-dibromophenol (10.0 g, 39.7 mmol; see Example 27(ii) above) in 2-propanol (100 mL) and 30% KOH (80 mL) at −78° C. was added chlorodifluoromethane via bubbling for 15 min through the septum. The septum was replaced with a Teflon stopper and the flask was then sealed and allowed to warm to room temperature where the flask was weighed and determined to contain 12.0 g (138 mmol) of chlorodifluoromethane. The solution was refluxed overnight in an oil bath set at 80° C. The flask was cooled to room temperature, the pressure cautiously released and the contents diluted with H₂O (200 mL). The aqueous layer was extracted with CHCl₃ (2×150 mL), then the combined organics were dried (Na2SO₄), filtered and concentrated in vacuo. The residue was purified by Kugelrohr distillation at 80° C. at 0.2 mm Hg to afford the sub-title compound (9.6 g, 80%) as clear liquid.

¹H NMR (300 MHz, CDCl₃) δ 7.55 (s, 1H), 7.26 (s, 2H), 6.52 (t, J_{H-F}=68 Hz, 1 H).

### (ii) 1-Bromo-3-difluoromethoxy-5-vinylbenzene

Tri(butyl)vinyltin (10.5 g, 33.1 mmol) was added dropwise to a solution of 1,3-dibromo-5-difluoromethoxybenzene (9.1 g, 30.1 mmol; see step (i) above), tetrakis(triphenylphosphine)palladium(0) (700 mg, 0.60 mmol), and 2,6-di-tert-butyl-4-methylphenol (spatula tip) in toluene (125 mL) under nitrogen. The mixture was stirred at 50° C. overnight. The mixture was cooled to 0° C. and 1N NaOH (70 mL) was added. After 1 h, the mixture was extracted with CH₂Cl₂ (3×300 mL) then, the combined organics were dried (Na₂SO₄), filtered and concentrated in vacuo. Flash chromatography on silica gel eluting with hexanes afforded the sub-title compound (5.1 g, 68%) as a colourless oil.

¹H NMR (300 MHz, CDCl₃) δ 7.53 (s, 1H), 7.18 (s, 1H), 7.08 (s, 1H), 6.60 (dd, J=6 Hz, J=11 Hz, 1H), 6.57 (t, J_{H-F}=68 Hz, 1H), 5.77 (d, J=11 Hz, 1H), 5.36 (d, J=8 Hz, 1H).

### (iii) Ph(3-Br)(5-OCHF₂)—(R)CH(OH)CH₂OH

2-Methyl-2-propanol (150 mL), H₂O (150 mL), and AD-mix-β (27.8 g) were combined together and cooled to 0° C. 1-Bromo-3-difluoromethoxy-5-vinylbenzene (4.6 g, 18.6 mmol; see step (ii) above) was added at once, and the heterogeneous slurry was vigorously stirred at 0° C. until TLC indicated the absence of the starting material, then the solution was warmed to room temperature and stirred overnight. The reaction was quenched at 0° C. by addition of saturated sodium sulfite (300 mL) and then warmed to room temperature and stirred for 60 min. The reaction mixture was extracted with EtOAc (3×200 mL). The combined organic extracts were dried (Na₂SO₄), filtered and concentrated in vacuo to afford the sub-title compound (5.0 g, 95%) as a colourless oil that was used without further purification.

¹H NMR (300 MHz, CD₃OD) δ 7.43 (s, 1H), 7.23 (s, 1H), 7.16 (s, 1H), 6.86 (t, J_{H-F}=75 Hz, 1H), 4.64-4.67 (m, 1H), 3.54-3.59 (m, 2H).

HPLC Analysis: 88.6%, 96.3% ee, ChiralPak AD Column (95:5 Hex:EtOH mobile phase).

### (iv) Ph(3-Br)(5-OCHF₂)—(R)CH(OMEM)CH₂OTBS

To a solution of Ph(3-Br)(5-OCHF₂)—(R)CH(OH)CH₂OH (4.9 g, 17.3 mmol; see step (iii) above), 4-(dimethylamino)pyridine (420 mg, 3.5 mmol), and DIPEA (11.2 g, 86.3 mmol) in anhydrous CH₂Cl₂ (250 mL) was added dropwise a 1.0 M solution of tert-butyldimethylsilyl chloride in CH₂Cl₂ (20.7 mL, 20.7 mmol). The reaction mixture was stirred overnight at room temperature. To the mixture was added DIPEA (11.2 g, 86.3 mmol) and 2-methoxyethoxymethyl chloride (12.9 g, 104 mmol) dropwise. After 3 d, additional 2-methoxyethoxymethyl chloride (3.3 g) was added and the reaction stirred overnight. The mixture was diluted with water (250 mL), and the layers were separated. The aqueous layer was extracted with CH₂Cl₂ (2×250 mL), then the combined organics were dried (Na₂SO₄), filtered and concentrated in vacuo. Flash chromatography on silica gel eluting with Hex:EtOAc (4:1) afforded the sub-title compound (4.3 g, 51%) as a colourless oil.

¹H NMR (300 MHz, CDCl₃) δ 7.40 (s, 1H), 7.25 (s, 1H), 7.08 (s, 1H), 6.58 (t, J_{H-F}=75 Hz, 1H), 4.84 (d, J=7 Hz, 1H), 4.70-4.74 (m, 2H), 3.50-3.91 (m, 6H), 3.42 (s, 3H), 0.90 (s, 9H), 0.12 (s, 3H), 0.05 (s, 3H).

### (v) Ph(3-Br)(5-OCHF₂)—(R)CH(OMEM)CH₂OH

To a solution of Ph(3-Br)(5-OCHF₂)—(R)CH(OMEM)CH₂OTBS (3.3 g, 6.9 mmol; see step (iv) above) in THF (60 mL) was added a 1.0 M solution of tetrabutylammonium fluoride in THF (9.0 mL, 9.0 mmol) at room temperature. The reaction was stirred for 45 min, then the mixture was partitioned with water (150 mL) and EtOAc (2×120 mL). The combined organics were dried (Na₂SO₄), filtered and concentrated in vacuo to afford the sub-title compound (2.5 g, 98%) as a yellow oil that was used without further purification.

¹H NMR (300 MHz, CD₃OD) δ 7.35 (s, 1H), 7.21 (s, 1H), 7.08 (s, 1H), 6.83 (t, J_{H-F}=73 Hz, 1H), 4.73 (d, J=7 Hz, 1H), 4.59-4.68 (m, 2H), 3.40-380 (m, 6H), 3.26 (s, 3H).

### (vi) Ph(3-Br)(5-OCHF₂)—(R)CH(OMEM)C(O)OH

A solution of Ph(3-Br)(5-OCHF₂)—(R)CH(OMEM)CH₂OH (3.0 g, 8.1 mmol; see step (v) above) in acetone (60 mL) was added to an aqueous 5% NaHCO₃ solution (25 mL). This magnetically stirred heterogeneous mixture was cooled to 0° C. then potassium bromide (100 mg, 0.81 mmol) and 2,2,6,6-tetramethyl-1-piperidinyloxy, free radical (1.3 g, 8.5 mmol) were added. Sodium hypochlorite (5.25%, 19 mL) was then added dropwise over a period of 10 min while the mixture was vigorously stirred and maintained at 0° C. After 1 h, additional sodium hypochlorite (17 mL) and NaHCO₃ solution (34 mL) were added and stirring was continued at 0° C. for an additional 4 h. The acetone was removed on a rotary evaporator. The aqueous layer was diluted with 10% NaHCO₃ solution (30 mL) and was washed with Et₂O (3×20 mL). The aqueous layer was acidified to pH 3.5 with 10% citric acid and extracted with EtOAc (3×40 mL). The combined EtOAc layers were washed with H₂O (3×50 mL) and brine (50 mL), and then dried (Na₂SO₄), filtered and concentrated in vacuo to afford the sub-title compound (2.1 g, 66%) as a colourless oil which was used without further purification.

¹H NMR (300 MHz, CD₃OD) δ 7.51 (s, 1H), 7.32 (s, 1H), 7.24 (s, 1H), 6.88 (t, J_{H-F}=73 Hz, 1H), 5.21 (s, 1H), 4.84 (d, J=7 Hz, 1H), 4.76 (d, J=7 Hz, 1H), 3.62-3.80 (m, 2H), 3.48-3.52 (m, 2H), 3.32 (s, 3H).

### (vii) Ph(3-Br)(5-OCHF₂)—(R)CH(OMEM)C(O)-Aze-Pab(Teoc)

To a solution of Ph(3-Br)(5-OCHF₂)—(R)CH(OMEM)C(O)OH (1.0 g, 2.62 mmol; see step (vi) above) in DMF (50 mL) under nitrogen at 0° C. was added HAze-Pab(Teoc).HCl (1.5 g, 3.38 mmol), PyBOP (1.5 g, 2.9 mmol), and DIPEA (840 mg, 6.50 mmol). The reaction was stirred at 0° C. for 2 h and then at room temperature overnight. The mixture was concentrated in vacuo and the residue chromatographed on silica gel eluting with CHCl₃:EtOH (15:1) to afford the sub-title compound (1.1 g, 59%) as a crushable white foam.

¹H NMR (300 MHz, CD₃OD, complex mixture of rotamers) δ 7.79-7.83 (m, 2H), 7.26-7.52 (m, 5H), 6.94 and 6.91 (t, J_{H-F}=73 Hz, 1H), 5.27 and 5.07 (s, 1H), 5.20-5.23 and 4.80-4.88 (m, 1H), 4.01-4.79 (m, 8H), 3.60-3.71 (m, 2H), 3.40-3.53 (m, 2H), 3.32 (s, 3H), 2.10-2.75 (m, 2H), 1.05-1.11 (m, 2H), 0.08 (s, 9H).

### (viii) Ph(3-Br)(5-OCHF₂)—(R)CH(OH)C(O)-Aze-Pab(Teoc)

A mixture of Ph(3-Br)(5-OCHF₂)—(R)CH(OMEM)C(O)-Aze-Pab(Teoc) (369 mg, 0.496 mmol; see step (vii) above) and carbon tetrabromide (165 mg, 0.496 mmol) in 2-propanol (10 mL) was refluxed for 12 h. The mixture was concentrated in vacuo, then partitioned with H₂O (15 mL) and EtOAc (5×20 mL). The combined organics were dried (Na₂SO₄), filtered and concentrated in vacuo. Flash chromatography on silica gel eluting with CHCl₃:EtOH (15:1) afforded the sub-title compound (134 mg, 41%) as a crushable white foam.

Mp: 92-98° C.

R_{f=}0.37 (9:1 CHCl₃:EtOH)

¹H NMR (300 MHz, CD₃OD, complex mixture of rotamers) δ 7.80-7.86 (m, 2H), 7.40-7.48 (m, 2H) 7.10-7.33 (m, 3H), 6.92 and 6.88 (t, J_{H-F}=73 Hz, 1H), 5.18 and 5.11 (s, 1H), 5.18-5.24 and 4.76-4.80 (m, 1H), 3.98-4.54 (m, 6H), 2.10-2.70 (m, 2H), 1.05-1.11 (m, 2H), 0.08 (s, 9H).

APCI-MS: (M+1)=655 m/z

### (ix) Ph(3-Br)(5-OCHF₂)—(R)CH(OH)C(O)-Aze-Pab×TFA

Ph(3-Br)(5-OCHF₂)—(R)CH(OH)C(O)-Aze-Pab(Teoc) (0.081 g, 0.124 mmol; see step (viii) above), was dissolved in 5 mL of TFA and allowed to react for 80 min while being cooled on an ice bath. TFA was evaporated and the residue purified by prep RPLC with CH₃CN:0.1M NH₄OAc (30:70). The pertinent fractions were evaporated and freeze dried from water/acetonitrile to yield 59 mg (83%) of the title compound as its acetate salt.

¹H-NMR (300 MHz; CD₃OD) rotamers: δ 7.8-7.7 (m, 2H), 7.6-7.4 (m, 3H), 7.3-7.2 (m, 2H), 6.89 (t, 1H, major rotamer), 6.87 (t, 1H, minor rotamer), 5.23 (m, 1H, minor rotamer), 5.21 (s, 1H, major rotamer), 5.13 (s, 1H, minor rotamer), 4.80 (m, 1H, major rotamer), 4.6-4.4 (m, 2H), 4.38 (m, 1H, major rotamer), 4.20 (m, 1H, major rotamer), 4.1-3.9 (m, 2H, two signals from minor rotamer), 2.70 (m, 1H, minor rotamer), 2.54 (m, 1H, major rotamer), 2.29 (m, 1H, major rotamer), 2.15 (m, 1H, minor rotamer), 1.89 (s, 3H).

¹³C-NMR (75 MHz; CD₃OD): (carbonyl and/or amidine carbons) δ 172.0, 171.7, 167.0.

MS (m/z) 511/513 (M+1)⁺

### EXAMPLE 29

### Ph(3-Br)(5-OCHF₂)—(R)CH(OH)C(O)-Aze-Pab(OMe)

### (i) Ph(3-Br)(5-OCHF₂)—(R)CH(OMEM)C(O)-Aze-Pab(OMe)

To a solution of Ph(3-Br)(5-OCHF₂)—(R)CH(OMEM)C(O)OH (957 mg, 2.48 mmol; see Example 28(vi) above) in DMF (30 mL) under nitrogen at 0° C. was added HAze-Pab(OMe)2.HCl (1.1 g, 3.2 mmol), PyBOP (1.4 g, 2.7 mmol), and DIPEA (804 mg, 6.2 mmol). The reaction was stirred at 0° C. for 2 h and then at room temperature overnight. The mixture was concentrated in vacuo and the residue chromatographed twice on silica gel, eluting first with CHCl₃:EtOH (9:1) and second with EtOAc:EtOH (15:1) to afford the sub-title compound (1.1 g, 72%) as a crushable white foam.

¹H NMR (300 MHz, CD₃OD, complex mixture of rotamers) δ 7.59-7.65 (m, 2H), 7.20-7.55 (m, 5H), 6.95 and 6.91 (t, J_{H-F}=73 Hz, 1H), 5.27 and 5.07 (s, 1H), 5.18-5.23 and 4.75-4.84 (m, 1H), 3.87-4.89 (m, 6H), 3.84 (s, 3H), 3.60-3.71 (m, 2H), 3.40-3.53 (m, 2H), 3.32 (s, 3H), 2.10-2.75 (m, 2H).

### (ii) Ph(3-Br)(5-OCHF₂)—(R)CH(OH)C(O)-Aze-Pab(OMe)

A mixture of Ph(3-Br)(5-OCHF₂)—(R)CH(OMEM)C(O)-Aze-Pab(OMe) (1.1 g, 1.8 mmol; see step (i) above) and carbon tetrabromide (583 mg, 1.8 mmol) in 2-propanol (30 mL) was refluxed for 2.5 d. During this time, additional carbon tetrabromide (5 portions of 50 mg at intervals for an additional 0.90 mmol) was added to ensure completion of the reaction. The mixture was concentrated in vacuo, then partitioned with H₂O (50 mL) and EtOAc (5×25 mL). The combined organics were dried (Na₂SO₄), filtered and concentrated in vacuo. Flash chromatography on silica gel eluting with CHCl₃:EtOH (15:1) afforded the title compound (460 mg, 50%) as a crushable white foam.

Mp: 71-75° C.

R_{f=}0.63 (9:1 CHCl₃:EtOH)

¹H NMR (300 MHz, CD₃OD, complex mixture of rotamers) δ 7.59 (d, J=8 Hz, 2H), 7.20-7.54 (m, 5H), 6.90 and 6.87 (t, J_{H-F}=73 Hz, 1H), 5.18 and 5.11 (s, 1H), 4.76-4.80 (m, 1H), 3.98-4.54 (m, 4H), 3.82 (s, 3H), 2.10-2.70 (m, 2H).

¹³C-NMR (100 MHz; CD₃OD): (carbonyl and/or amidine carbons, rotamers) δ 172.5, 172.1, 171.6, 154.1.

APCI-MS: (M+1)=542 m/z

### EXAMPLE 30

### Ph(3-Cl)(5-OCH₂CHF₂)—(R)CH(OH)C(O)-Aze-Pab(OH)

### (i) Ph(3-Cl)(5-OCH₂CHF₂)—(R)CH(OH)C(O)-Aze-Pab(Z)

Boc-Aze-Pab(Z) (see international patent application WO 97/02284, 92 mg, 0.197 mmol) was dissolved in 10 mL of EtOAc saturated with HCl(g) and allowed to react for 10 min. The solvent was evaporated and the residue was mixed with Ph(3-Cl)(5-OCH₂CHF₂)—(R)CH(OH)C(O)OH (50 mg, 0.188 mmol; see Example 17(v) above), PyBOP (109 mg, 0.209 mmol) and finally diisopropylethyl amine (96 mg, 0.75 mmol) in 2 mL of DMF. The mixture was stirred for 2 h and then poured into 50 mL of water and extracted three is times with EtOAc. The combined organic phase was washed with water, dried (Na₂SO₄) and evaporated. The crude product was flash chromatographed on silica gel with EtOAc:MeOH (9:1). Yield: 100 mg (87%).

¹H NMR (300 MHz, CD₃OD, mixture of rotamers) δ 7.85-7.75 (m, 2H), 7.45-7.25 (m, 7H), 7.11 (m, 1H, major rotamer), 7.08 (m, 1H, minor rotamer), 7.05-6.9 (m, 2H), 6.13 (bt, 1H), 5.25-5.05 (m, 3H), 4.77 (m, 1H, partially hidden by the CD₃OH signal), 4.5-3.9 (m, 7H), 2.64 (m, 1H, minor rotamer), 2.47 (m, 1H, major rotamer), 2.25 (m, 1H, major rotamer), 2.13 (m, 1H, minor rotamer)

### (ii) Ph(3-Cl)(5-OCH₂CHF₂)—(R)CH(OH)C(O)-Aze-Pab(OH)

Hydroxylamine hydrochloride (65 mg, 0.94 mmol) and triethylamine (0.319 g, 3.16 mmol) were mixed in 8 mL of THF and sonicated for 1 h at 40° C. Ph(3-Cl)(5-OCH₂CHF₂)—(R)CH(OH)C(O)-Aze-Pab(Z) (96 mg, 0.156 mmol; see step (i) above) was added with 8 mL more of THF. The mixture was stirred at 40° C. for 4.5 days. The solvent was evaporated and the crude product was purified by preparative RPLC with CH₃CN:0.1M NH₄OAc (40:60). Yield: 30 mg (38%). Purity: 99%.

¹H NMR (300 MHz, CD₃OD, mixture of rotamers) δ 7.6-7.55 (m, 2H), 7.35-7.3 (m, 2H), 7.12 (m, 1H, major rotamer), 7.09 (m, 1H, minor rotamer), 7.05-6.9 (m, 2H), 6.15 (triplet of multiplets, 1H), 5.15.(m, 1H, minor rotamer), 5.13 (s, 1H, major rotamer), 5.08 (s, 1H, minor rotamer), 4.77 (m, 1H, major rotamer), 4.5-4.2 (m, 5H), 4.08 (m, 1H, major rotamer), 3.97 (m, 1H, minor rotamer), 2.66 (m, 1H, minor rotamer), 2.50 (m, 1H major rotamer), 2.27 (m, 1H, major rotamer), 2.14 (m, 1H, minor rotamer).

¹³C-NMR (100 MHz; CD₃OD): (carbonyl and/or amidine carbons, mixture of rotamers) δ 172.8, 172.2, 171.4, 159.1, 158.9, 154.2.

APCI-MS: (M+1)=497/499 m/z

### EXAMPLE 31

### Ph(3-Cl)(5-OCH₂CH₂F)—(R)CH(OH)C(O)-Aze-Pab(OH)

### (i) Ph(3-Cl)(5-OCH₂CH₂F)—(R)CH(OH)C(O)-Aze-Pab(Z)

Boc-Aze-Pab(Z) (130 mg, 0.279 mmol) was dissolved in 15 mL of EtOAc saturated with HCl(g) and allowed to react for 10 min. The solvent was evaporated and the residue was mixed with Ph(3-Cl)(5-OCH₂CH₂F)—(R)CH(OH)—C(O)OH (63 mg, 0.188 mmol; see Example 21(v) above) in 3 mL of DMF, PyBOP (147 mg, 0.279 mmol) and finally diisopropylethyl amine (134 mg, 1.03 mmol). The mixture was stirred for 130 min and then poured into 75 mL of water and extracted three times with EtOAc. The combined organic phase was washed with water, dried (Na₂SO₄) and evaporated. The crude product was flash chromatographed on silica gel with EtOAc/MeOH =95/5. Yield: 119 mg (79%).

¹H NMR (400 MHz, CDCl₃) δ 8.06 (bt, 1H), 7.67 (d, 2H), 7.45-7.25 (m, 5H), 7.18 (d, 2H), 6.89 (m, 1H), 6.84 (m, 1H), 6.76 (m, 1H), 5.16 (s, 2H), 4.84 (s, 1H), 4.79 (m, 1H), 4.66 (doublet of multiplets, 2H), 4.4-4.3 (m, 2H), 4.10 (doublet of multiplets, 2H), 4.02 (m, 1H), 3.67 (m, 1H), 2.46 (m, 1H), 2.28 (m, 1H).

### (ii) Ph(3-Cl)(5-OCH₂CH₂F)—(R)CH(OH)C(O)-Aze-Pab(OH)

Hydroxylamine hydrochloride (80 mg, 1.16 mmol) and triethylamine (0.392 g, 3.87 mmol) were mixed in 9 mL of THF and sonicated for 1 h at 40° C. Ph(3-Cl)(5-OCH₂CH₂F)—(R)CH(OH)C(O)-Aze-Pab(Z) (96 mg, 0.156 mmol; see step (i) above) was added with 9 mL more of THF. The mixture was stirred at 40° C. for 48 h and 3 days at room temperature. The solvent was evaporated and the crude product was purified by preparative RPLC with CH₃CN:0.1M NH₄OAc (30:70). Yield: 72 mg (78%). Purity: 100%.

¹H NMR (400 MHz, CD₃OD, mixture of rotamers) δ 7.6-7.55 (m, 2H), 7.35-7.25 (m, 4H), 7.07 (m, 1H, major rotamer), 7.04 (m, 1H, minor rotamer), 7.0-6.9 (M, 2h), 5.12 (m, 1H, minor rotamer), 5.08 (s, 1H, minor rotamer), 5.04 (s, 1H), 4.78 (m, 1H, major rotamer), 4.68 (doublet of multiplets, 2 H), 4.5-4.25 (m, 3H), 4.20 (doublet of multiplets, 2H) 4.06 (m, 1H, major rotamer), 3.97 (m, 1H, minor rotamer), 2.65 (m, 1H, minor rotamer), 2.48 (m, 1H major rotamer), 2.27 (m, 1H, major rotamer), 2.14 (m, 1H, minor rotamer)

¹³C-NMR (100 MHz; CD₃OD): (carbonyl and/or amidine carbons, mixture of rotamers) δ 172.3, 171.5, 159.8, 154.3

APCI-MS: (M+1)=479/481 m/z

### EXAMPLE 32

### Ph(3-Cl)(5-OCHF₂)—(R)CH(OH)—C(O)-Pro-Pab

### (i) Boc-Pro-Pab(Teoc)

Boc-Pro-Pab(Z) (see international patent application WO 97/02284, 15.0 g, 0.0321 mol) was dissolved in 150 mL of ethanol and 200 mg 10% Pd/C (50% moisture) was added. The mixture was stirred and hydrogenated at atmospheric pressure for 2 h, filtered through Hyflo and concentrated. The product was used without further purification. Of this product was taken 10 g (0.029 mol), which was dissolved in 300 mL of THF. Teoc-p-nitrophenyl carbonate (10 g, 0.035 mol) was added. A solution of potassium carbonate (5.2 g, 0.038 mol) in 50 mL of water was added over 3 min and the resulting solution was stirred for 3 days, concentrated and the remainder was extracted with EtOAc three times. The combined organic layer was washed with water, dried (Na₂SO₄) and evaporated. The crude product was flash chromatographed on silica gel using methylene chloride:acetone (4:1). Yield: 9.8 g (69%).

### (ii) Ph(3-Cl)(5-OCHF₂)—(R)CH(OH)—C(O)-Pro-Pab(Teoc)

Boc-Pro-Pab(Teoc) (107 mg, 0.218 mmol; see step (i) above) was dissolved in 10 mL of EtOAc saturated with HCl(g) and allowed to react for 10 min. The solvent was evaporated and the residue was mixed with Ph(3-Cl)(5-OCHF₂)—(R)CH(OH)C(O)OH (50 mg, 0.198 mmol; see Example 1(viii) above) in 3 mL of DMF, PyBOP (115 mg, 0.218 mmol) and finally diisopropylethyl amine (104 mg, 0.80 mmol). The mixture was stirred for 2 h and then poured into 75 mL of water and extracted three times with EtOAc. The combined organic phase was washed with water, dried (Na₂SO₄) and evaporated. The crude product was flash chromatographed on silica gel with EtOAc:MeOH (95:5). Yield: 89 mg (72%).

¹H NMR (400 MHz, CDCl₃) δ 7.54 (bt, 1H), 7.47 (d, 2H), 7.12 (m, 1H), 7.08 (d, 2H), 7.02 (m, 1H), 6.95 (m, 1H), 6.50 (t, 1H), 5.21 (s, 1H), 4.42 (m, 1H), 4.35-4.15 (m, 3H), 3.59 (m, 1H), 2.94 (m, 1H), 2.1-1.7 (m, 4H), 1.06 (m, 2H), 0.04 (s, 9H).

### (iii) Ph(3-Cl)(5-OCHF₂)—(R)CH(OH)—C(O)-Pro-Pab×TFA

Ph(3-Cl)(5-OCHF₂)—(R)CH(OH)C(O)-Pro-Pab(Teoc) (85 mg, 0.136 mmol; see step (ii) above) was dissolved in 1 mL of methylene chloride and cooled on an ice bath. TFA (4 mL) was added and the reaction was stirred for 90 min. The TFA was evaporated and the residue was freeze-dried from water and acetonitrile. Yield: 79 mg (92%). Purity: 94%.

¹H NMR (400 MHz, CD₃OD, mixture of rotamers) δ 7.85-7.7 (m, 2H), 7.58 (d, 2H, major rotamer), 7.47 (d, 2H, minor rotamer), 7.35 (m, 1H, major rotamer), 7.27 (m, 1H, minor rotamer), 7.2.7.1 (m, 2H), 6.88 (t, 1H), 5.38 (s, 1H, major rotamer), 5.22 (s, 1H, minor rotamer), 4.58 (d, 1H), 4.5-4.2 (m, 2H), 3.8-3.5 (m, 1H), 3.35 (m, 1H), 2.2-1.8

¹³C-NMR (100 MHz; CD₃OD): (carbonyl and/or amidine carbons) δ 173.6, 171.1, 167.0.

APCI-MS: (M+1)=481/483 m/z

### EXAMPLE 33

### Ph(3-Cl)(5-OCHF₂)—(R)CH(OH)—C(O)-Pro-Pab(OMe)

### (i) 4-Azidomethyl-N-methoxy-benzamidine

4-Azidomethylbenzonitrile (17.3 g, 0.109 mol; Nishiyama et al; Chem. Lett. (1982) 1477) was dissolved in 500 mL of toluene and 200 mL of absolute ethanol. The solution was cooled to −10° C. and HCl(g) was bubbled through until saturation. The mixture was kept in the refrigerator for 2 days when most of the solvents were evaporated. Diethyl ether was added and was decanted off. The product was re-dissolved in a solution of O-methylhydroxyl amine (10.5 g, 0.125 mol) and triethyl amine (56 mL) in 200 mL of methanol. The mixture was allowed to stand for 3 days whence the methanol was evaporated with addition of EtOAc. The organic phase was washed with water, dilute HOAc and aqueous sodium bicarbonate, dried (Na₂SO₄) and diluted with more EtOAc to a total volume of 500 mL. A sample of 25 mL was evaporated to dryness. The remainder was 932 mg. Total yield: 18.6 g (83%).

### (ii) 4-Aminomethyl-N-methoxy-benzamidine

To a solution of 4-azidomethyl-N-methoxy-benzamidine (11.3 g, 0.055 mol; see step (i) above) in 200 mL of ethanol was added 200 mg of PtO₂. The mixture was hydrogenated with constant bubbling of hydrogen for 4 h and subsequently filtered through Celite® and evaporated. Yield: 7.34 g (74%).

### (iii) Boc-Pro-Pab(OMe)

To a suspension of Boc-Pro-OH (9.7 g, 0.045 mol), 4-aminomethyl-N-methoxy-benzamidine (7.34 g, 0.041 mol; see step (ii) above) and dimethylaminopyridine (7.8 g, 0.064 mol) in 300 mL of acetonitrile was added EDC base (11.7 mL, 0.068 mol). The mixture was stirred for 18 h, concentrated and partitioned between water and EtOAc. The organic layer was washed with water, aqueous sodium bicarbonate, dried (MgSO₄) and evaporated. The crude product was flash chromatographed on silica gel with EtOAc. Yield: 9.73 g (63%).

### (iv) H-Pro-Pab(OMe)×2 HCl

Boc-Pro-Pab(OMe) (9.7 g, 0.026 mol; see step (iii) above) was dissolved in 250 mL of EtOAc. The ice cooled solution was saturated with HCl(g) by bubbling for 5 min. The product precipitated immediately and 125 mL of absolute ethanol was added. The mixture was sonicated until most of the material had solidified. Diethyl ether (200 mL) was added and the suspension was filtered. A few lumps that had not solidified were again treated with absolute ethanol and diethyl ether. The solid was dried. Yield: 7.57 g (86%).

¹H NMR (400 MHz, CD₃OD) δ 7.74 (d, 2H), 7.58 (d, 2H), 4.55 (s, 2H), 4.38 (m, 1H), 3.98 (s, 3H), 3.45-3.3 (m, 2H), 2.50 (m, 1H), 2.15-2.0 (m, 3H)

### (v) Ph(3-Cl)(5-OCHF₂)—(R)CH(OH)—C(O)-Pro-Pab(OMe)

Ph(3-Cl)(5-OCHF₂)—(R)CH(OH)C(O)OH (50 mg, 0.198 mmol; see Example 1(viii) above), H-Pro-Pab(OMe) (76 mg, 0.218 mmol, see step (iv) above) and PyBOP (115 mg, 0.218 mmol) were dissolved in 2 mL of DMF. Diisopropylethyl amine (104 mg, 0.80 mmol) was added and the mixture was stirred for 2.5 h. The mixture was poured into 50 mL of water and extracted three times with EtOAc and the combined organic phase was washed with brine, dried (Na₂SO₄) and evaporated. The residue was flash chromatographed on silica gel with EtOAc:MeOH (95:5). Yield: 37 mg (36%). Purity: 98%.

¹H NMR (400 MHz, CD₃OD. mixture of rotamers) δ 7.60 (d, 2H, major rotamer), 7.57 (d, 2H, minor rotamer), 7.4-7.1 (m, 5H), 6.89 (t, 1H, major rotamer), 6.87 (t, 1H, minor rotamer), 5.35 (s, 1H, major rotamer), 5.21 (s, 1H, minor rotamer), 4.72 (m, 1H, minor rotamer), 4.5-4.35 (m, 1H and 2H, major rotamer), 4.3-4.25 (m, 2H, minor rotamer), 3.814 (s, 3H, major rotamer), 3.807 (s, 3H, minor rotamer), 3.75-3.5 (m, 1H), 3.35 (m, 1H), 2.2-1.8 (m, 4H)

¹³C-NMR (100 MHz; CD₃OD): (carbonyl and/or amidine carbons, mixture of rotamers) δ 173.3, 173.2, 171.3, 171.0, 153.9, 152.4

APCI-MS: (M+1)=5111/513 m/z

### EXAMPLE 34

### Ph(3-Cl)(5-OCHF₂)—(R)CH(OH)C(O)-Aze-NH—CH₂-((2-amidino)-5-pyridinyl)

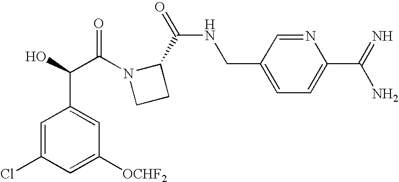

### (i) 6-Cyanonicotinic acid

To a solution of the nicotinic acid N-oxide (51 g, 0.37 mol) in 1.2 L of DMF, NaCN (54 g, 1.1 mol) was added, followed by triethylamine (255 mL, 1.83 mol) and TMSCl (185 mL). The reaction mixture was stirred at 110° C. for 10 h, filtered and the filtrate was concentrated. The residue was dissolved in 100 mL of 2N HCl and extracted with methylene chloride. The organic layers were combined, concentrated and recrystallised from water to yield 12 g (22%) of the product.

### (ii) 5-(Hydroxymethyl)pyridine-2-carbonitrile

To a solution of 6-cyanonicotinic acid (12g, 0.081 mol; see step (i) above) in THF at 0° C., Et₃N (12.4 mL, 0.0892 mol) was added followed by ethyl chloroformate (8.53 mL, 0.0892 mol). The reaction mixture was stirred for 15 min and NaBH₄ (6.14 g, 0.162 mol) was added. Then the mixture was stirred at RT overnight, quenched with water and extracted with methylene chloride. The organic layer was concentrated and purified by column chromatography to yield 4 g (20%) of the alcohol.

### (iii) 5-(Azidomethyl)pyridine-2-carbonitrile

5-(Hydroxymethyl)pyridine-2-carbonitrile (4 g, 0.03 mol; see step (ii) above) was dissolved in 25 mL of methylene chloride and cooled in an ice bath. Mesyl chloride (2.32 mL, 0.0300 mol) and then triethylamine (4.6 mL, 0.033 mol) were added dropwise. The reaction mixture was stirred and after work up the crude mesylate was treated with NaN₃ (7.35 g, 0.113 mol) in 20 mL of DMF. The reaction mixture was stirred at 40° C. for 2 h, diluted with water and extracted with ethyl acetate. The organic layer was concentrated to yield 3.95 g (83%) of the crude azide.

### (iv) 5-(tert-Butoxycarbonylaminomethyl)pyridine-2-carbonitrile

To a solution of 5-(azidomethyl)pyridine-2-carbonitrile (3.95 g, 0.0248 mol; see step (iii) above) in 30 mL of THF and 10 mL of water, triphenyl phosphine (7.8 g, 0.0298 mol) was added and the resultant stirred for 24 h. Then, triethylamine (3.8 mL, 0.027 mol) was added, followed by Boc anhydride (5.4 g, 0.025 mol) and stirring for 2 h. The reaction mixture was partitioned between water and ethyl acetate. The organic layer was concentrated and purified by column chromatography to yield 2.1 g (36%) of the sub-title compound.

¹H NMR (300 MHz, CDCl₃) δ 8.6 (s, 1H), 8.0 (d, 1H), 8.9 (d, 1H), 4.1 (m, 2H), 1.4 (s, 9H)

### (v) 5-(Aminomethyl)pyridine-2-carbonitrile×2 HCl

5-(tert-Butoxycarbonylaminomethyl)pyridine-2-carbonitrile (0.200 g, 0.86 mmol, see step (iv) above) was dissolved in 10 mL of EtOAc saturated with HCl(g) and was stirred for 30 min. The solvent was evaporated and 0.175 g (99%) of the sub-title compound was obtained as its dihydrochloride salt.

¹H NMR (500 MHz, D₂O) δ 8.79 (s, 1H), 8.17 (d, 1H), 8.05 (d, 1H), 4.38 (s, 2H)

### (vi) Boc-Aze-NH—CH₂-5-Py(2-CN)

To a mixture of 5-(aminomethyl)pyridine-2-carbonitrile×2 HCl (0.175 g, 0.85 mmol; see step (v) above), Boc-Aze-OH (0.201 g, 1.00 mmol) and TBTU (0.321 g, 1.00 mmol) in 5 mL of DMF was added dimethylaminopyridine (0.367 g, 3.00 mmol). The mixture was stirred overnight and subsequently poured into water and extracted three times with EtOAc. The combined organic phase was washed with aqueous sodium bicarbonate, dried (Na₂SO₄) and evaporated. The crude product started to crystallise and was used as such in the next step. Yield: 0.23 g (73%).

¹H NMR (500 MHz, CDCl₃) δ 8.66 (s, 1H), 8.2-7.8 (broad, 1H), 7.79 (d, 1H), 7.67 (d, 1H), 4.73 (m, 1H), 4.65-4.5 (m, 2H), 3.94 (m, 1H), 3.81 (m, 1H), 2.6.2.35 (m, 2H), 1.8 (broad, 1H), 1.45 (s, 9H)

### (vii) H-Aze-NH—CH₂-5-Py(2-CN)×2 HCl

Boc-Aze-NH—CH₂-5-Py(2-CN) (0.23 g, 0.73 mmol; see step (vi) above) was dissolved in 10 mL of EtOAc saturated with HCl(g) and was stirred for 30 min. The solvent was evaporated and 0.21 g (100%) of the sub-title compound was obtained as its dihydrochloride salt.

¹H NMR (500 MHz, D₂O) δ 8.64 (s, 1H), 8.0-7.9 (m, 2H), 5.19 (m, 1H), 4.65-4.55 (m, 2H), 4.20 (m, 1H), 4.03 (m, 1H), 2.88 (m, 1H), 2.64 (m, 1H)

### (viii) Ph(3-Cl)(5-OCHF₂)—(R)CH(OH)—C(O)-Aze-NH—CH₂-5-Py(2-CN)

To a mixture of H-Aze-NH—CH₂-5-Py(2-CN)×2 HCl (0.206 g, 0.713 mmol; see step (vii) above), Ph(3-Cl)(5-OCHF₂)—(R)CH(OH)C(O)OH (0.180 g, 0.713 mmol; see Example 1(viii) above) and PyBOP (0.408 g, 0.784 mmol) in 5 mL of DMF was added dimethylaminopyridine (0.367 g, 3.00 mmol). The mixture was stirred overnight and subsequently poured into water and extracted three times with EtOAc. The combined organic phase was washed with aqueous sodium bicarbonate, dried (Na₂SO₄) and evaporated. The crude product was flash chromatographed on silica gel with EtOAc gave a pure product. Yield: 0.197 g (61%).

¹H NMR (500 MHz, CDCl₃) δ 8.63 (m, 1H), 8.22 (bt, 1H), 7.78 (m, 1H), 7.67 (m, 1H), 7.21 (m, 1H), 7.16 (m, 1H), 7.04 (m, 1H), 6.56 (t, 1H), 4.97 (bd, 1H), 4.92 (m, 1H), 4.6-4.5 (m, 2H), 4.40 (bd, 1H), 4.18 (m, 1H), 3.80 (m, 1H), 2.69 (m, 1H), 2.46 (m, 1H), 1.92 (s, 1H)

APCI-MS: (M+1)=451/453 m/z

### (ix) Ph(3-Cl)(5-OCHF₁)—(R)CH(OH)—C(O)-Aze-NH—CH₂-((2-amidino)-5-pyridinyl)×HOAc

Ph(3-Cl)(5-OCHF₂)—(R)CH(OH)—C(O)-Aze-NH—CH₂-5-Py(2-CN) (0.200 g, 0.444 mmol; see step (viii) above), ammonium acetate (1.00 g, 0.0130 mol) and N-acetylcysteine (2.00 g, 0.0122 mol) in 10 mL of methanol was heated at 50° C. for 2 days. Preparative RPLC with CH₃CN:0.1M NH₄OAc (30:79) and running the appropriate fractions again with CH₃CN:0.1M NH₄OAc (5:95-40:60) gave 60 mg (26%) of pure title compound as its acetate salt after freeze drying from water and acetonitrile. Purity: 100%.

¹H NMR (500 MHz, D₂O, mixture of rotamers) δ 8.68 (s, 1H, major rotamer), 8.62 (s, 1H, minor rotamer), 8.05-7.9 (m, 2H), 7.33 (m, 1H, rotamer), 7.27 (m, 1H, rotamer), 7.22 (m, 1H, rotamer), 7.17 (m, 1H, rotamer), 7.01 (m, 1H, rotamer), 6.84 (t, 1H), 5.32 (s, 1H, major rotamer), 5.20 (m, 1H, minor rotamer), 5.13 (s, 1H, minor rotamer), 4.88 (m, 1H, major rotamer), 4.65-4.55 (m, 2H, major rotamer), 4.45-4.35 (m, 1H, rotamer plus 1H, minor rotamer), 4.31 (d, 1H, minor rotamer), 4.2-4.05 (m, 1H plus 1H, rotamer), 2.80 (m, 1H, minor rotamer), 2.61 (m, 1H, major rotamer), 2.33 (m, 1H, major rotamer), 2.24 (m, 1H, minor rotamer), 1.93 (s, 3H)

³C-NMR (100 MHz; D₂O): (carbonyl and/or amidine carbons, mixture of rotamers) δ 181.6, 173.3, 172.7, 172.6, 172.3, 162.6, 162.3

APCI-MS: (M+1)=468/470 m/z

### EXAMPLE 35

### Ph(3-Cl)(5-OCHF₂)—(R)CH(OH)C(O)-Aze-NH—CH₂-((2-methoxyamidino)-5-pyridinyl)

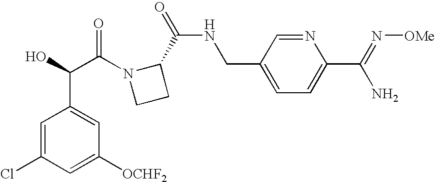

### (i) Boc-NH—CH₂-[(2-(amino(hydroxylimino)methyl))-5-pyridinyl]

5-(tert-Butoxycarbonylaminomethyl)pyridine-2-carbonitrile (1.00 g, 4.29 mmol; see Example 34(iv) above) was dissolved in 10 mL of ethanol and hydroxylamine hydrochloride (0.894 g, 0.0129 mol) and triethyl amine (1.30 g, 0.0129 mol) were added.

The mixture was stirred at room temperature for 6 days. The mixture was partitioned between water and methylene chloride. The aqueous layer was extracted with methylene chloride and the combined organic phase was washed with water, dried (Na₂SO₄) and evaporated. Yield: 0.96 g (84%).

¹H NMR (400 MHz, acetone-d₆) δ 9.01 (bs, 1H), 8.50 (bs, 1H), 7.87 (m, 1H), 7.70 (m, 1H), 6.58 (broad, 1H), 5.70 (broad, 2H), 4.31 (d, 2H), 1.41 (s, 9H)

### (ii) Boc-Aze-NH—CH₂-(2-(amidino)-5-pyridinyl)×HOAC

This reaction was carried out according to the method described in Judkins et al, Synth. Comm. (1998) 4351. A suspension of Boc-NH—CH₂-[(2-(amino(hydroxylimino)methyl))-5-pyridinyl] (0.910 g, 3.42 mmol; see step (i) above), acetic anhydride (0.35 mL, 3.7 mmol) and 0.35 g of 10% Pd/C (50% moisture) in 100 mL of acetic acid was hydrogenated at a pressure of 5 atm. for 5 h. The mixture was filtered through Celite and concentrated. The residue was freeze-dried from water and acetonitrile to give 0.97 g (92%) of the sub-title compound.

¹H NMR (500 MHz, CD₃OD) δ 8.74 (s, 1H), 8.12 (d, 1H), 7.98 (d, 1H), 4.38 (s, 2H), 1.92 (s, 3H), 1.46 (s, 9H)

### (iii) Boc-NH—CH₂-(2-(amino(trimethylsilylethylimino)methyl)-5-pyridinyl)

To a suspension of Boc-NH—CH₂-(2-(amidino)-5-pyridinyl)×HOAc (0.96 g, 3.1 mmol; see step (ii) above) in 75 mL of THF was added a solution of potassium carbonate (1.07 g, 7.7 mmol) and Teoc-p-nitrophenyl carbonate (1.14 g, 4.02 mmol) in 15 mL of water. The mixture was stirred overnight. An excess of glycine and potassium carbonate was added, and the reaction was continued for 2 h. The THF was evaporated and the remainder was extracted three times with EtOAc. The combined organic phase was washed with water, dried (Na₂SO₄) and evaporated. The product could be used without further purification.

¹H NMR (500 MHz, CDCl₃) δ 9.31 (broad, 1H), 8.52 (s, 1H), 8.41 (d, 1H), 8.35 (broad, 1H), 7.74 (d, 1H), 4.97 (broad, 1H), 4.39 (m, 2H), 4.26 (m, 2H), 1.46 (s, 9H), 1.14 (m, 2H), 0.07 (s, 9H)

### (iv) H₂N—CH₂-(2-(amino(trimethylsilylethylimino)methyl)-5-pyridinyl)×2 HCl

Boc-NH—CH₂-(2-(amino(trimethylsilylethylimino)methyl)-5-pyridinyl) (0.23 g, 0.58 mmol; see step (iii) above) was dissolved in 25 mL of EtOAc saturated with HCl(g) and stirred for 30 min. The solvent was evaporated and the product used without further purification. Yield: 0.21 g (98%).

¹H NMR (500 MHz, D₂O) δ 8.89 (s, 1H), 8.25 (s, 2H), 4.55 (m, 2H), 4.42 (s, 2H), 1.20 (m, 2H), 0.09 (s, 9H)

### (v) Boc-Aze-NH—CH₂-(2-(amino(trimethylsilylethylimino)methyl)-5-pyridinyl)

To a solution of H₂N—CH₂-(2-(amino(trimethylsilylethylimino)methyl)-5-pyridinyl)×2 HCl (0.21 g, 0.57 mmol; see step (iv) above), Boc-Aze-OH (0.127 g, 0.631 mmol), and TBTU (233 mg, 0.726 mmol) in 5 mL of DMF was added dimethylaminopyridine (269 mg, 2.20 mmol). The mixture was stirred overnight, poured into 100 mL of water and extracted with EtOAc three times. The combined organic phase was washed with aqueous sodium bicarbonate and water, dried (Na₂SO₄) and evaporated. The crude product was flash chromatographed on silica gel with EtOAc to give 170 mg (56%) of the desired product.

¹H NMR (500 MHz, CDCl₃) δ 9.33 (broad, 1H), 8.54 (s, 1H), 8.41 (d, 1H), 8.36 (broad, 1H), 7.75 (m, 1H), 4.72 (m, 1H), 4.56 (m, 2H), 4.26 (m, 2H), 3.93 (m, 1H), 3.80 (m, 1H), 2.6-2.4 (m, 2H), 1.42 (s, 9H), 1.14 (m, 2H), 0.07 (s, 9H)

### (vi) H-Aze-NH—CH₂(2-(amino(trimethylsilylethylimino)methyl)-5-pyridinyl)×2 HCl

Boc-Aze-NH—CH₂-(2-(amino(trimethylsilylethylimino)methyl)-5-pyridinyl) (170 mg, 0.356 mmol; see step (v) above) was dissolved in 25 mL of EtOAc saturated with HCl(g) and stirred for 30 min. The solvent was evaporated and the product used without further purification. Yield: 160 mg (100%).

¹H NMR (500 MHz, CD₃OD) δ 9.00 (m, 1H), 8.84 (m, 1H), 8.23 (d, 2H), 8.10 (m, 1H), 5.09 (m, 1H), 4.7-4.6 (m, 2H), 4.51 (m, 2H), 4.14 (m, 1H), 3.97 (m, 1H), 2.86 (m, 1H), 2.58 (m, 1H), 1.22 (m, 2H), 0.11 (s, 9H)

### (vii) Ph(3-Cl)(5-OCHF₂)—(R)CH(OH)C(O)-Aze-NH—CH₂-(2-(amino(tri-methylsilylethylimino)methyl)-5-pyridinyl)

To a solution of H-Aze-NH—CH₂-(2-(amino(trimethylsilylethylimino)-methyl)-5-pyridinyl)×2 HCl (160 mg, 0.462 mmol; see step (vi) above), Ph(3-Cl)(5-OCHF₂)—(R)CH(OH)C(O)OH (131 mg, 0.462 mmol; see Example 1(viii) above) and PyBOP (263 mg, 0.505 mmol) in 5 mL of DMF was added diisopropylethyl amine (0.30 mL, 1.71 mmol). The mixture was stirred overnight, poured into 100 mL of water and extracted three times with EtOAc. The combined organic phase was washed with aqueous sodium bicarbonate and water, dried (Na₂SO₄) and evaporated. The crude product was flash chromatographed on silica gel with EtOAc:MeOH (95:5) to give 148 mg (52%) of the desired product.

### (viii) Ph(3-Cl)(5-OCHF₂)—(R)CH(OH)C(O)-Aze-NH—CH₂-(2-(methoxy-amino(trimethylsilylethylimino)methyl)-5-pyridinyl)

A suspension of Ph(3-Cl)(5-OCHF₂)—(R)CH(OH)—C(O)-Aze-NH—CH₂-(2-(methoxyamino(trimethylsilylethylimino)methyl)-5-pyridinyl) (148 mg, 0.242 mmol; see step (vii) above) and 0-methylhydroxyl amine (202 mg, 2.42 mmol) in 10 mL of acetonitrile was heated at 70° C. for 3 h. The mixture was partitioned between water and EtOAc. The aqueous layer was extracted twice with EtOAc and the combined organic phase was washed with water, dried (Na₂SO₄) and evaporated. The crude material was flash chromatographed on silica gel with EtOAc:MeOH (95:5) to give 44 mg (28%) of pure material.

¹H NMR (500 MHz, CDCl₃) δ 8.55 (m, 1H), 8.05 (bt, 1H), 7.70 (m, 1H), 7.58 (s, 1H), 7.56 (d, 1H), 7.22 (m, 1H), 7.16 (m, 1H), 7.03 (m, 1H), 6.50 (t, 1H), 4.92 (s, 1H), 4.89 (m, 1H), 4.55-4.45 (m, 2H), 4.38 (broad, 1H), 4.2-4.1 (m, 3H), 4.00 (s, 3H), 3.73 (m, 1H), 2.69 (m, 1H), 2.44 (m, 1H), 0.97 (m, 2H), 0.02 (s, 9H)

### (ix) Ph(3-Cl)(5-OCHF₂)—(R)CH(OH)C(O)-Aze-NH—CH₂-((2-methoxy-amidino)-5-pyridinyl)

Ph(3-Cl)(5-OCHF₂)—(R)CH(OH)C(O)-Aze-NH—CH₂-(2-(methoxyamino(tri-methylsilylethylimino)methyl)-5-pyridinyl) (44 mg, 0.069 mmol; see step (viii) above) was dissolved in 2 mL of TFA and allowed to react for 1 h. The TFA was evaporated and the residue was partitioned between EtOAc and aqueous sodium bicarbonate. The aqueous layer was extracted with EtOAc and the combined organic phase was washed with water, dried (Na₂SO₄) and evaporated. Yield: 30 mg (88%). Purity: >95%.

¹H NMR (500 MHz, CDCl₃) δ 8.44 (m, 1H), 8.03 (bt, 1H), 7.91 (m, 1H), 7.60 (m, 1H), 7.19 (m, 1H), 7.13 (m, 1H), 7.00 (m, 1H), 6.52 (t, 1H), 5.6-5.45 (broad, 2H), 4.90 (s, 1H), 4.89 (m, 1H), 4.55-4.4 (m, 2H), 4.27 (broad, 1H), 4.12 (m, 1H), 3.92 (s, 3 H), 2.68 (m, 1H), 2.41 (m, 1H)

¹³C-NMR (100 MHz; CDCl₃): (carbonyl and/or amidine carbons) δ 173.0, 170.9, 152.6

APCI-MS: (M+1)=498/500 m/z

### EXAMPLE 36

### Ph(3-Cl)(5-OCHF₂)—(R)CH(OH)C(O)-Aze-NH—CH₂-((5-amidino)-2-pyrimidinyl)

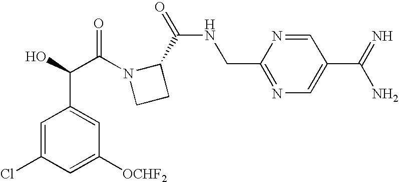

### (i) 2-Amino-2-iminoethylcarbamate.AcOH

N-Boc-aminoacetonitrile (40.2 g, 257.4 mmol) and N-acetylcysteine (42.0 g, 257.4 mmol) were dissolved in methanol (300 mL) at 60° C. and ammonia was passed through for 18 h. The solvent was removed in vacuo. After ion exchange chromatography (Amberlite IRA-400 (AcOH)) and recrystallisation from acetone, 28.4 g (53%) of the sub-title compound was obtained as a white solid.

¹H NMR (300 MHz, CD₃OD) δ 4.41 (t, J=4.9 Hz, 1H), 4.01 (s, 2H), 2.91 (d, J=5.0 Hz, 2H), 2.01 (s, 3H), 1.46 (s, 9H)

### (ii) 1,3-Bis(dimethylamino)-2-cyanotrimethinium perchlorate

A solution of 3-dimethylaminoacrylonitrile (25.0 g, 260.0 mmol) in chloroform (75 mL) was added dropwise to a solution of (chloromethylene)dimethylammonium chloride (50.0 g, 390.1 mmol) in chloroform (175 mL) at 0° C. The reaction mixture was stirred an additional 2 h at 0° C., then allowed to warm to room temperature overnight, then subsequently heated for 8 h under reflux. The solvent was removed in vacuo. The residue was added to a mixture of sodium perchlorate (110 g, 0.898 mmol) in water (150 mL) and ethanol (300 mL). The mixture was heated under reflux for 15 min then cooled and allowed to stand overnight in a refrigerator. The precipitate was collected and recrystallized from ethanol to yield 23.8 g (52%) of the sub-title compound as colorless needles.

mp: 140-141° C.

¹H NMR (300 MHz, CDCl₃) δ 8.24 (s, 2H), 3.59 (s, 6H), 3.51 (s, 6H)

### (iii) Boc-NH—CH₂-(5-cyano)-2-pyrimidine

A mixture of t-butyl 2-amino-2-iminoethylcarbamate-AcOH (5.0 g, 23.8 mmol; see step (i) above) and 1,3-bis(dimethylamino)-2-cyanotrimethinium perchlorate (6.0 g, 23.8 mmol; see step (ii) above) in pyridine (300 mL) was stirred under nitrogen at 70-75° C. for 16 h and then heated under reflux for 6 h. The mixture was cooled to room temperature and the solvent was removed in vacuo. The residue was extracted with a hot mixture (1:1) of ethyl acetate and chloroform, filtered through a small pad of silica, and concentrated to give the crude product. Flash chromatography on silica eluting with chloroform gave 4.0 g (71%) of the title compound as colorless oil, which solidified upon standing.

mp: 86-87° C.

R_{f}=0.77 (silica, 3:2 Ethyl Acetate/Chloroform)

¹H NMR (300 MHz, DMSO-d₆) δ 9.25 (s, 2H), 7.39 (bt, 1H), 4.39 (d, J=6 Hz, 2H), 1.38 (s, 9H).

¹³C NMR (750 MHz, DMSO-d₆) δ 170.4, 160.3, 155.8, 115.2, 106.9, 80.0, 46.3, 28.1

APCI-MS: (M+1)=235 m/z

### (iv) Boc-Aze-NH—CH₂-((5-cyano)-2-pyrimidinyl)

Boc-NH—CH₂-(5-cyano)-2-pyrimidine (1.14 g, 4.87 mmol; see step (iii) above) was dissolved in 50 mL of EtOAc saturated with HCl(g) and allowed to react for 1 h and concentrated. The residue was dissolved in 20 mL of DMF and cooled in an ice bath. Diisopropylethyl amine (3.5 mL, 0.020 mol), Boc-Aze-OH (1.08 g, 5.37 mmol) and HATU (2.80 g, 5.38 mmol) were added and the reaction mixture was stirred at room temperature overnight. The solvent was evaporated and the product was purified by preparative RPLC using CH₃CN:0.1M NH₄OAc (40:60). The acetonitrile was evaporated and the aqueous layer was extracted three times with EtOAc. The combined organic layer was dried (MgSO₄) and evaporated. Yield: 1.12 g (72%).

¹H NMR (400 MHz, CDCl₃) δ 8.95 (s, 2H), 4.82 (d, 2H), 4.74 (m, 1H), 3.95 (m, 1H), 3.84 (m, 1H), 2.6-2.4 (m, 2H), 1.47 (s, 9H)

### (v) Boc-Aze-NH—CH₂-((5-amidino)-2-pyrimidinyl)×HOAc

A solution of Boc-Aze-NH—CH₂-((5-cyano)-2-pyrimidinyl) (0.83 g, 2.6 mmol; see step (iv) above), N-acetylcysteine (0.43 g, 2.6 mmol) and ammonium acetate (0.60 g, 7.8 mmol) in 10 mL of methanol was heated at 60° C. under nitrogen for 2 days. The solvent was evaporated and the crude material was purified by preparative RPLC using a gradient of CH₃CN:0.1M NH₄OAc (5:95 to 100:0). The fractions of interest were freeze dried to give 1.0 g (93%) of the desired material.

¹H NMR (300 MHz, D₂O, signals obscured by the HDO signal) δ 9.17 (s, 2H), 4.1-3.9 (m, 2H), 2.60 (m, 1H), 2.29 (m, 1H), 1.93 (s, 3H), 1.44 (s, 9H)

### (vi) Boc-Aze-NH—CH₂-[(5-(amino(trimethylsilylethylimino)methyl))-2-pyrimidinyl]

To a suspension of Boc-Aze-NH—CH₂-((5-amidino)-2-pyrimidinyl)×HOAc (0.95 g, 2.41 mmol; see step (v) above) in 50 mL of THF was added a solution of Teoc-p-nitrophenyl carbonate (0.85 g, 3.0 mmol) and potassium carbonate (1.0 g, 7.2 mmol) in 10 mL of water. The mixture was stirred for 24 h, concentrated and partitioned between water and methylene chloride. The organic layer was washed twice with saturated aqueous sodium bicarbonate, dried (Na₂SO₄) and evaporated. The crude product was flash chromatographed on silica gel with heptane:EtOAc (1:1). Yield: 1.04 g (90%).

¹H NMR (300 MHz, CDCl₃) δ 9.16 (s, 2H), 4.80 (d, 2H), 4.73 (m, 1H), 4.26 (m, 2H), 4.0-3.8 (m, 2H), 2.6-2.4 (m, 2H), 1.47 (s, 9H), 1.12 (m, 2H), 0.07 (s, 9H)

### (vii) Ph(3-Cl)(5-OCHF₂)—(R)CH(OH)C(O)-Aze-NH—CH₂-[(5-(amino(tri-methylsilylethylimino)methyl))-2-pyrimidinyl]

Boc-Aze-NH—CH₂-[(5-(amino(trimethylsilylethylimino)methyl))-2-pyrimidinyl] (0.209 g, 0.437 mmol; see step (vi) above) was dissolved in 25 mL of EtOAc saturated with HCl(g) and allowed to react for 15 min. The solvent was evaporated and the remainder was dissolved in 4 mL of DMF. Ph(3-Cl)(5-OCHF₂)—(R)CH(OH)C(O)OH (0.100 g, 0.396 mmol; see Example 1(viii) above), PyBOP (0.231 g, 0.444 mmol) and diisopropylethyl amine (0.208 g, 1.61 mmol) were added, and the mixture was stirred for 80 min. The reaction mixture was poured into 100 mL of water and extracted three times with EtOAc. The combined organic layer was washed with brine, dried (Na₂SO₄) and evaporated. The crude product was purified by preparative RPLC using CH₃CN:0.1M NH₄OAc (1:1).

Yield: 63 mg (26%).

¹H NMR (400 MHz, CDCl₃, mixture of rotamers) δ 9.3 (broad, 1H), 9.03 (s, 2H, minor rotamer), 9.00 (s, 2H, major rotamer), 8.25 (m, 1H, major rotamer), 7.9 (broad, 1H), 7.80 (m, 1H, minor rotamer), 7.2-6.9 (m, 3H), 6.50 (t, 1H), 5.14 (s, 1H, minor rotamer), 5.08 (m, 1H, minor rotamer), 4.94 (s, 1H, major rotamer), 4.80 (m, 1H, major rotamer), 4.7-4.4 (m, 2H), 4.3-3.9 (m, 3H), 3.74 (m, 1H, major rotamer), 2.7-2.1 (m, 2H), 1.03 (m, 2H), 0.01 (s, 9H)

### (viii) Ph(3-Cl)(5-OCHF₂)—(R)CH(OH)C(O)-Aze-NH—CH₂-((5-amidino)-2-pyrimidinyl)×TFA

Ph(3-Cl)(5-OCHF₂)—(R)CH(OH)C(O)-Aze-NH—CH₂-[(5-(amino(trimethyl-silylethylimino)methyl))-2-pyrimidinyl] (21 mg, 0.034 mmol; see step (vii) above) was dissolved in 0.5 mL of methylene chloride and cooled in an ice bath. TFA (2 mL) was added and the mixture was stirred for 60 min and then concentrated. The product was freeze-dried from water and acetonitrile. Yield: 20 mg (100%). Purity: 100%.

¹H NMR (400 MHz, CD₃OD, mixture of rotamer, signals obscured by the HDO signal) δ 9.08 (s, 2H), 7.4-7.1 (m, 3H), 6.88 (t, 1H, major rotamer), 6.85 (t, 1H, minor rotamer), 5.30 (m, 1H, minor rotamer), 5.22 (s, 1H, minor rotamer), 5.20 (s, 1H, major rotamer), 4.73 (m, 1H, major rotamer), 4.34 (m, 1H, rotamer), 4.21 (m, 1H, rotamer), 4.15-3.95 (m, 2H, rotamers), 2.73 (m, 1H, rotamer), 2.57 (m, 1H, rotamer), 2.45-2.25 (m, 2H, rotamers)

¹³C-NMR (100 MHz; CD₃OD): (carbonyl and/or amidine carbons, mixture of rotamers) δ 173.0, 172.6, 172.1, 171.0, 163.4.

APCI-MS: (M+1)=469/471 m/z

### EXAMPLE 37

### Ph(3-Cl)(5-OCHF₂)—(R)CH(OH)C(O)-Aze-NH—CH₂-((5-methoxyamidino)-2-pyrimidinyl)

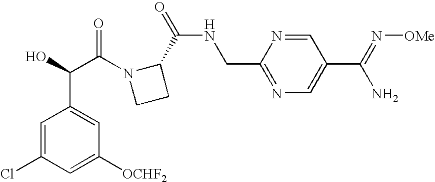

### (i) Ph(3-Cl)(5-OCHF₂)—(R)CH(OH)C(O)-Aze-NH—CH₂-[(5-(methoxyamino-(trimethylsilylethylimino)methyl))-2-pyrimidinyl]

A suspension of Ph(3-Cl)(5-OCHF₂)—(R)CH(OH)C(O)-Aze-NH—CH₂-[(5-(amino(trimethylsilylethylimino)methyl))-2-pyrimidinyl] (40 mg, 0.065 mmol; see Example 36(vii) above) and O-methylhydroxyl amine (33 mg, 0.40 mmol) in 3 mL of acetonitrile was heated at 70° C. for 3 h. The mixture was partitioned between water and EtOAc. The aqueous layer was extracted twice with EtOAc and the combined organic phase was washed with water, dried (Na₂SO₄) and evaporated. Yield: 33 mg (79%).

¹H NMR (400 MHz, CDCl₃, mixture of rotamers) δ 8.76 (s 2H, major rotamer), 8.70 (s, 2H, rotamer), 8.18 (m, 1H), 7.62 (s, 1H), 7.4-6.9 (m, 4H), 6.50 (bt, 1H), 5.3-4.5 (m, 4H), 4.2-4.05 (m, 3H), 3.96 (s, 3H), 3.68 (m, 1H), 2.8-2.2 (m, 2H), 2.1 (broad, 1H), 0.96 (m, 2H), 0.01 (s, 9H)

### (ii) Ph(3-Cl)(5-OCHF₂)—(R)CH(OH)C(O)-Aze-NH—CH₂-((5-methoxy-amidino)-2-pyrimidinyl)

Ph(3-Cl)(5-OCHF₂)—(R)CH(OH)C(O)-Aze-NH—CH₂-[(5-(methoxyamino-(trimethylsilylethylimino)methyl))-2-pyrimidinyl] (33 mg, 0.052 mmol; see step (i) above) was dissolved in 0.5 mL of methylene chloride and cooled in an ice bath. TFA (2 mL) was added and the mixture was stirred for 2 h and then concentrated. The product was freeze dried from water and acetonitrile. Yield: 31 mg (81%). Purity: 100%.

¹H NMR (400 MHz, CD₃OD, mixture of rotamer, signals obscured by the HDO signal) 8.96 (s, 2H, rotamer), 8.94 (s, 2H, rotamer), 7.4-7.3 (m, 1H), 7.2-7.1 (m, 2H), 6.88 (t, 1H, rotamer), 6.85 (t, 1H, rotamer), 5.29 (m, 1H, rotamer), 5.24 (s, 1H, rotamer), 5.20 (s, 1H, rotamer), 4.75-4.55 (m, 2H), 4.33 (m, 1H, rotamer), 4.19 (m, 1H, rotamer), 4.15-3.95 (m, 2H, rotamers), 3.88 (s, 3H, rotamer), 3.86 (s, 3H, rotamer), 2.72 (m, 1H, rotamer), 2.56 (m, 1H, rotamer), 2.45-2.25 (m, 2H, rotamers)

¹³C-NMR (100 MHz; CD₃OD): (carbonyl and/or amidine carbons, mixture of rotamers) δ 172.8, 172.6, 172.1, 171.8, 167.8, 167.7, 155.1, 152.3, 152.1

APCI-MS: (M+1)=499/501 m/z

### EXAMPLE 38

### Ph(3-Cl)(5-OCHF₂)—(R)CH(OH)C(O)-Aze-Pab(3-F)

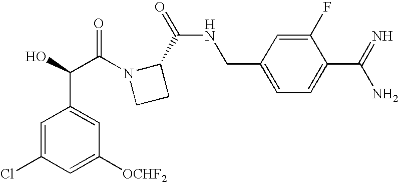

### (i) 2-Fluoro-4-vinylbenzonitrile

A solution of 4-bromo-2-fluorobenzonitrile (4.92 g, 0.0246 mol), vinyl tributyltin (0.78 g, 0.246 mol), and tetrakistriphenylphosphine (0.67 g, 0.58 mmol) in 250 mL of toluene was refluxed under nitrogen overnight. The solvent was evaporated and the residue was flash chromatographed on silica gel with heptane:CH₂Cl₂ (1:1) to pure CH₂Cl₂. A colourless oil was obtained that crystallised. Yield: 3.0 g (82%).

¹H NMR (300 MHz, CDCl₃) δ 7.56 (m, 1H), 7.3-7.2 (m, 2H), 6.69 (m, 1H), 5.89 (d, 1H), 5.51 (d, 1H)

### (ii) 2-Fluoro-4-hydroxymethylbenzonitrile

Into a cooled solution (−78° C.) of 2-fluoro-4-vinylbenzonitrile (1.3 g, 8.8 mmol; see step (i) above) in 40 mL of CH₂Cl₂ and 5 mL of methanol was bubbled ozone (50 L/h, 29 g/m³) for 30 min. Argon was subsequently bubbled through to remove excess ozone. Sodium borohydride (0.67 g, 0.018 mol) was added and the cooling bath was removed. The mixture was stirred and allowed to react for 1 h. The mixture was evaporated and 2M HCl was added. The mixture was extracted twice with diethyl ether and the combined ether fraction was dried (Na₂SO₄) and evaporated. The crude product crystallised. Yield: 1.1 g (81%).

¹H NMR (300 MHz, CDCl₃) δ 7.59 (m, 1H), 7.3-7.2 (m, 2H), 4.79 (d, 2H), 2.26 (t, 1H)

### (iii) 4-Cyano-3-fluorobenzyl methanesulfonate

2-Fluoro-4-hydroxymethylbenzonitrile (1.3 g, 8.6 mmol; see step (ii) above) was dissolved in 50 mL of CH₂Cl₂ and cooled on an ice bath. Triethylamine (0.87 g, 8.6 mmol) and methanesulfonyl chloride (0.99 g, 8.7 mmol) were added. After stirring for 1.5 h the reaction mixture was washed with 1M HCl. The organic phase was dried (Na₂SO₄) and evaporated. The product could be used without purification. Yield of a colourless oil: 1.8 g (92%).

¹H NMR (400 MHz, CDCl₃) δ 7.66 (m, 1H), 7.35-7.3 (m, 2H), 5.26 (s, 2H), 3.07 (s, 3H)

### (iv) 4-Azidomethyl-2-fluorobenzonitrile

To an ice cooled solution of 4-cyano-3-fluorobenzyl methanesulfonate (1.8 g, 7.9 mmol; see step (iii) above) was added sodium azide (0.80 g, 0.012 mol). The mixture was stirred overnight and then poured into 200 mL of water and extracted three times with diethyl ether. The combined ethereal phase was washed five times with water, dried (Na₂SO₄) and evaporated. The crude colourless oil could be used without further purification. Yield: 1.2 g (87%).

¹H NMR (300 MHz, CDCl₃) δ 7.64 (m, 1H), 7.25-7.18 (m, 2H), 4.47 (s, 2H)

### (v) 4-Aminomethyl-2-fluorobenzonitrile

To a suspension of stannous chloride dihydrate (0.45 g, 2.4 mmol) in 20 mL of acetonitrile under stirring was added thiophenol (1.07 g, 9.7 mmol) and triethylamine (0.726 g, 7.17 mmol). Thereafter was added a solution of 4-azidomethyl-2-fluorobenzonitrile (0.279 g, 1.58 mmol; see step (iv) above) in a few mLs of acetonitrile. After 1.5 h, the azide was consumed and the solvent was evaporated. The residue was dissolved in methylene chloride and washed three times with 2M NaOH. The organic phase was extracted twice with 1M HCl. The combined acidic aqueous phase was washed with methylene chloride and then made alkaline with 2M NaOH and extracted three times with methylene chloride. The organic phase was dried (Na₂SO₄) and evaporated to give 0.172 g (72%) of the desired sub-title compound which could be used without purification.

¹H NMR (400 MHz, CDCl₃) δ 7.58 (m, 1H), 7.3-7.2 (m, 2H), 3.98 (s, 2H), 1.55-1.35 (broad, 2H)

### (vi) Boc-Aze-NHCH₂-Ph(3-F, 4-CN)

To an ice cooled solution of Boc-Aze-OH (0.194 g, 0.96 mmol) in 5 mL of DMF was added TBTU (0.50 g, 9.6 mmol). After 30 min another solution, comprising 4-aminomethyl-2-fluorobenzonitrile (0.17 g, 0.81 mmol; see step (v) above) and diiisopropylethyl amine (0.326 g, 2.53 mmol) in 7 mL of DMF was added. The resulting solution was stirred overnight at room temperature. The solvent was evaporated and the product was purified by preparative RPLC using CH₃CN:0.1M NH₄OAc (50:50). Freeze-drying gave 0.237 g (74%) of the desired sub-title compound.

¹H NMR (300 MHz, CD₃OD) δ 7.70 (m, 1H), 7.35-7.25 (m, 2H), 4.65-4.35 (m, 3H), 4.0-3.85 (m, 2H), 2.51 (m, 1H), 2.19 (m, 1H), 1.40 (s, 9H)

### (vii) Ph(3-Cl)(5-OCHF₂)—(R)CH(OH)C(O)-Aze-NHCH₂-Ph(3-F, 4-CN)

Boc-Aze-NHCH₂-Ph(3-F, 4-CN) (0.118 g, 0.354 mmol; from step (vi) above) was dissolved in 30 mL of EtOAc saturated with HCl(g). The reaction was stirred for 20 min and evaporated. The resulting dihydrochloride and HATU (0.152 g, 0.400 mmol) were dissolved in 5 mL of DMF. That solution was added to an ice cooled solution of Ph(3-Cl)(5-OCHF₂)—(R)CH(OH)C(O)OH (0.101 g, 0.400 mmol; see Example 1(viii) above) in 5 mL of DMF. The reaction was stirred overnight at ambient temperature. The solvent was evaporated and the product was purified by preparative RPLC with CH₃CN:0.1M NH₄OAc (50:50). Freeze-drying gave 0.130 g (77%) of the desired sub-title compound.

¹H NMR (500 MHz, CD₃OD mixture of rotamers) δ 7.7-7.6 (m, 1H), 7.35-7.1 (m, 5H), 6.88 (t, 1H, rotamer), 6.86 (t, 1H, rotamer), 5.25-5.1 (m, 1H plus minor rotamer from the following proton), 4.80 (m, 1H, major rotamer), 4.6-4.4 (m, 2H), 4.36 (m, 1H, major rotamer), 4.18 (m, 1H, major rotamer), 4.07 (m, 1H, minor rotamer), 3.98 (m, 1H, minor rotamer), 2.70 (m, 1H, minor rotamer), 2.53 (m, 1H, major rotamer), 2.29 (m, 1H, major rotamer), 2.16 (m, 1H, minor rotamer)

### (viii) Ph(3-Cl)(5-OCHF₂)—(R)CH(OH)C(O)-Aze-Pab(3-F)

Ph(3-Cl)(5-OCHF₂)—(R)CH(OH)C(O)-Aze-NHCH₂-Ph(3-F, 4-CN) (0.130 g, 0.278 mmol; see step (vii) above) was dissolved in 80 mL of ethanol saturated with HCl(g). The mixture was allowed to react at room temperature overnight. The solvent was evaporated and the residue was re-dissolved in 100 mL of ethanol saturated with NH₃(g). The reaction was allowed to proceed slowly at room temperature for two days. The temperature was raised to 50° C. and the reaction continued for another 3 days. The starting material was consumed and the solvent was evaporated. The product was purified by preparative RPLC and freeze-dried to give 17 mg (13%) of the title compound as its HOAc salt.

¹H NMR (600 MHz, CD₃OD mixture of rotamers) δ 7.65-7.6 (m, 1H), 7.4-7.3 (m, 3H), 7.25-7.1 (m, 2H), 7.15-6.7 (m, 1H), 5.25-5.1 (m, 1H plus minor rotamer of the following proton), 4.8 (m, 1H, major rotamer partially hidden by CD₃OH), 4.6-3.95 (m, 4H), 2.69 (m, 1H, minor rotamer), 2.56 (m, 1H, major rotamer), 2.28 (m, 1H, major rotamer), 2.14 (m, 1H, minor rotamer), 1.90 (s, 3H)

¹³C-NMR (100 MHz; CD₃OD): (carbonyl and/or amidine carbons, mixture of rotamers) δ 180.6, 173.4, 173.1, 172.9, 164.5, 162.3, 159.8

APCI-MS: (M+1)=485/487 m/z

### EXAMPLE 39

### Ph(3-Cl)(5-OCHF₂)—(R)CH(OH)C(O)-Aze-Pab(2.6-diF)

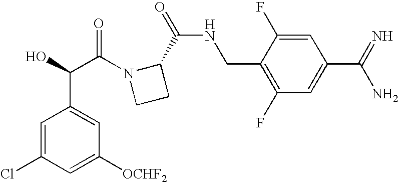

### (i) 2,6-Difluoro-4[(methylsulfinyl)(methylthio)methyl]benzonitrile

(Methylsulfinyl)(methylthio)methane (7.26g, 0.0584 mol) was dissolved in 100 mL of dry THF under argon and was cooled to −78° C. Butyllithium in hexane (16 mL 1.6M, 0.0256 mol) was added dropwise with stirring. The mixture was stirred for 15 min. Meanwhile, a solution of 3,4,5-trifluorobenzonitrile (4.0 g, 0.025 mmol) in 100 mL of dry THF was cooled to −78° C. under argon and the former solution was added through a cannula to the latter solution over a period of 35 min. After 30 min, the cooling bath was removed and when the reaction had reached room temperature it was poured into 400 mL of water. The THF was evaporated and the remaining aqueous layer was extracted three times with diethyl ether. The combined ether phase was washed with water, dried (Na₂SO₄) and evaporated. Yield: 2.0 g (30%).

¹H NMR (500 MHz, CDCl₃) δ 7.4-7.25 (m, 2H), 5.01 (s, 1H, diasteromer), 4.91 (s, 1H, diasteromer), 2.88 (s, 3H, diasteromer), 2.52 (s, 3H, diasteromer), 2.49 (s, 3H, diasteromer), 2.34 (s, 3H, diasteromer), 1.72 (broad, 1H)

### (ii) 2,6-Difluoro-4-formylbenzonitrile

2,6-Difluoro4[(methylsulfinyl)(methylthio)methyl]benzonitrile (2.17 g, 8.32 mmol; see step (i) above) was dissolved in 90 mL of THF and 3.5 mL of concentrated sulfuric acid was added. The mixture was left at room temperature for 3 days and subsequently poured into 450 mL of water. Extraction three times with EtOAc followed and the combined ethereal phase was washed twice with aqueous sodium bicarbonate and with brine, dried (Na₂SO₄) and evaporated. Yield: 1.36 g (98%). The position of the formyl group was established by ¹³C NMR. The signal from the fluorinated carbons at 162.7 ppm exhibited the expected coupling pattern with two coupling constants in the order of 260 Hz and 6.3 Hz respectively corresponding to an ipso and a meta coupling from the fluorine atoms.

¹H NMR (400 MHz, CDCl₃) δ 10.35 (s, 1H), 7.33 (m, 2H)

### (iii) 2,6-Difluoro-4-hydroxymethylbenzonitrile

2,6-Difluoro-4-formylbenzonitrile (1.36 g, 8.13 mmol; see step (ii) above) was dissolved in 25 mL of methanol and cooled on an ice bath. Sodium borohydride (0.307 g, 8.12 mmol) was added in portions with stirring and the reaction was left for 65 min. The solvent was evaporated and the residue was partitioned between diethyl ether and aqueous sodium bicarbonate. The ethereal layer was washed with more aqueous sodium bicarbonate and brine, dried (Na₂SO₄) and evaporated. The crude product crystallised soon and could be used without further purification. Yield: 1.24 g (90%).

¹H NMR (400 MHz, CDCl₃) δ 7.24 (m, 2H), 4.81 (s, 2H), 2.10 (broad, 1H)

### (iv) 4-Cyano-2,6-difluorobenzyl methanesulfonate

To an ice cooled solution of 2,6-difluoro-4-hydroxymethylbenzonitrile (1.24 g, 7.32 mmol; see step (iii) above) and methanesulfonyl chloride (0.93 g, 8.1 mmol) in 60 mL of methylene chloride was added triethylamine (0.81 g, 8.1 mmol) with stirring. After 3 h at 0° C., the mixture was washed twice with 1M HCl and once with water, dried (Na₂SO₄) and evaporated. The product could be used without further purification. Yield: 1.61 g (89%).

¹H NMR (300 MHz, CDCl₃) δ 7.29 (m, 2H), 5.33 (s, 2H), 3.07 (s, 3H)

### (v) 4-Azidomethyl-2,6-difluorobenzonitrile

A mixture of 4-cyano-2,6-difluorobenzyl methanesulfonate (1.61 g, 6.51 mmol; see step (iv) above) and sodium azide (0.72 g, 0.0111 mol) in 10 mL of water and 20 mL of DMF was stirred at room temperature overnight. The resultant was subsequently poured into 200 mL of water and extracted three times with diethyl ether. The combined ethereal phase was washed five times with water, dried (Na₂SO₄) and evaporated. A small sample was evaporated for NMR purposes and the product crystallised. The rest was evaporated cautiously but not until complete dryness. Yield (theoretically 1.26 g) was assumed to be almost quantitative based on NMR and analytical HPLC.

¹H NMR (400 MHz, CDCl₃) δ 7.29 (m, 2H), 4.46 (s, 2H)

### (vi) 4-Aminomethyl-2,6-difluorobenzonitrile

This reaction was carried out according to the procedure described in J. Chem. Res. (M) (1992) 3128. To a suspension of 520 mg of 10% Pd/C (50% moisture) in 20 mL of water was added a solution of sodium borohydride (0.834 g, 0.0221 mol) in 20 mL of water. Some gas evolution resulted. 4-Azidomethyl-2,6-difluorobenzonitrile (1.26 g, 6.49 mmol; see step (v) above) was dissolved in 50 mL of THF and added to the aqueous mixture on an ice bath over 15 min. The mixture was stirred for 4 h, whereafter 20 mL of 2M HCl was added and the mixture was filtered through Celite. The Celite was rinsed with more water and the combined aqueous phase was washed with EtOAc and subsequently made alkaline with 2M NaOH. Extraction three times with methylene chloride followed and the combined organic phase was washed with water, dried (Na₂SO₄) and evaporated. Yield: 0.87 g (80%).

¹H NMR (400 MHz, CDCl₃) δ 7.20 (m, 2H), 3.96 (s, 2H), 1.51 (broad, 2H)

### (vii) 2,6-Difluoro-4-tert-butoxycarbonylaminomethylbenzonitrile

A solution of 4-aminomethyl-2,6-difluorobenzonitrile (0.876 g, 5.21 mmol; see step (vi) above) was dissolved in 50 mL of THF and di-tert-butyl dicarbonate (1.14 g, 5.22 mmol) in 10 mL of THF was added. The mixture was stirred for 3.5 h. The THF was evaporated and the residue was partitioned between water and EtOAc. The organic layer was washed three times with 0.5 M HCl and water, dried (Na₂SO₄) and evaporated. The product could be used without further purification. Yield: 1.38 g (99%).

¹H NMR (300 MHz, CDCl₃) δ 7.21 (m,2H), 4.95 (broad, 1H), 4.43 (broad, 2H), 1.52 (s, 9H)

### (viii) Boc-Pab(2,6-diF)(OH)

A mixture of 2,6-difluoro-4-tert-butoxycarbonylaminomethylbenzonitrile (1.38 g, 5.16 mmol; see step (vii) above), hydroxylamine hydrochloride (1.08 g, 0.0155 mol) and triethylamine (1.57 g, 0.0155 mol) in 20 mL of ethanol was stirred at room temperature for 36 h. The solvent was evaporated and the residue was partitioned between water and methylene chloride. The organic layer was washed with water, dried (Na₂SO₄) and evaporated. The product could be used without further purification. Yield: 1.43 g (92%).

¹H NMR (500 MHz, CD₃OD) δ 7.14 (m, 2H), 4.97 (broad, 1H), 4.84 (broad, 2H), 4.40 (broad, 2H), 1.43 (s, 9H)

### (ix) Boc-Pab(2,6-diF)×HOAc

This reaction was carried out according to the procedure described by Judkins et al, Synth. Comm. (1998) 4351. Boc-Pab(2,6-diF)(OH) (1.32 g, 4.37 mmol; see step (viii) above), acetic anhydride (0.477 g, 4.68 mmol) and 442 mg of 10% Pd/C (50% moisture) in 100 mL of acetic acid was hydrogenated at 5 atm pressure for 3.5 h. The mixture was filtered through Celite, rinsed with ethanol and evaporated. The residue was freeze-dried from acetonitrile and water and a few drops of ethanol. The sub-title product could be used without further purification. Yield: 0.1.49 g (99%).

¹H NMR (400 MHz, CD₃OD) δ 7.45 (m, 2H), 4.34 (s, 2H), 1.90 (s, 3H), 1.40 (s, 9H)

### (x) Boc-Pab(2,6-diF)(Teoc)

To a solution of Boc-Pab(2,6-diF)×HOAc (1.56 g, 5.49 mmol; see step (ix) above) in 100 mL of THF and 1 mL of water was added 2-(trimethylsilyl)ethyl p-nitrophenyl carbonate (1.67 g, 5.89 mmol). A solution of potassium carbonate (1.57 g, 0.0114 mol) in 20 mL of water was added dropwise over 5 min. The mixture was stirred overnight. The THF was evaporated and the residue was partitioned between water and methylene chloride. The aqueous layer was extracted with methylene chloride and the combined organic phase was washed twice with aqueous sodium bicarbonate, dried (Na₂SO₄) and evaporated. Flash chromatography on silica gel with heptane/EtOAc=2/1 gave 1.71 g (73%) of pure compound.

¹H NMR (400 MHz, CDCl₃) δ 7.43 (m, 2H), 4.97 (broad, 1H), 4.41 (broad, 2H), 4.24 (m, 2H), 1.41 (s, 9H), 1.11(m, 2H), 0.06 (s, 9H)

### (xi) Boc-Aze-Pab(2,6-diF)(Teoc)

Boc-Pab(2,6-diF)(Teoc) (1.009 g, 2.35 mmol; see step (x) above) was dissolved in 50 mL of EtOAc saturated with HCl(g). The mixture was left for 10 min., evaporated and dissolved in 18 mL of DMF, and then cooled on an ice bath. Boc-Aze-OH (0.450 g, 2.24 mmol), PyBOP (1.24 g, 2.35 mmol) and lastly diisopropylethyl amine (1.158 g, 8.96 mmol) were added. The reaction mixture was stirred for 2 h and then poured into 350 mL of water and extracted three times with EtOAc. The combined organic phase was washed with brine, dried (Na₂SO₄) and evaporated. Flash chromatography on silica gel with heptane:EtOAc (1:3) gave 1.097 g (96%) of the desired compound.

¹H NMR (500 MHz, CDCl₃) δ 7.46 (m, 2H), 4.65-4.5 (m, 3H), 4.23 (m, 2H), 3.87 (m, 1H), 3.74 (m, 1H), 2.45-2.3 (m, 2H), 1.40 (s, 9H), 1.10 (m, 2H), 0.05 (s, 9H)

### (xii) Ph(3-Cl)(5-OCHF₂)—(R)CH(OH)C(O)-Aze-Pab(2,6-diF)(Teoc)

Boc-Aze-Pab(2,6-diF)(Teoc) (0.256 g, 0.500 mmol; see step (xi) above) was dissolved in 20 mL of EtOAc saturated with HCl(g). The mixture was left for 10 min. and evaporated and dissolved in 5 mL of DMF. Ph(3-Cl)(5-OCHF₂)—(R)CH(OH)C(O)OH (0.120 g, 0.475 mmol; see Example 1(viii) above), PyBOP (0.263 g, 0.498 mmol) and lastly diisopropylethyl amine (0.245 g, 1.89 mmol were added. The reaction mixture was stirred for 2 h and then poured into 350 mL of water and extracted three times with EtOAc. The combined organic phase was washed with brine, dried (Na₂SO₄) and evaporated. Flash chromatography on silica gel with EtOAc gave 0.184 g (60%) of the desired sub-title compound.

¹H NMR (400 MHz, CD₃OD, mixture of rotamers) δ 7.55-7.45 (m, 2H), 7.32 (m, 1H, major rotamer), 7.27 (m, 1H, minor rotamer), 7.2-7.1 (m, 2H), 6.90 (t, 1H, major rotamer), 6.86 (t, 1H, minor rotamer), 5.15 (s, 1H, major rotamer), 5.12 (m, 1H, minor rotamer), 5.06 (s, 1H, minor rotamer), 4.72 (m, 1H, major rotamer), 4.6-4.45 (m, 2H), 4.30 (m, 1H, major rotamer), 4.24 (m, 2H), 4.13 (m, 1H, major rotamer), 4.04 (m, 1H, minor rotamer), 3.95 (m, 1H, minor rotamer), 2.62 (m, 1H, minor rotamer), 2.48 (m, 1H, major rotamer), 2.22 (m, 1H, major rotamer), 2.10 (m, 1H, minor rotamer), 1.07 (m, 2H), 0.07 (m, 9H)

### (xiii) Ph(3-Cl)(5-OCHF₂)—(R)CH(OH)C(O)-Aze-Pab(2,6-diF)

Ph(3-Cl)(5-OCHF₂)—(R)CH(OH)C(O)-Aze-Pab(2,6-diF)(Teoc) (81 mg, 0.127 mmol; see step (xii) above) was dissolved in 0.5 mL of methylene chloride and cooled on an ice bath. TFA (3 mL) was added and the reaction was left for 75 min. The TFA was evaporated and the residue was freeze dried from water and acetonitrile. The crude product was purified by preparative RPLC with CH₃CN:0.1M NH₄OAc (35:65) to produce 39 mg (55%) of the title compound as its HOAc salt, purity: 99%.

¹H NMR (400 MHz, CD₃OD mixture of rotamers) δ 7.5-7.4 (m, 2H), 7.32 (m, 1H, major rotamer), 7.28 (m, 1H, minor rotamer), 7.2-7.1 (m, 3H) 6.90 (t, 1H, major rotamer), 6.86 (t, minor rotamer), 5.15 (s, 1H, major rotamer), 5.14 (m, 1H, minor rotamer), 5.07 (s, 1H, minor rotamer), 4.72 (m, 1H, major rotamer), 4.65-4.45 (m, 2H), 4.30 (m, 1H, major rotamer), 4.16 (m, 1H, major rotamer), 4.03 (m, 1H, minor rotamer), 3.95 (m, 1H, minor rotamer), 2.63 (m, 1H, minor rotamer), 2.48 (m, 1H, major rotamer), 2.21 (m, 1H, major rotamer), 2.07 (m, 1H, minor rotamer), 1.89 (s, 3H)

¹³C-NMR (75 MHz; CD₃OD): (carbonyl and/or amidine carbons, mixture of rotamers) δ 171.9, 171.2, 165.0, 162.8, 160.4

APCI-MS: (M+1)=503/505 m/z.

### EXAMPLE 40

### Ph(3-Cl)(5-OCHF₂)—(R)CH(OH)C(O)-Aze-Pab(2,6-diF)(OMe)

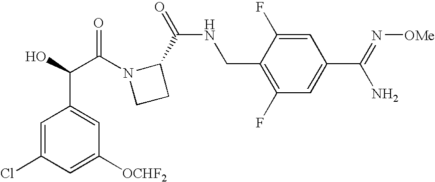

### (i) Ph(3-Cl)(5-OCHF₂)—(R)CH(OH)C(O)-Aze-Pab(2,6-diF)(OMe,Teoc)

A mixture of Ph(3-Cl)(5-OCHF₂)—(R)CH(OH)C(O)-Aze-Pab(2,6-diF)(Teoc) (64 mg, 0.099 mmol; see Example 39(xii) above) and O-methyl hydroxylamine hydrochloride (50 mg, 0.60 mmol) in 4 mL of acetonitrile was heated at 70° C. for 3 h. The solvent was evaporated and the residue was partitioned between water and EtOAc. The aqueous layer was extracted twice with EtOAc and the combined organic phase was washed with water, dried (Na₂SO₄) and evaporated. The product could be used without further purification. Yield: 58 mg (87%).

¹H NMR (400 MHz, CDCl₃) δ 7.90 (bt, 1H), 7.46 (m, 1H), 7.25-6.95 (m, 5H), 6.51, t, 1H), 4.88 (s, 1H), 4.83 (m, 1H), 4.6-4.5 (m, 2H), 4.4-3.9 (m, 4H), 3.95 (s, 3H), 3.63 (m, 1H), 2.67 (m, 1H), 2.38 (m, 1H), 1.87 (broad, 1H), 0.98 (m, 2H), 0.01, s, 9H)

### (ii) Ph(3-Cl)(5-OCHF₂)—(R)CH(OH)C(O)-Aze-Pab(2,6-diF)(OMe)

Ph(3-Cl)(5-OCHF₂)—(R)CH(OH)C(O)-Aze-Pab(2,6-diF)(OMe,Teoc) (58 mg, 0.086 mmol; see step (i) above) was dissolved in 3 mL of TFA, cooled on an ice bath and allowed to react for 2 h. The TFA was evaporated and the residue dissolved in EtOAc. The organic layer was washed twice with aqueous sodium carbonate and water, dried (Na₂SO₄) and evaporated. The residue was freeze-dried from water and acetonitrile to give 42 mg (92%) of the title compound. Purity: 94%.

¹H NMR (300 MHz, CDCl₃) δ 7.95 (bt, 1H), 7.2-7.1 (m, 4H), 6.99 (m, 1H), 6.52 (t, 1H), 4.88 (s, 1H), 4.85-4.75 (m, 3H), 4.6-4.45 (m, 2H), 4.29 (broad, 1H), 4.09 (m, 1H), 3.89 (s, 3H), 3.69 (m, 1H), 2.64 (m, 1H), 2.38 (m, 1H), 1.85 (broad, 1H)

¹³C-NMR (100 MHz; CDCl₃): (carbonyl and/or amidine carbons) δ 172.1, 169.8, 151.9

APCI-MS: (M+1)=533/535 m/z

### EXAMPLE 41

### Ph(3-Cl)(5-OCHF₂)—(R)CH(OH)C(O)-Aze-Pab(2,5-diF)

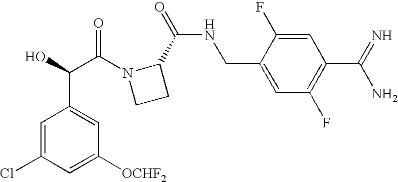

### (i) 2,5-Difluoro-4[(methylsulfinyl)(methylthio)methyl]benzonitrile

(Methylsulfinyl)(methylthio)methane (3.16 g, 0.0255 mol) was dissolved in 50 mL of dry THF under argon and then cooled to −78° C. Butyllithium in hexane (16 mL 1.6M, 0.0256 mol) was added dropwise with stirring. The mixture was stirred for 15 min. Meanwhile a solution of 2,4,5-trifluorobenzonitrile (2.0 g; 0.013 mol) in 50 mL of dry THF was cooled to −78° C. under argon and the former solution was added through a cannula to the latter solution over a period of 3-5 min. After 30 min, the cooling bath was removed and when the reaction had reached room temperature it was poured into 200 mL of water. The THF was evaporated and the remaining aqueous layer was extracted three times with diethyl ether. The combined ether phase was washed with water, dried (Na₂SO₄) and evaporated. The crude product started to crystallise and could be used as such in the next step. Yield: 2.8 g (84%).

¹H NMR (500 MHz, CDCl₃) δ 7.51-7.44 (m, 2H, major diastereomer), 7.39 (dd, 1H, minor diastereomer), 5.00 (s, 1H, minor diastereomer), 4.92 (s, 1H, major diastereomer), 2.59 (s, 3H, minor diastereomer), 2.56 (s, 1H, major diastereomer), 2.46 (s, 1H, minor diastereomer), 2.40 (s, 1H, major diastereomer)

### (ii) 2,5-Difluoro-4-formylbenzonitrile

2,5-Difluoro-4[(methylsulfinyl)(methylthio)methyl]benzonitrile (2.8 g, 0.0107 mol; see step (i) above) was dissolved in 100 mL of THF and 6.5 g of concentrated sulfuric acid was added. The mixture was left at room temperature for 6 days and subsequently poured into 500 mL of water. Extraction three times with diethyl ether followed and the combined ethereal phase was washed several times with water, dried (Na₂SO₄) and evaporated. The crude product was flash chromatographed on silica gel using heptane:EtOAc (8:2). Yield: 1.2 g (67%). The position of the formyl group was established by use of ¹³C NMR. The carbon signals from the fluorinated carbons at 160.1 and 158.4 respectively were doublets and not quartets, which they would have been if the formyl group had been in the 2-position.

¹H NMR (300 MHz, CDCl₃) δ 10.36 (d, 1H), 7.72 (dd, 1H), 7.54 (dd, 1H)

### (iii) 2,5-Difluoro-4-hydroxymethylbenzonitrile

2,5-Difluoro-4-formylbenzonitrile (3.60 g, 0.0215 mol; see step (ii) above) was dissolved in 50 mL of methanol and cooled on an ice bath. Sodium borohydride (0.815 g, 0.0215 mol) was added in portions with stirring and the reaction was left for 45 min. Water (300 mL) was added and thereafter carefully 2M HCl was added until an acidic pH was attained. The mixture was extracted three times with diethyl ether, and the combined ethereal phase was washed with water, dried (Na₂SO₄) and evaporated. The crude product crystallised soon and could be used without further purification. Yield: 3.1 g (85%).

¹H NMR (300 MHz, CDCl₃) δ 7.45 (dd, 1H), 7.30 (dd, 1H), 4.85 (s, 2H), 2.10 (broad, 1H)

### (iv) 4-Cyano-2,5-difluorobenzyl methanesulfonate

To an ice cooled solution of 2,5-difluoro-4-hydroxymethylbenzonitrile (3.10 g, 0.0183 mol; see step (iii) above) and methanesulfonyl chloride (2.21 g, 0.0192 mol) in 60 mL of methylene chloride was added triethyl amine (1.95 g, 0.0192 mol) with stirring. After 1.5 h at 0° C. the mixture was washed with water, dried (Na₂SO₄) and evaporated. The product could be used without further purification. Yield: 4.5 g (99%).

¹H NMR (300 MHz, CDCl₃) δ 7.45-7.35 (m, 2H), 5.32 (s, 2H), 3.13 (s, 3H)

### (v) 4-Azidomethyl-2,5-difluorobenzonitrile

A mixture of 4cyano-2,5-difluorobenzyl methanesulfonate (4.5 g, 0.0182 mol; see step (iv) above) and sodium azide (2.0 g, 0.031 mol) in 20 mL of water and 40 mL of DMF was stirred at room temperature for 2 h. It was subsequently poured into 300 mL of water and extracted three times with diethyl ether. The combined ethereal phase was washed several times with water, dried (Na₂SO₄) and evaporated. A small sample was evaporated for NMR purposes and the product crystallised. The rest was evaporated cautiously but not until complete dryness. Yield (theoretically 3.5 g) is assumed to be almost quantitative based on NMR and analytical HPLC.

¹H NMR (500 MHz, CDCl₃) δ 0.38 (dd, 1H), 7.32 (dd, 1H), 4.54 (s, 2H)

### (vi) 4-Aminomethyl-2,5-difluorobenzonitrile

This reaction was carried out according to the procedure described in J. Chem. Res. (M) (1992) 3128. To a suspension of 300 mg of 10% Pd/C (50% moisture) in 20 mL of water was added a solution of sodium borohydride (0.779 g, 0.0206 mol) in 20 mL of water. Some gas evolution resulted. 4-Azidomethyl-2,5-difluorobenzonitrile (1.00 g, 5.15 mmol; from step (v) above) was dissolved in 60 mL of THF and added to the aqueous mixture on an ice bath. The mixture was stirred for 1.5 h whereafter 10 mL of 2M HCl was added and the mixture was filtered through Celite. The Celite was rinsed with more water and the combined aqueous phase was washed with EtOAc and subsequently made alkaline with 2M NaOH. Extraction three times with methylene chloride followed and the combined organic phase was washed with water, dried (Na₂SO₄) and evaporated. Yield: 0.47 g (54%).

¹H NMR (300 MHz, CDCl₃) δ 7.39 (dd, 1H), 7.29 (dd, 1H), 3.99 (s, 2H), 1.45 (broad, 2H)

### (vii) 2,5-Difluoro-4-tert-butoxycarbonylaminomethylbenzonitrile

A solution of 4-aminomethyl-2,5-difluorobenzonitrile (0.46 g, 2.7 mmol; see step (vi) above) and di-tert-butyl dicarbonate (0.60 g, 2.7 mmol) in 10 mL of THF was stirred overnight. The THF was evaporated and the residue was partitioned between water and EtOAc. The organic layer was washed with water, dried (Na₂SO₄) and evaporated. The product could be used without further purification. Yield: 0.71 g (97%).

¹H NMR (300 MHz, CDCl₃) δ 7.35-7.2 (m, 2H), 5.11 (broad triplet, 1H), 4.38 (d, 2H), 1.45 (s, 9H)

### (viii) Boc-Pab(2,5-diF)(OH)

A mixture of 2,5-difluoro-4-tert-butoxycarbonylaminomethylbenzonitrile (0.70 g, 2.6 mmol; see step (vii) above), hydroxylamine hydrochloride (0.54 g, 7.8 mmol) and triethylamine (0.79 g, 7.8 mmol) in 10 mL of ethanol was stirred at room temperature for 6 days. It was then partitioned between water and methylene chloride. The aqueous layer was extracted with methylene chloride and the combined organic phase was washed with water, dried (Na₂SO₄) and evaporated. The product could be used without further purification. Yield: 0.72 g (92%).

¹H NMR (500 MHz, CD₃OD) δ 7.27 (dd, 1H), 7.12 (dd, 1H), 4.29 (s, 2H), 1.47 (s, 9H)

### (ix) Boc-Pab(2,5-diF)×HOAc

This reaction was carried out according to the procedure described by Judkins et al, Synth. Comm. (1998) 4351. Boc-Pab(2,5-diF)(OH) (0.70 g, 2.3 mmol; see step (viii) above), acetic anhydride (0.25 g, 2.4 mmol) and 230 mg of 10% Pd/C (50% moisture) in 70 mL of acetic acid was hydrogenated at 5 atm pressure for 2.5 h. The mixture was filtered through Celite and evaporated. The residue was freeze dried from acetonitrile and water. The product could be used without further purification in the next step. Yield: 0.80 g (100%).

¹H NMR (500 MHz, CD₃OD) δ 7.49 (dd, 1H), 7.31 (dd, 1H), 4.33 (s, 2H), 1.91 (s, 3H), 1.46 (s, 9H)

### (x) Boc-Pab(2,5-diF)(Teoc)

To a suspension of Boc-Pab(2,5-diF)×HOAc (0.80 g, 2.3 mmol; see step (ix) above) in 50 mL of THF was added 2-(trimethylsilyl)ethyl p-nitrophenyl carbonate (0.85 g, 3.0 mmol). A solution of potassium carbonate (0.80 g, 5.8 mmol) in 10 mL of water was added dropwise. The mixture was stirred overnight. The excess Teoc reagent was destroyed by addition of glycine (0.100 g) and potassium carbonate (0.75 g) to the solution, letting it react for an additional 2 h. The THF was evaporated and the residue was partitioned between water and methylene chloride. The aqueous layer was extracted with methylene chloride and the combined organic phase was washed with water, dried (Na₂SO₄) and evaporated. Flash chromatography on silica gel with heptane:EtOAc (2: 1) gave 0.72 g (72%) of pure compound.

¹H NMR (400 MHz, CDCl₃) δ 8.00 (dd, 1H), 7.15 (dd, 1H), 4.98 (broad, 1H), 4.36 (bd, 2H), 4.24 (m, 2H), 1.45 (s, 9H), 1.12 (m, 2H), 0.07 (s, 9H)

### (xi) H-Pab(2,5-diF)(Teoc)×2 HCl

Boc-Pab(2,5-diF)(Teoc) (0.38 g, 0.88 mmol; see step (x) above) was dissolved in 50 mL of EtOAc saturated with HCl(g). The mixture was left for 30 min and evaporated.

¹H NMR (500 MHz, CD₃OD) δ 7.75-7.6 (m, 2H), 4.46 (m, 2H), 4.3 (s, 2H), 1.15 (m, 2H), 0.07 (s, 9H)

### (xii) Boc-Aze-Pab(2,5-diF)(Teoc)

To a stirred solution of Boc-Aze-OH (0.189 g, 0.94 mmol), H-Pab(2,5-diF)(Teoc)×2 HCl (0.36 g, 0.89 mmol; see step (xi) above) and PyBOP (0.54 g, 1.03 mmol) in 5 mL of DMF was added diisopropylethyl amine (0.49 g, 3.8 mmol) and the mixture was allowed to react overnight. The resultant was then poured into aqueous sodium bicarbonate and extracted three times with EtOAc. The combined organic phase was washed with water, dried (Na₂SO₄) and evaporated. Flash chromatography on silica gel with heptane:EtOAc (3:7) gave a sufficiently pure compound. Yield: 0.25 g (48%).

¹H NMR (500 MHz, CDCl₃) δ 7.98 (dd, 1H), 7.13 (dd, 1H), 4.69 (m, 1H), 4.53 (m, 2H), 4.22 (m, 2H), 3.92 (m, 1H), 3.79 (m, 1H), 2.55-2.35 (m, 2H), 1.44 (s, 9H), 1.11 (m, 2H), 0.06 (s, 9H)

### (xiii) H-Aze-Pab(2,5-diF)(Teoc)×2 HCl

Boc-Aze-Pab(2,5-diF)(Teoc) (0.25 g, 0.49 mmol; see step (xii) above) was dissolved in 50 mL of EtOAc saturated with HCl(g). The mixture was left for 30 min. and evaporated. The product was used in the next step without further purification. Yield: 0.23 g (97%).

¹H NMR (400 MHz, CD₃OD) δ 7.59 (dd, 1H), 7.47 (dd, 1H), 5.14 (m, 1H), 4.54 (m, 2H), 4.48 (m,2H), 4.15 (m, 1H), 3.96 (m, 1H), 2.87 (m, 1H), 2.56 (m, 1H), 1.17 (m, 2H), 0.05 (s, 9H)

### (xiv) Ph(3-Cl)(5-OCHF₂)—(R)CH(OH)C(O)-Aze-Pab(2,5-diF)(Teoc)

To a solution of Ph(3-Cl)(5-OCHF₂)—(R)CH(OH)C(O)OH (0.12 g, 0.47 mmol; see Example 1(viii) above), H-Aze-Pab(2,5-diF)(Teoc)×2 HCl (0.23 g, 0.47 mmol; see step (xiii) above) and PyBOP (0.27 g, 0.52 mmol) in 10 mL of DMF was added diisopropylethyl amine (0.245 g, 1.90 mmol), and the mixture was stirred overnight. The resultant was poured into water and extracted three times with EtOAc. The combined organic phase was washed with water, dried (Na₂SO₄) and evaporated. Flash chromatography on silica gel with EtOAc gave 100 mg of a pure fraction and 30 mg of a 90% pure fraction. Total yield: 0.13 g (41%).

¹H NMR (400 MHz, CDCl₃) δ 9.80 (broad, 1H), 8.05 (bt, 1H), 7.94 (dd, 1H), 7.20 (m, 1H), 7.2-7.1 (m, 2H), 7.02 (m, 1H), 6.54 (t, 1H), 4.93 (s, 1H), 4.91 (m, 1H), 4.51 (m, 2H), 4.28 (broad, 1H), 4.23 (m, 2H), 4.13 (m, 1H), 3.74 (m, 1H), 2.69 (m, 1H), 2.43 (m, 1H), 1.73 (broad, 1H), 1.11 (m, 2H), 1.11 (s, 9H)

### (xv) Ph(3-Cl)(5-OCHF₂)—(R)CH(OH)C(O)-Aze-Pab(2,5-diF)

Ph(3-Cl)(5-OCHF₂)—(R)CH(OH)C(O)-Aze-Pab(2,5-diF)(Teoc) (60 mg (0.093 mmol) of the pure fraction from step (xiv) above) was dissolved in 3 mL of TFA and left at room temperature for 1 h. The TFA was evaporated and the residue was freeze-dried from water and acetonitrile to produce 55 mg (96%) of the title compound as its TFA salt, purity: >99%.

¹H NMR (500 MHz, CD₃OD mixture of rotamers) δ 7.55-7.3 (m, 3H), 7.2-7.1 (m, 2H), 6.88 (t, 1H, major rotamer), 6.86 (t, 1H, minor rotamer), 5.22 (m, 1H, minor rotamer), 5.20 (s, 1H, major rotamer), 5.13 (s, 1H, minor rotamer), 4.80 (m, 1H, major rotamer), 4.6-4.45 (m, 2H), 4.36 (m, 1H, major rotamer), 4.19 (m, 1H, major rotamer), 4.07 (m, 1H, minor rotamer), 3.98 (m, 1H, minor rotamer), 2.70 (m, 1H, minor rotamer), 2.54 (m, 1H, major rotamer), 2.28 (m, 1H, major rotamer), 2.14 (m, 1H, minor rotamer)

¹³C-NMR (75 MHz; CD₃OD): (carbonyl and/or amidine carbons, mixture of rotamers) δ 173.0, 172.6, 172.1, 172.0, 162.4

APCI-MS: (M+1)=503/505 m/z.

### EXAMPLE 42

### Ph(3-Cl)(5-OCHF₂)—(R)CH(OH)C(O)-Aze-Pab(2,5-diF)(OMe)

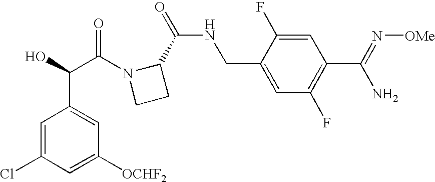

### (i) Ph(3-Cl)(5-OCHF₂)—(R)CH(OH)C(O)-Aze Pab(2,5-diF)(OMe,Teoc)

A mixture of Ph(3-Cl)(5-OCHF₂)—(R)CH(OH)C(O)-Aze-Pab(2,5-diF)(Teoc) (40 mg, 0.062 mmol; see Example 41(xiv) above) and O-methyl hydroxylamine hydrochloride (58 mg, 0.70 mmol) in 5 mL of acetonitrile was heated at 70° C. for 2 h. The solvent was evaporated and the residue was partitioned between water and EtOAc. The aqueous layer was extracted with EtOAc and the combined organic phase was washed with water, dried (Na₂SO₄) and evaporated. The product could be used without further purification. Yield: 35 mg (84%).

¹H NMR (600 MHz, CDCl₃) δ 7.99 (bt, 1H), 7.72 (s, 1H), 7.20 (m, 1H) 7.15-7.1 (m, 1H), 7.07 (dd, 1H), 7.01 (m, 1H), 6.53 (t, 1H), 4.90 (s, 1H), 4.88 m, 1H), 4.48 (m, 2H), 4.2-4.1 (m, 3H), 3.95 (s, 3H), 3.67 (m, 1H), 2.68 (m, 1H), 2.41 (m, 1H), 0.97 (m, 2H), 0.07 (s, 9H)

### (ii) Ph(3-Cl)(5-OCHF₂)—(R)CH(OH)C(O)-Aze-Pab(2,5-diF)(OMe)

Ph(3-Cl)(5-OCHF₂)—(R)CH(OH)C(O)-Aze-Pab(2,5-diF)(OMe,Teoc) (35 mg, 0.052 mmol; see step (i) above) was dissolved in 3 mL of TFA and allowed to react for 30 min. The TFA was evaporated and the residue freeze-dried from water and acetonitrile to give 29 mg (99%) of the title compound. Purity: 97%.

¹H NMR (300 MHz, CDCl₃) δ 8.01 (bt, 1H), 7.45 (dd, 1H), 7.20 (m, 1H), 7.15 (m, 1H), 7.09 (dd, 1H), 7.02 (m, 1H), 6.54 (t, 1H), 5.2-5.0 (m, 2H), 4.95-4.85 (m, 2H), 4.6-4.4 (m, 2H), 4.25 (broad, 1H), 4.13 (m, 1H), 3.90 (s, 3H), 3.71 (m, 1H), 2.69 (m, 1H), 2.43 (m, 1H)

¹³C-NMR (75 MHz; CDCl₃): (carbonyl and/or amidine carbons) δ 173.0, 170.9, 152.6

APCI-MS: (M+1)=533/535 m/z.

### EXAMPLE 43

### Ph(3-Cl)(5-OCHF₂)—(R)CH(OH)C(O)-Aze-Pab(OEt)

### (i) Ph(3-Cl)(5-OCHF₂)—(R)CH(OH)C(O)-Aze-Pab(OEt, Teoc)

Ph(3-Cl)(5-OCHF₂)—(R)CH(OH)C(O)-Aze-Pab(Teoc) (55 mg, 0.090 mmol; see Example 1(ix) above) and O-ethylhydroxyl amine hydrochloride (53 mg, 0.54 mmol) were dissolved in 4 mL of THF. The mixture was stirred at 60° C. for 5 h. The solvent was evaporated. The residue was chromatographed on silica gel, eluting with methylene chloride:methanol (95:5) to afford 55 mg (93%) of the sub-title compound.

¹H-NMR (400 MHz; CDCl₃): δ 7.84 (bt, 1H), 7.59 (bs, 1H), 7.47 (bd, 1H), 7.29 (bd, 1H), 7.21 (m, 1H), 7.14 (m, 1H), 7.02 (m, 1H), 6.53 (t, 1H), 4.90 (s, 1H), 4.86 (m, 1H), 4.55-4.4 (m, 2H), 4.25-4.1 (m, 5H), 3.69 (m, 1H), 2.66 (m, 1H), 2.41 (m, 1H), 1.33 (t, 3H), 0.98 (m, 2H), 0.02 (s, 9H)

### (ii) Ph(3-Cl, 5-OCHF₂)—(R)CH(OH)C(O)-Aze-Pab(OEt)

To an ice-cold solution of Ph(3-Cl)(5-OCHF₂)—(R)CH(OH)C(O)-Aze-Pab(OEt, Teoc) (55 mg, 0.084 mmol; see step (i) above) in 0.5 mL of methylene chloride was added 3 mL of TFA. The mixture was stirred (ice-bath) for 160 minutes. The material was purified using preparative HPLC. The fractions of interest were pooled and freeze-dried (2×), yielding 20 mg (47%) of the title compound.

¹H-NMR (400 MHz; CD₃OD) rotamers: δ 7.59 (bd, 2H), 7.35 (m, 1H), 7.32 (bd, 2H), 7.25-7.1 (m, 2H), 6.89 (t, 1H, major rotamer), 6.86 (t, 1H, minor rotamer), 5.18 (s, 1H, major rotamer), 5.18 (m, 1H, minor rotamer), 5.11 (s, 1H, minor rotamer), 4.77 (m, 1H), 4.5-4.3 (m, 3H), 4.2-3.9 (m, 3H), 2.67 (m, 1H, minor rotamer), 2.52 (m, 1H, major rotamer), 2.28 (m, 1H, major rotamer), 2.15 (m, 1H, minor rotamer), 1.28 (t, 3H)

¹³C-NMR (100 MHz; CD₃OD): (carbonyl and/or amidine carbons, rotamers) δ 172.4, 171.9, 171.4, 153.8, 152.3

MS (m/z) 509 (M−1)⁻, 511 (M+1)⁺

### EXAMPLE 44

### Ph(3-Cl)(5-OCHF₂)—(R)CH(OH)C(O)-Aze-Pab(OnPr)

### (i) Ph(3-Cl)(5-OCHF₂)—(R)CH(OH)C(O)-Aze-Pab(OnPr, Teoc)

Ph(3-Cl)(5-OCHF₂)—(R)CH(OH)C(O)-Aze-Pab(Teoc) (53 mg, 0.087 mmol; see Example 1(ix) above) and O-n-propylhydroxyl amine hydrochloride, 58 mg (0.52 mmol) were dissolved in 4 mL of THF. The mixture was stirred at 60° C. for 5 h. The solvent was evaporated. The residue was chromatographed on silica gel, eluting with methylene chloride:methanol (95:5) to afford 51 mg (88%) of the sub-title compound.

¹H-NMR (400 MHz; CDCl₃): δ 7.84 (m, 1H), 7.59 (bs, 1H), 7.47 (bd, 2H), 7.28 (bd, 2H), 7.21 (m, 1H), 7.14 (m, 1H), 7.02 (m, 1H), 6.53 (t, 1H), 4.90 (s, 1H), 4.85 (m, 1H), 4.55-4.4 (m, 2H), 4.2-4.05 (m, 5H), 3.69 (m, 1H), 2.65 (m, 1H), 2.41 (m, 1H), 1.74 (m, 2H), 1.05-0.95 (m, 5H), 0.03 (s, 9H)

### (ii) Ph(3-Cl)(5-OCHF₂)—(R)CH(OH)C(O)-Aze-Pab(OnPr)

To an ice-cold solution of Ph(3-Cl)(5-OCHF₂)—(R)CH(OH)C(O)-Aze-Pab(OnPr, Teoc) (51 mg, 0.078 mmol; see step (i) above) in 0.5 mL of methylene chloride was added 3 mL of TFA. The mixture was stirred (ice-bath) for 110 minutes. The material was purified using preparative HPLC. The fraction of interest was evaporated and freeze-dried, yielding 20 mg (47%) of the title compound.

¹H-NMR (500 MHz; CD₃OD) rotamers: δ 7.61 (bd, 2H), 7.38 (m, 1H), 7.35 (bd, 2H), 7.22 (m, 1H, major rotamer), 7.18 (m, 1H), 7.15 (m, 1H, minor rotamer), 6.92 (t, 1H, major rotamer), 6.89 (t, 1H, minor rotamer), 5.20 (s, 1H, major rotamer), 5.20 (m, 1H, minor rotamer), 4.80 (m, 1H, major rotamer), 4.5-4.4 (m, 2H, including minor rotamer corresponding to major at 4.37), 4.37 (m, 1H, major rotamer), 4.18 (m, 1H, major rotamer), 4.09 (m, 1H, minor rotamer), 3.99 (m, 2H), 2.70 (m, 1H, minor rotamer), 2.54 (m, 1H, major rotamer), 2.30 (m, 1H, major rotamer), 2.18 (m, 1H, minor rotamer), 1.73 (m, 2H), 1.01 (t, 3H)

¹³C-NMR (125 MHz; CD₃OD): (carbonyl and/or amidine carbons, rotamers) δ 171.4, 153.8, 152.3

MS (m/z) 523 (M−1)⁻, 525 (M+1)⁺

### EXAMPLE 45

### Ph(3-Cl)(5-OCHF₂)—(R)CH(OH)C(O)-Aze-Pab(OiPr)

### (i) Ph(3-Cl)(5-OCHF₂)—(R)CH(OH)C(O)-Aze-Pab(OiPr, Teoc)

Ph(3-Cl)(5-OCHF₂)—(R)CH(OH)C(O)-Aze-Pab(Teoc) (50 mg, 0.082 mmol; see Example 1(ix) above) and O-i-propylhydroxyl amine hydrochloride, 55 mg (0.49 mmol) were dissolved in 4 mL of THF. The mixture was stirred at 60° C. for 5 h. The solvent was evaporated. The residue was chromatographed on silica gel, eluting with methylene chloride:methanol (95:5) to afford 46 mg (84%) of the sub-title compound.

¹H-NMR (400 MHz; CDCl₃): δ 7.84 (m, 1H), 7.57 (bs, 1H), 7.48 (bd, 2H), 7.29 (bd, 2H), 7.21 (m, 1H), 7.14 (m, 1H), 7.02 (m, 1H), 6.53 (t, 1H), 4.91 (s, 1H), 4.87 (m, 1H), 4.55-4.45 (m, 2H), 4.42 (m, 1H), 4.2-4.1 (m, 3H), 3.69 (m, 1H), 2.66 (m, 1H), 2.42 (m, 1H), 1.30 (d, 6H), 0.98 (m, 2H), 0.02 (s, 9H)

### (ii) Ph(3-Cl)(5-OCHF₂)—(R)CH(OH)C(O)-Aze-Pab(OiPr)

To an ice-cold solution of Ph(3-Cl)(5-OCHF₂)—(R)CH(OH)C(O)-Aze-Pab(OiPr, Teoc) (46 mg, 0.069 mmol; see step (i) above) in 0.5 mL of methylene chloride was added 3 mL of TFA. The mixture was stirred (ice-bath) for 150 minutes. The material was purified using preparative HPLC. The fraction of interest was evaporated and freeze-dried (2×), yielding 22 mg (58%) of the title compound.

¹H-NMR (400 MHz; CD₃OD) rotamers: δ 7.59 (d, 2H), 7.35 (m, 1H), 7.32 (d, 2H), 7.19 (m, 1H, major rotamer), 7.15 (m, 1H), 7.12 (m, 1H, minor rotamer), 6.89 (t, 1H, major rotamer), 6.86 (t, 1H, minor rotamer), 5.18 (s, 1H, major rotamer), 5.18 (m, 1H, minor rotamer), 5.12 (s, 1H, minor rotamer), 4.78 (m, 1H, major rotamer), 4.5-3.9 (m, 5H), 2.67 (m, 1H, minor rotamer), 2.52 (m, 1H, major rotamer), 2.28 (m, 1H, major rotamer), 2.15 (m, 1H, minor rotamer), 1.26 (d, 6H)

¹³C-NMR (100 MHz; CD₃OD): (carbonyl and/or amidine carbons, rotamers) δ 171.9, 171.4, 153.6.

MS (m/z) 523 (M−1)⁻, 525 (M+1)⁺

### EXAMPLE 46

The title compounds of Examples 3, 6, 9, 10, 13 to 15, 17, 19, 21, 23, 25, 27, 28, 32, 34, 36, 38, 39 and 41 were tested in Test A above and were found to exhibit IC₅₀TT values of less than 3.5 μM. Those of Examples 3, 6, 9, 10, 13, 15, 17, 19, 21, 23, 27, 32, 34 and 39 were found to exhibit values of less than 0.02 μM; those of Examples 25 and 28 less than 0.03 μM, that of Example 14 less than 0.04 μM; and those of Examples 38 and 41 less than 0.15 μM.

### EXAMPLE 47

The title compounds of Examples 3, 6, 13, 15, 17, 19, 21, 23, 25, 27, 28, 32 and 34 were tested in Test D above and were found to exhibit an IC₅₀ APTT value of less than 1 TM.

### EXAMPLE 48

The title compounds of Examples 1, 2, 4, 5, 7, 12, 16, 18, 20, 22, 24, 26, 29, 30, 33 and 43 to 45 were tested in Test E above and were found to exhibit oral and/or parenteral bioavailability in the rat as the corresponding active inhibitor (free amidine).

### EXAMPLE 49

Title compounds of Examples 1, 2, 7, 8, 11, 12, 16, 18, 20, 22, 24, 26, 29, 33, 37, 40, 43 and 45 were tested in Test G above and were found to be converted to the corresponding active inhibitor (free amidine) in liver microsomes from humans and from rats.

Abbreviations

Ac=acetyl
AcOH=acetic acid
APCI=atmospheric pressure chemical ionisation (in relation to MS)
API=atmospheric pressure ionisation (in relation to MS)
is aq.=aqueous
AUC=area under the curve
Aze=(S)-azetidine-2-carboxylate (unless otherwise specified)
AzeOH=azetidine-2-carboxylic acid
Bn=benzyl
Boc=tert-butyloxycarbonyl
BSA=bovine serum albumin
Bu=butyl
Bzl=benzyl
CI=chemical ionisation (in relation to MS)
d=day(s)
DCC=dicyclohexyl carbodiimide
DEBAL-H=di-isobutylaluminium hydride
DIPEA=diisopropylethylamine
DMAP=4-(NN-dimethyl amino) pyridine
DMF=dimethylformamide
DMSO=dimethylsulfoxide
DVT=deep vein thrombosis
EDC=1-(3-dimethylaminopropyl)-3-ethylcarbodiimide hydrochloride
e.e.=enantiomeric excess
Et=ethyl
ether=diethyl ether
EtOAc=ethyl acetate
EtOH=ethanol
Et₂O=diethyl ether
h=hour(s)
HATU=O-(azabenzotriazol-1-yl)-N,N,N′,N′-tetramethyluronium hexafluorophosphate
HBTU=[N,N,N′,N-tetramethyl-O-(benzotriazol-1-yl)uronium hexafluorophosphate]
HCl=hydrochloric acid, hydrogen chloride gas or hydrochloride salt (depending on context)
Hex=hexanes
HOAc=acetic acid
HPLC=high performance liquid chromatography
LC=liquid chromatography
Me=methyl
MEM=methoxyethoxymethyl
MeOH=methanol
min=minute(s)
MS=mass spectroscopy
MTBE=methyl tert-butyl ether
NADH=nicotinamide adenine dinucleotide, reduced form
NADPH=nicotinamide adenine dinucleotide phosphate, reduced form
NIH=National Institute of Health (US)
NIHU=National Institute of Health units
NMR=nuclear magnetic resonance
OAc=acetate
Pab=para-amidinobenzylamino
H-Pab=para-amidinobenzylamine
Ph=phenyl
Pr=propyl
Pro=(S)-prolinyl
PyBOP=(benzotriazol-1-yloxy)tripyrrolidinophosphonium hexafluorophosphate
QF=tetrabutylammonium fluoride
RedAl=sodium bis(2-methoxyethoxy)aluminium hydride
RPLC=reverse phase high performance liquid chromatography
rt/RT=room temperature
SOPs=standard operating procedures
TBTU=[N,N,N′,N′-tetramethyl-O-(benzotriazol-1-yl)uronium tetrafluoroborate]
TEA=triethylamine
Teoc=2-(trimethylsilyl)ethoxycarbonyl
TEMPO=2,2,6,6-tetramethyl-1-piperidinyloxy free radical
TFA=trifluoroacetic acid
THF=tetrahydrofuran
THP=tetrahydropyranyl
TLC=thin layer chromatography
TMSCl=trimethylsilyl chloride
TMSCN=trimethylsilyl cyanide
UV=ultraviolet
Z=benzyloxycarbonyl

Prefixes n, s, i and t have their usual meanings: normal, secondary, iso and tertiary. The prefix c means cyclo.

1. A combination product comprising:
(a) a compound of claim 1 in WO 02/44145 or a pharmaceutically-acceptable derivative thereof; and
(b) (1) a compound as defined in claim 1 of WO 01/28992 or (2) a compound of claim 34 of WO 01/28992 or (3) Compound A or B or C or D (or pharmaceutically-acceptable salts thereof)
wherein each of components (a) and (b) is formulated in admixture with a pharmaceutically-acceptable adjuvant, diluent or carrier.

2. A combination product as claimed in claim 1 which comprises a pharmaceutical formulation including a compound of claim 1 in WO 02/44145 or a compound of claim 20 in WO 02/44145 or sub-set 1, 2 or 3 of the compounds of claim 20 or a pharmaceutically-acceptable derivative thereof, and (1) a compound as defined in claim 1 of WO 01/28992 or (2) a compound of claim 34 of WO 01/28992 or (3) Compound A or B or C or D (or pharmaceutically-acceptable salts thereof) or a pharmaceutically-acceptable derivative thereof, in admixture with a pharmaceutically-acceptable adjuvant, diluent or carrier.

3. A combination product as claimed in claim 1 which comprises a kit of parts comprising components:
(a) a pharmaceutical formulation including a compound of claim 1 in WO 02/44145 or a compound of claim 20 in WO 02144145 or sub-set 1,2 or 3 of the compounds of claim 20 or a pharmaceutically-acceptable derivative thereof, in admixture with a pharmaceutically-acceptable adjuvant, diluent or carrier; and
(b) a pharmaceutical formulation including (1) a compound as defined in claim 1 of WO 01/28992 or (2) a compound of claim 34 of WO 01/28992 or
(3) Compound A or B or C or D (or pharmaceutically-acceptable salts thereof), in admixture with a pharmaceutically-acceptable adjuvant, diluent or carrier, which components (a) and (b) are each provided in a form that is suitable for administration in conjunction with the other.

4. A kit of parts as claimed in claim 3, wherein components (a) and (b) are suitable for sequential, separate and/or simultaneous use in the treatment of a condition where anticoagulant therapy is indicated.

5. A combination product as claimed in any one of claims 1 to 4, which comprises Compound A or B or C or D (or pharmaceutically-acceptable salts thereof).

6. A combination product as claimed in any one of claims 1 to 5 wherein the compound of WO 02/44145 is selected from:
Ph(3-Cl)(5-OCHF₂)—(R)CH(OH)C(O)-Aze-Pab;
Ph(3-Cl)(5-OCF₃)—(R)CH(OH)C(O)-Aze-Pab;
Ph(3-Cl)(5-OCHF₂)—(S)CH(CH₂OH)C(O)-Aze-Pab;
Ph(3-Cl)(5-OCF₃)—(S)CH(CH₂OH)C(O)-Aze-Pab;
Ph(3-OCHF₂)—(R)CH(OH)-CO-Aze-Pab;
Ph(3-OCF₃)—(R)CH(OH)-CO-Aze-Pab;
Ph(3-Cl)(5-OCH₂CF₃)—(R)CH(OH)C(O)-Aze-Pab;
Ph(3-Cl)(5-OCH₂CHF₂)—(R)CH(OH)C(O)-Aze-Pab;
Ph(3-Cl)(5-OCH₂F)—(R)CH(OH)C(O)-Aze-Pab;
Ph(3-Cl)(5-OCH₂CH₂F)—(R)CH(OH)C(O)-Aze-Pab;
Ph(3-Cl)(5-OCH(CH₂F)₂)—(R)CH(OH)C(O)-Aze-Pab;
Ph(3-F)(5-OCHF₂)—(R)CH(OH)C(O)-Aze-Pab;
Ph(3-Br)(5-OCH₂F)—(R)CH(OH)C(O)-Aze-Pab;
Ph(3-Br)(5-OCHF₂)—(R)CH(OH)C(O)-Aze-Pab;
Ph(3-Cl, 5-OCHF₂)—(R)CH(OH)C(O)-Pro-Pab;
Ph(3-Cl, 5-OCHF₂)—(R)CH(OH)C(O)-Aze-NH—CH₂-((2-amidino)-5-pyridinyl);
Ph(3-Cl, 5-OCHF₂)—(R)CH(OH)C(O)-Aze-NH—CH₂-((5-amidino)-2-pyrimidinyl);
Ph(3-Cl, 5-OCHF₂)—(R)CH(OH)C(O)-Aze-Pab(3-F);
Ph(3-Cl, 5-OCHF₂)—(R)CH(OH)C(O)-Aze-Pab(2,6-diF);
Ph(3-Cl, 5-OCHF₂)—(R)CH(OH)C(O)-Aze-Pab(2,5-diF).
Ph(3-Cl)(5-OCHF₂)—(R)CH(OH)C(O)-Aze-Pab(OMe);
Ph(3-Cl)(5-OCHF₂)—(R)CH(OH)C(O)-Aze-Pab(OEt);
Ph(3-Cl)(5-OCHF₂)—(R)CH(OH)C(O)-Aze-Pab(OnPr);
Ph(3-Cl)(5-OCHF₂)—(R)CH(OH)C(O)-Aze-Pab(OiPr);
Ph(3-Cl)(5-OCHF₂)—(R)CH(OH)C(O)-Aze-Pab(OcBu);
Ph(3-Cl)(5-OCHF₂)—(R)CH(OH)C(O)-Aze-Pab(OH);
Ph(3-Cl)(5-OCHF₂)—(R)CH(OH)C(O)-Aze-Pab(COOcPentyl);
Ph(3-Cl)(5-OCHF₂)—(R)CH(OH)C(O)-Aze-Pab(Z);
Ph(3-Cl)(5-OCF₃)—(R)CH(OH)C(O)-Aze-Pab(OMe);
Ph(3-Cl)(5-OCF₃)—(R)CH(OH)C(O)-Aze-Pab(OCH₂-3-(5-Me-isoxazole));
Ph(3-Cl)(5-OCF₃)—(R)CH(OH)C(O)-Aze-Pab(OCH₂-3-pyridine);
Ph(3-Cl)(5-OCF₃)—(R)CH(OH)C(O)-Aze-Pab(OiBu);
Ph(3-Cl)(5-OCF₃)—(R)CH(OH)C(O)-Aze-Pab(OEt);
Ph(3-Cl)(5-OCF₃)—(R)CH(OH)C(O)-Aze-Pab(OBn);
Ph(3-Cl)(5-OCF₃)—(R)CH(OH)C(O)-Aze-Pab(OcHexyl);
Ph(3-Cl)(5-OCF₃)—(R)CH(OH)C(O)-Aze-Pab(OcBu);
Ph(3-Cl)(5-OCF₃)—(R)CH(OH)C(O)-Aze-Pab(OCH₂CH₂OPh(3-CF₃));
Ph(3-Cl)(5-OCF₃)—(R)CH(OH)C(O)-Aze-Pab(OBn(4-Cl));
Ph(3-Cl)(5-OCF₃)—(R)CH(OH)C(O)-Aze-Pab(OBn(3-MeO));
Ph(3-Cl)(5-OCF₃)—(R)CH(OH)C(O)-Aze-Pab(OBn(2-Br));
Ph(3-Cl)(5-OCF₃)—(R)CH(OH)C(O)-Aze-Pab(OBn(4-Me));
Ph(3-Cl)(5-OCF₃)—(R)CH(OH)C(O)-Aze-Pab(O-4-heptyl);
Ph(3-Cl)(5-OCF₃)—(S)CH(CH₂OH)C(O)-Aze-Pab(OMe);
Ph(3-Cl)(5-OCH₂CF₃)—(R)CH(OH)C(O)-Aze-Pab(OMe);
Ph(3-Cl)(5-OCH₂CHF₂)—(R)CH(OH)C(O)-Aze-Pab(OMe);
Ph(3-Cl)(5-OCH₂F)—(R)CH(OH)C(O)-Aze-Pab(OMe);
Ph(3-Cl)(5-OCH₂CH₂F)—(R)CH(OH)C(O)-Aze-Pab(OMe);
Ph(3-Cl)(5-OCH(CH₂F)₂)—(R)CH(OH)C(O)-Aze-Pab(OMe);
Ph(3-F)(5-OCHF₂)—(R)CH(OH)C(O)-Aze-Pab(OMe);
Ph(3-Br)(5-OCHF₂)—(R)CH(OH)C(O)-Aze-Pab(OMe);
Ph(3-Cl, 5-OCH₂CHF₂)—(R)CH(OH)C(O)-Aze-Pab(OH);
Ph(3-Cl, 5-OCH₂CH₂F)—(R)CH(OH)C(O)-Aze-Pab(OH);
Ph(3-Cl, 5-OCHF₂)—(R)CH(OH)C(O)-Pro-Pab(OMe);
Ph(3-Cl, 5-OCHF₂)—(R)CH(OH)C(O)-Aze-NH—CH₂-((2-methoxy-amidino)-5-pyridinyl);
Ph(3-Cl, 5-OCHF₂)—(R)CH(OH)C(O)-Aze-NH—CH₂-((5-methoxy-amidino)-2-pyrimidinyl);
Ph(3-Cl, 5-OCHF₂)—(R)CH(OH)C(O)-Aze-Pab(2,6-diF)(OMe); or
Ph(3-Cl, 5-OCHF₂)—(R)CH(OH)C(O)-Aze-Pab(2,5-diF)(OMe) and pharmaceutically-acceptable derivatives thereof.

7. A method of making a kit of parts as defined in any one of claims 3 to 6, which method comprises bringing a component (a), as defined in any one of claims 3 to 6, into association with a component (b), as defined in any one of claims 3 to 6, thus rendering the two components suitable for administration in conjunction with each other.

8. A kit of parts comprising:
(I) one of components (a) and (b) as defined in any one of claims 3 to 6; together with (II) instructions to use that component in conjunction with the other of the two components.

9. A method of treatment arrhythmia, which comprises administration of a combination product as defined in any one of claims 1 to 8 to a patient suffering from, or susceptible to, such a condition.

10. The use of a combination product as defined in any one of claims 1 to 8 for the manufacture of a medicament for the treatment or prophylaxis of a condition where anticoagulant therapy is indicated.

11. The use of a compound of claim 1 in WO 02/44145 or a compound of claim 20 in WO 02/44145 (or derivative thereof) or a pharmaceutically-acceptable derivative thereof and (1) a compound as defined in claim 1 of WO 01/28992 or (2) a compound of claim 34 of WO 01128992 or (3) Compound A or B or C or D (or pharmaceutically-acceptable salts thereof) for the manufacture of a medicament for the treatment or prophylaxis of a condition where anticoagulant therapy is indicated.
